(19) United States
(12) Patent Application Publication
(10) Pub. No.: US 2013/0236964 A1
(43) Pub. Date: Sep. 12, 2013

(54) METHODS, NUCLEIC ACID CONSTRUCTS AND CELLS FOR TREATING NEURODEGENERATIVE DISORDERS

(71) Applicant: RAMOT AT TEL-AVIV UNIVERSITY LTD., Tel-Aviv (IL)

(72) Inventors: Eldad MELAMED, Tel-Aviv (IL); Daniel Offen, Kfar HaRoe (IL); Yosef Levy, Modiln (IL); Pnina Green, Holon (IL)

(73) Assignee: Ramot at Tel-Aviv University Ltd., Tel-Aviv (IL)

(21) Appl. No.: 13/783,607

(22) Filed: Mar. 4, 2013

Related U.S. Application Data
(62) Division of application No. 11/130,197, filed on May 17, 2005, now abandoned.
Application No. 11/130,197 is a continuation-in-part of application No. PCT/IL03/00972, filed on Nov. 17, 2003.
(60) Provisional application No. 60/651,645, filed on Feb. 11, 2005.

(30) Foreign Application Priority Data
Nov. 17, 2002 (IL) 152905

(51) Int. Cl. C12N 5/0793

(52) U.S. Cl. CPC C12N 5/0619
USPC 435/372; 435/377

(57) ABSTRACT

A method of treating a neurodegenerative disorder is provided. The method is effected by administering to an individual in need thereof cells capable of exogenously regulatable neurotransmitter synthesis thereby treating the neurodegenerative disorder.

### RELATED APPLICATIONS

This application is a division of U.S. patent application Ser. No. 11/130,197 filed May 17, 2005, which is a continuation-in-part of PCT Patent Application No. PCT/IL03/00972 filed Nov. 17, 2003, which claims the benefit of priority of Israel Patent Application No. 152905 filed Nov. 17, 2002.

U.S. patent application Ser. No. 11/130,197 also claims the benefit of priority under 35 USC §119(e) of U.S. Provisional Patent Application No. 60/651,645 filed Feb. 11, 2005.

The contents of the above applications are all incorporated by reference as if fully set forth herein in their entirety.

### SEQUENCE LISTING STATEMENT

The ASCII file, entitled 55722SequenceListing.txt, created on Mar. 4, 2013, comprising 13,930 bytes, submitted concurrently with the filing of this application is incorporated herein by reference.

### FIELD AND BACKGROUND OF THE INVENTION

The present invention relates to neuronal-like cells capable of controllable synthesis of neurotransmitters and of cell replacement therapy using such cells for treating neurodegenerative disorders such as Parkinson's disease.

Parkinson's disease is an age-related disorder characterized by progressive loss of dopamine producing neurons in the substantia nigra of the midbrain, which in turn leads to progressive loss of motor functions manifested through symptoms such as tremor, rigidity and ataxia. Parkinson's disease can be treated by administration of pharmacological doses of the precursor of dopamine, L-DOPA (Marsden, Trends Neurosci. 9:512, 1986; Vinken et al., in Handbook of Clinical Neurology p. 185, Elsevier, Amsterdam, 1986). Although such treatment is effective in early stage Parkinson's patients, progressive loss of substantia nigra cells eventually leads to an inability of remaining cells to synthesize sufficient dopamine from the administered precursor and to diminishing pharmacogenic effect.

Studies of neurodegenerative diseases suggest that symptoms that arise in afflicted individuals are secondary to defects in local neural circuitry and cannot be treated effectively with systemic drug delivery. Consequently, alternative approaches for treating neurodegenerative diseases have emerged, such as transplantation of cells capable of replacing or supplementing the function of damaged neurons. For such cell replacement therapy to work, implanted cells must survive and integrate, both functionally and structurally, within the damaged tissue.

Parkinson's disease is the first disease of the brain for which intracerebral cell replacement therapy has been used in humans. Several attempts have been made to provide the neurotransmitter dopamine to cells of the diseased basal ganglia of Parkinson's patients by homografting adrenal medullary cells to the brain of patients (Backlund et al., J. Neurosurg. 62:169-173, 1985; Madrazo et al., New Eng. J. Med. 316:831-836, 1987). Transplantation of other donor cells such as fetal brain cells from the substantia nigra, an area of the brain rich in dopamine-containing cell bodies and also the area of the brain most affected in Parkinson's disease, has been shown to be partially effective in reversing the behavioral deficits induced by selective dopaminergic neurotoxins (Bjorklund et al., Ann. N.Y. Acad. Sci. 457:53-81, 1986; Dunnett et al., Trends Neurosci. 6:266-270, 1983).

Several cell replacement studies utilizing various non-neuronal cell types from different sources have also been conducted over the past few years. In animal models of Parkinson's disease, researchers have transplanted cells such as monocytes, bone marrow stem cells, myoblasts, fibrolasts, astrocytes and Sertoli cells (Costantini et al., 2000; Hwan-Wun et al., 1999; Linder et al., 1995; Patridge & Davies 1995; Perry & Gordon 1998; Yadid et al., 1999). In other studies, cells were transplanted after being genetically engineered with growth factor genes (e.g., glial-derived and brain-derived growth factors) to enhance survival rates, or with genes such as tyrosine hydroxylase, aromatic amino acid decarboxylase or GTP-cyclohydrolase I, which are capable of increasing dopamine synthesis in the transformed cell (Choi-Lundberg et al., 1998; Yoshimoto et al., 1995; Schwarz et al., 1999). However, these cells failed to fully acquire the structural and functional characteristics of the damaged neuronal cells and consequently proved to be therapeutically ineffective (Brundin et al., 2000).

Clinical cell replacement trials for Parkinson's patients have been conducted using fetal cells which comprise just 1-2% dopaminergic neurons (Freed et al., 1992; Freed et al., 2001; Freeman et al., 1995; Kordower et al., 1995; Kordower et al., 1998; Lindvall O., 1991; and Wenning et al., 1997). Freed et al (2001) found that fetal cell transplantation to Parkinson's patients was beneficial only to young patients (<60 years). Furthermore, several patients suffered from severe dyskinesia without levodopa treatment (“runaway dyskinesias”) due to an excessive and uncontrolled production and release of dopamine by implanted cells (Freed et al., 2001; Olanow et al., 2003). In addition, the low availability of human fetal tissue substantially limits the number of patients which could benefit from fetal cell transplantation.

The use of stem cells as a cellular source in cell replacement therapy for Parkinson's disease has been recently suggested. Indeed, replacement of damaged dopaminergic neurons with cells derived from mouse or human embryonic stem cells in experimental models of PD, demonstrated some clinical improvement (Lee, S. H., et al., 2000; Kim, J. H. et al., 2002; Ben-Hur, T., et al., 2004). However, these cells cannot be used clinically since apart from the clinical implications, they are difficult to obtain, cause immune reaction and may develop to teratomes (Hadjantonakis A K, et al., 1998).

In these respects, bone marrow derived stromal stem cells (BMSc) are of special interest since they are easily harvested, isolated, and purified and might be used for autologous transplantation (Jackson-Lewis V, Liberatore G. 2000; Azizi S. A., et al., 1998; Kopen et al., 1999).

BMSc have been established as multipotent cells with the potential to differentiate into a variety of cells such as osteoblasts, chondrocytes and adipocytes (Prockop, D. J., et al., 1997).

BMSc have also been shown to differentiate into neuron-like cells demonstrating neuronal markers (Azizi et al., 1998; Deng et al., 2001; Kopen et al., 1999; Levy et al., 2003; Sanchez-Ramos et al., 2000; Schwarz et al., 1999; Woodbury et al., 2000) and some electro-physiological functions (Kohyama et al., 2001). The cells have also been shown to express dopaminergic markers and also to secrete dopamine following depolarization (Levy et al., 2004). It was reported that bone marrow cells have the potential to migrate into injured neural tissues and to differentiate into neurons (Mahmood et al., 2001; Li et al., 2001; 2002; Kan et al., 2005). Moreover, transplantation of BMSc in mouse and rat models of Parkinson's disease resulted in beneficial effects (Li et al., 2001).

Although adult BMSc can be differentiated into neuron-like cells which are structurally compatible with implantation, engrafted BMSc may release neurotransmitters such as dopamine uncontrollably which in turn may cause severe side effects such as “runaway dyskinesia” and thus rendering the use of BMSc unsuitable for therapy of neurodegenerative disorders.

There is thus a widely recognized need for, and it would be highly advantageous to have, neuronal-like cells which are capable of controllably synthesizing neurotransmitters, such as dopamine, and thus can be utilized to effectively and safely treat neurodegenerative disorders.

### SUMMARY OF THE INVENTION

According to one aspect of the present invention there is provided a method of treating a neurodegenerative disorder which includes administering to an individual in need thereof cells capable of exogenously regulatable neurotransmitter synthesis thereby treating the neurodegenerative disorder.

According to another aspect of the present invention there is provided a method of treating a neurodegenerative disorder which includes the steps of (a) administering to an individual in need thereof cells capable of exogenously regulatable neurotransmitter synthesis; and (b) periodically exposing the individual to an agent or condition capable of regulating the synthesis of the neurotransmitter in the cells thereby treating the neurodegenerative disorder.

According to yet another aspect of the present invention there is provided a nucleic acid construct which includes a polynucleotide sequence encoding an enzyme participating in a synthesis of a neurotransmitter positioned under a control of a regulatory sequence capable of regulating expression of the enzyme in mammalian cells.

According to still another aspect of the present invention there is provided a construct system which includes a first expression construct including a first polynucleotide sequence encoding an enzyme participating in a synthesis of a neurotransmitter positioned under the transcriptional control of a first regulatory sequence and a second expression construct including a second polynucleotide sequence encoding a transactivator positioned under the transcriptional control of a second regulatory sequence, wherein the transactivator is capable of activating the first regulatory sequence to direct transcription of the first polynucleotide sequence.

According to an additional aspect of the present invention there is provided a cell comprising a nucleic acid construct which includes a polynucleotide sequence encoding an enzyme participating in a synthesis of a neurotransmitter positioned under a control of a regulatory sequence capable of regulating expression of the enzyme in the cell.

According to yet an additional aspect of the present invention there is provided a cell comprising the construct system which includes a first expression construct including a first polynucleotide sequence encoding an enzyme participating in a synthesis of a neurotransmitter positioned under the transcriptional control of a first regulatory sequence and a second expression construct including a second polynucleotide sequence encoding a transactivator positioned under the transcriptional control of a second regulatory sequence, wherein the transactivator is capable of activating the first regulatory sequence to direct transcription of the first polynucleotide sequence.

According to still an additional aspect of the present invention there is provided a method of producing cells for use in treating neurodegenerative disorders. The method includes the steps of: (i) isolating bone marrow cells; (ii) incubating the bone marrow cells in a proliferating medium capable of maintaining and/or expanding the bone marrow cells; (iii) selecting bone marrow stromal cells from the cells resulting from step (ii); and (iv) incubating the bone marrow stromal cells in a differentiating medium including at least one polyunsaturated fatty acid and at least one differentiating agent, thereby producing the cells for use in treating neurodegenerative disorders.

According to yet an additional aspect of the present invention there is provided a population of cells which includes bone marrow derived stromal cell capable of synthesizing a neurotransmitter

According to still an additional aspect of the present invention there is provided a mixed population of cells which includes bone marrow derived stromal cell capable of synthesizing at least two types neurotransmitters.

According to further features in preferred embodiments of the invention described below, the method of treating a neurodegenerative disorder, further includes exposing the individual to an agent or condition capable of regulating the synthesis of the neurotransmitter in the cells.

According to still further features in the described preferred embodiments the cells are genetically modified so as to enable the exogenously regulatable neurotransmitter synthesis.

According to still further features in the described preferred embodiments the cells are transformed with an expression construct including a polynucleotide sequence encoding an enzyme participating in the synthesis of the neurotransmitter, wherein the expression construct is designed such that expression of the polynucleotide is controllable via the agent.

According to still further features in the described preferred embodiments the agent is capable of downregulating expression of the enzyme participating in the synthesis of the neurotransmitter.

According to still further features in the described preferred embodiments the agent is capable of upregulating expression of the enzyme participating in the synthesis of the neurotransmitter.

According to still further features in the described preferred embodiments the cells are transformed with at least one expression construct including a first polynucleotide sequence encoding an enzyme participating in a synthesis of a neurotransmitter positioned under the transcriptional control of a first regulatory sequence and a second polynucleotide sequence encoding a transactivator positioned under the transcriptional control of a second regulatory sequence, wherein the transactivator is capable of activating the first regulatory sequence to direct transcription of the first polynucleotide sequence in absence of the agent.

According to still further features in the described preferred embodiments the agent is doxycyline.

According to still further features in the described preferred embodiments the transactivator is a tetracycline-controlled transactivator.

According to still further features in the described preferred embodiments the first regulatory sequence includes a tetracycline response element.

According to still further features in the described preferred embodiments the enzyme is selected from the group consisting of tyrosine hydroxylase, DOPA decarboxylase, GTP cyclohydrolase I, dopamine dopamine β-hydroxylase, glutamate decarboxylase, tryptophane-5 monooxygenase and choline acetyltransferase.

According to still further features in the described preferred embodiments second regulatory sequence includes a human neuron-specific enolase promoter.

According to still further features in the described preferred embodiments the neurodegenerative disorder is selected from the group consisting of Parkinson's disease, multiple sclerosis, amyatrophic lateral sclerosis, autoimmune encephalomyelitis, Alzheimer's disease, Stroke and Huntington's disease.

According to still further features in the described preferred embodiments the neurodegenerative disorder is Parkinson's disease.

According to still further features in the described preferred embodiments the neurotransmitter is selected from the group consisting of dopamine, norepinephrine, epinephrine, gamma aminobutyric acid, serotonin, acetylcholine, glycine, histamine, vasopressin, oxytocin, a tachykinins, cholecytokinin (CCK), neuropeptide Y (NPY), neurotensin, somatostatin, an opioid peptide, a purine and glutamic acid.

According to still further features in the described preferred embodiments the neurotransmitter is dopamine.

According to still further features in the described preferred embodiments the cells are bone marrow cells.

According to still further features in the described preferred embodiments the bone marrow cells are bone marrow stromal cells.

According to still further features in the described preferred embodiments the cells are neuron-like cells.

According to still further features in the described preferred embodiments the neuron-like cells express at least one neuronal marker.

According to still further features in the described preferred embodiments the neuronal marker is selected from the group consisting of 2′,3′-Cyclic nucleotide 3′-phosphodiesterase (CNPase), Glypican-4 (GPC4), Necdin, Nestin, Neurite growth-promoting factor 2 (NEGF-2), Neurofilament-Heavy, Neurofilament-light, Neurofilament-medium, Neuron specific enolase (NSE), Neurotrophic tyrosine kinase receptor type 2 (TRK-2), Patched homolog (PTCH), RET tyrosine kinase, Retinoic acid receptor type a (RARA), Smoothened (SMO), Vesicular monoamine transporter 2 (VMAT 2), Neuronal Nuclei (NeuN), Aryl hydrocarbon receptor/Aryl hydrocarbon receptor nuclear translocator binding element (AhR/Arnt), Ecotropic viral integration site 1 (EVI-1), Forkhead box O1A human (FKHRhu), Glycosaminoglycan (GAG), Hepatocyte nuclear factor 3P(HNF-3β), Myelin gene expression factor 2 MEF2(2), Nuclear Y box factor (NF-Y), Neural zinc fingure 3 (NZF-3), Paired box gene 3 (Pax-3), Paired box gene 6 (Pax-6), Xenobiotic response element (XRE), Aldehyde dehydrogenase 1 (Aldh1), Engrailed 1(En-1), Nurr-1, Paired-like homeodomain transcription factor 3 (PITX-3), Aromatic L-amino acid decarboxylase (AADC), Catechol-o-methyltransferase (COMT), Dopamine transporter (DAT), Dopamine receptor D2 (DRD2), GTP cyclohydrolase-1 (GCH), Monoamine oxidase B (MAO-B), Tryptophan hydroxilase (TPH) and Tyrosine hydroxilase (TH).

According to still further features in the described preferred embodiments administering of cells is effected by transplanting the cells into a brain tissue of the individual.

According to still further features in the described preferred embodiments administering of cells is effected by transplanting the cells into a healthy area of the brain of the individual.

According to still further features in the described preferred embodiments administering of cells is effected by transplanting the cells into a spinal cord of the individual.

According to still further features in the described preferred embodiments, the method of claim 1 further comprises administering to the individual at least one fatty acid.

According to still further features in the described preferred embodiments exposing of an individual is effected by oral administration of the agent to the individual.

According to still further features in the described preferred embodiments exposing of an individual is effected by infusion of the agent to the individual.

According to still further features in the described preferred embodiments the cells are genetically modified so as to enable the exogenously regulatable neurotransmitter synthesis.

According to still further features in the described preferred embodiments the cells are transformed with an expression construct including a polynucleotide sequence encoding an enzyme participating in the synthesis of the neurotransmitter, wherein the expression construct is designed such that expression of the polynucleotide is controllable via a regulatory agent.

According to still further features in the described preferred embodiments the agent is capable of downregulating expression of the enzyme participating in the synthesis of the neurotransmitter.

According to still further features in the described preferred embodiments the agent is capable of upregulating expression of the enzyme participating in the synthesis of the neurotransmitter.

According to still further features in the described preferred embodiments the cells are genetically modified to express tyrosine hydroxylase under a regulatory control of the agent, such that when the agent is absent an activator molecule binds a response element thereby upregulating expression of the tyrosine hydroxylase.

According to still further features in the described preferred embodiments the cell is a neuron-like cell devoid of endogenous activity of the enzyme participating in the synthesis of the neurotransmitter.

According to still further features in the described preferred embodiments the cell further includes a polynucleotide encoding an apoptosis inhibiting polypeptide.

According to still further features in the described preferred embodiments the proliferation medium includes DMEM, SPN, L-glutamine, FCS, 2-β-mercaptoethanol, nonessential amino acids and EGF.

According to still further features in the described preferred embodiments, step © of the method of producing cells for treating neurodegenerative disorders is effected by identifying cells expressing at least one gene selected from the group consisting of the genes listed in Table 7 above.

According to still further features in the described preferred embodiments the method of producing cells for treating neurodegenerative disorders includes prior to step (iv) incubating the cells resulting from step (iii) in an additional differentiating medium thereby predisposing the cells to differentiate into neuron-like cells.

According to still further features in the described preferred embodiments the additional differentiating medium includes at least one agent selected from the group consisting of bFGF, EGF, vitamin E, FGF8, and shh.

According to still further features in the described preferred embodiments the additional differentiating medium includes at least one polyunsaturated fatty acid.

According to still further features in the described preferred embodiments the polyunsaturated fatty acid is docosahexaenoic acid or arachidonic acid.

According to still further features in the described preferred embodiments the additional differentiation medium further includes DMEM, SPN, L-glutamine, N2 supplement and FCS.

According to still further features in the described preferred embodiments the at least one polyunsaturated fatty acid in the differentiating medium is docosahexaenoic acid or arachidonic acid.

According to still further features in the described preferred embodiments the at least one differentiating agent is selected from the group consisting of BHA ascorbic acid, BDNF, GDNF, NT-3, IL-1β, NTN, TGFβ3 and dbcAMP.

According to still further features in the described preferred embodiments the differentiating medium further includes DMEM, SPN, L-glutamine, N2 supplement and retinoic acid.

According to still further features in the described preferred embodiments the method of producing cells for treating neurodegenerative disorders is further effected by prior to step (iv) transforming the cells resulting from step (iii) with the nucleic acid construct of the present invention.

According to still further features in the described preferred embodiments, step (i) is affected by aspiration.

According to still further features in the described preferred embodiments, step (iii) is affected by harvesting surface adhering cells and/or by flow cytometry.

According to still further features in the described preferred embodiments the neurotransmitter is dopamine.

According to still further features in the described preferred embodiments the at least two types of neurotransmitters include dopamine.

According to still further features in the described preferred embodiments the least two types of neurotransmitters include serotonin.

The present invention successfully addresses the shortcomings of the presently known methods of treating neurodegenerative diseases by providing neuronal-like cells capable of controllable synthesis of neurotransmitters and of cell replacement therapy using such cells for treating neurodegenerative disorders such as Parkinson's disease.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The patent or application file contains at least one drawing executed in color. Copies of this patent or patent application publication with color drawings will be provided by the Office upon request and payment of the necessary fee.

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIGS. 1A-1D illustrate light microscope images of non-differentiated human bone-marrow stromal cells (hBMSc). The hBMSc were cultured in a “proliferation medium” (described in Example 1 of the Examples section below) and were grown to 80-90% confluency over a time period of approximately 15 days. The plastic-adherent hBMSc had a round or spindle body shape (FIG. 1A) or a flat body shape (FIGS. 1B-D).

FIGS. 2A-2F illustrates flow cytometer analyses of non-differentiated hBMSc. Fifteen-day-old hBMS cells were stained for the presence of surface markers CD20 (FIG. 2A), CD5 (FIG. 2B), CD45 (FIG. 2C), CD11b (FIG. 2D) and CD34 (FIG. 2E) (characteristic of lymphohematopoietic cells) and with CD90 (Thy-1; a protein which is expressed during synaptogenesis) (FIG. 2F). The cells were analyzed with FACSCalibur™ flow cytometer (Becton Dickinson) equipped with an argon ion laser (adjusted to an excitation wavelength of 488 nm) and the CELLQuest™ software program (Becton Dickinson). The analyses show that the hBMS cells did not express any of the lymphocyte-associated markers but positively expressed the Thy-1 protein.

FIGS. 3A-3F illustrate light microscope images of adult hBMSc differentiated into neurons after increasing time intervals. Cultured hBMSc were transferred from the “proliferating medium” into the “additional differentiating medium” (see Examples 1-2 of the Examples section below). Following 24 hr incubation in the “additional differentiating medium”, the hBMSc were transferred into the “differentiating medium” (see Example 2 of the Examples below). The plastic-adherent cells were transformed into neuronal-like cells having a spindle shaped cell body and long branching processes that appeared as early as three hours post differentiation induction (FIG. 3B) and continued to appear up to 72 h following differentiation induction (FIGS. 3C-3F). FIG. 3A is a light microscope image of undifferentiated hBMSc.

FIG. 4 illustrates ³H-thymidine incorporation (indicative of cell proliferation) in hBMSc that have been cultured in the “differentiating medium” and in the “additional differentiating medium” (see Example 2 of the Examples below). The ³H-thymidine incorporation into differentiating cells was reduced by about 45% and 90%, as compared with the non-differentiating cells, following 16 and 39 hr incubation periods, respectively.

FIGS. 5A-G illustrate reverse transcriptase RT-PCR, real-time PCR, and northern blot analyses of RNA extracted from neuronal markers in non-differentiated and in differentiating hBMSc. FIGS. 5A and 5B illustrate RT-PCR analysis designed for identifying neuronal transcripts. FIGS. 5C-5D illustrates Northern blot analysis and real-time PCR analyses (FIG. 5E), which utilized a ³²P-labled PCR product of NEGF2 as a probe. FIG. 5F illustrates Northern blot analysis, which utilized ³²P-labled PCR product of neurofilament 200 (NF-200) as a probe. FIG. 5G illustrates northern blot analysis, which utilized a ³²P-labled PCR product of neuron specific enolase (NSE) as a probe.

FIGS. 6A-H illustrate fluorescent microscope images of neural markers in hBMSc cultured in “differentiation medium” (see Example 2 of the Examples below) for 12 hr to 5 days. Antibody-labeled neuron nuclei-specific marker (NeuN; expressed after 12 hr). (FIG. 6A); neurofilament heavy (NF-200; expressed after 24 hr) (FIG. 6B), neuron specific enolase (NSE; expressed after 48 hr), (FIG. 6C) nestin (expressed after 48 hr) (FIG. 6D) and double staining of α-Synuclein (FIG. 6E) and β-tubulin III (FIG. 6F) (expressed after 48 hr) are illustrated. FIG. 6G illustrates antibody-labeled glial fibrillary acidic protein (GFAP; expressed after 48 hr) and FIG. 6H illustrates antibody-labeled β-tubulin III (expressed after 5 days).

FIGS. 7A-F illustrate Western blot analyses of differentiated hBMSc indicating elevated expressions of neuron specific enolase (NSE; FIGS. 7A-B) neuron nuclei-specific marker (NeuN; FIG. 7C-D) and nestin (FIG. 7E-F).

FIGS. 8A-B illustrates light microscope images of neuron-like differentiated hBMSc prior to (FIG. 8A) and following (FIG. 8B) 28 days of incubation in “long-term differentiation medium” (see Example 5 of the Examples below).

FIGS. 9A-R illustrate light and fluorescent microscope images of hBMSc which were incubated in the “proliferation medium” (see Example 1 of the Examples below) or in the “long-term differentiation medium” (see Example 5 of the Examples below). FIGS. 9A-F illustrates antibody-labeled expression of the neuronal marker β-tubulin III in the differentiated cells. FIG. 9G-L illustrates antibody-labeled expression of the neuronal marker MAP-2 in the differentiated cells. FIG. 9M-R illustrates antibody-labeled expression of the neuronal marker nestin in the differentiated cells.

FIG. 10 illustrates RT-PCR analyses designed for identifying dopaminogenic markers in hBMSc, transcription factors of dopaminergic neurons (Aldh1, Nurr1, Prd1, En1) sonic hedgehog receptors (SMO, PTCH) and proteins of the dopaminergic system (GTPCH1, AADC, MAO, COMT, DAT, D2DR), which have been cultured for 12-72 hr in the “differentiation medium” (see Example 2 of the Examples below).

FIGS. 11A-H illustrate expression of tyrosine hydroxylase (TH) in differentiated hBMSc. FIG. 11A illustrates real-time PCR analysis indicating an elevated TH transcription in the differentiated cells. FIGS. 11B-C illustrates Western blot analysis indicating an elevated level of TH in the differentiated cells. FIGS. 11C-H illustrates fluorescent microscope images highlighting antibody-labeled TH expression in the differentiated cells. FIG. 11D is a control; FIG. 11E following six hours; FIG. 11F following twelve hours; FIG. 11G following 34 hours; FIG. 11H following 48 hours.

FIGS. 12A-C illustrate confocal fluorescent microscope images of hBMSc which have been incubated for five days in the “differentiation medium” (see Example 2 of the Examples below) highlighting antibody-labeled vesicular monoamine transporter 2 (VMAT-2) in the differentiated cells. FIG. 12A is a confocal fluorescent microscope image prior to incubation. FIG. 12B is a confocal fluorescent microscope image following incubation. FIG. 12C is an enlarged photograph of FIG. 12 B.

FIGS. 13A-H illustrate flow cytometer analyses of hBMSc which have been differentiated for 48 hr in the “differentiation medium” (see Example 2 of the Examples below). The cells were stained for the presence of D2 dopamine receptor (FIGS. 13A-B), NSE (FIGS. 13D and 13G), NF-200 (FIGS. 13C and 13F), and TH (FIGS. 13E and 13H), and were analyzed with FACSCalibur™ flow cytometer (Becton Dickinson). The analysis shows that a D2 dopamine receptor, NSE, NF-200 and TH were present in the differentiated hBMSc.

FIGS. 14A-D illustrate HPLC analyses of differentiating hBMSc. FIG. 14A illustrates the levels of dopamine measured in the supernatant of hBMSc which have been incubated for 0-96 hr in the “differentiation medium” (see Example 2 of the Examples below). FIG. 14B illustrates the levels of dopamine measured in the supernatant of hBMSc which have been incubated for 0-96 hr in the “differentiation medium” followed by an additional incubation of 10 minutes in 56 mM KCl solution. FIG. 14C illustrates the levels of the dopamine precursor DOPA measured in the supernatant of hBMSc which have been incubated in the “differentiation medium” for 0-72 hr. FIG. 14D illustrates the levels of dopamine metabolite DOPAC measured in the supernatant of hBMSc which have been cultured in the “differentiation medium” for 0-50 hr.

FIGS. 15A-F illustrate the effect of mouse bone-marrow stromal cells (mBMSc) transplantation on the recovery of amphetamine-induced motor rotation in a rat model for Parkinson's disease. mBMSc were isolated from green fluorescent-protein marked transgenic mice (GFP-Tg). The isolated mBMSc were induced for neural differentiation and transplanted into the nigra of 6-OHDA lesioned rats. The rats were then treated with amphetamine and were examined for rotational response over a period of 45 days post transplantation. FIG. 15A illustrates the changes in rotation rates over time followed transplantation indicating diminishing rotations (indicative of improvement of motor function) 45 days post transplantation. FIG. 15B illustrates the changes in relative rotations (as compared with non-treated mice) over time followed transplantation indicating 97.9% decrease in relative rotations 45 days post transplantation. FIGS. 15C-D illustrates fluorescent microscope images highlighting transplanted mBMSc present in the substania nigra of treated mice 45 days post transplantation. FIG. 15E-F illustrates fluorescent microscope images highlighting transplanted mBMSc present in the striatum of treated mice 45 days post nigral transplantation.

FIGS. 16A-D illustrate dopaminergic and serotoninergic activities in differentiated human bone marrow stromal cells (hBMSc). FIGS. 16A-B illustrate western blot analysis indicating expression of tryptophan hydroxylase. FIG. 16C illustrates RT-PCR analysis indicating tryptophan hydroxylase transcription. FIG. 16D illustrates HPLC analysis indicating synthesis of DOPAC (a dopamine metabolite) and 5HIAA (a serotonin metabolite).

FIG. 17 illustrates a construct of an expression vector containing Nurr-1 encoding sequence inserted within pcDNA-3.1A (Invitrogene).

FIG. 18 illustrates a construct designed for a negative selection of dopaminergic cells. The construct includes the human TH (tyrosine hydroxylase) promoter inserted in pMOD (InvivoGene) upstream of the “suicide gene” HSV1-tk (herpes simplex virus type 1 thymidine kinase encoding toxic gancyclovir).

FIGS. 19A-B illustrate the Tet-off Tet-on system for doxycyline-controlled expression of tyrosine hydroxylase (TH). FIG. 19A illustrates the regulator and response constructs of the system. FIG. 19B illustrates a schematic diagram describing the system mode of action.

FIGS. 20A-D illustrate fluorescent microscope images of differentiated BMSc of a GFP-Tg mouse (mBMSc). FIGS. 20A and 20B illustrates fluorescent microscope image of morphological changes induced by “differentiation medium” (see Example 1). FIGS. 20C and 20D illustrates a fluorescent microscope image highlighting antibody-labeled A2B5 (a marker of oligodendrocyte precursor) expressed in the NT-3 induced mBMSc.

FIG. 21 illustrates changes in the rotational performance of mice expressing SOD1 (an animal model of amyotrophic lateral sclerosis; ALS) over time. The SOD1 mice suffered a substantial reduction in rotational performance as compared with the wild type mice and became completely paralyzed at the age of 4-5 months.

FIG. 22 illustrates a PCR analysis of different tissues of a female mouse sampled one week after male-derived mBMSc had been transplanted into the cisterna magna. The analysis detects Chromosome Y (indicative of the transplanted cells) in the spinal cord of the female mouse but not in other tissues.

FIG. 23 illustrates rotarod performance (indicative of rotational behavior) of wild-type mice which received mBMSc transplantation into their spinal cords at the age of 7 weeks. Mice which were treated with saline injection were use as control. The transplantation of mBMSc did not affect the rotational behavior of the wild type mice.

FIG. 24 illustrates a rotarod performance (indicative of rotational behavior) of SOD1 mice (model of amyotrophic lateral sclerosis) which received mBMSc transplantation into their spinal cords at the age of 7 weeks. Mice which were treated with saline injection were use as control. The transplantation of mBMSc significantly improved the rotational behavior of the SOD1 mice, as compared with the saline-treated control.

FIGS. 25A-B are fluorescent microscope images showing the loss of dopaminergic cell bodies in the substantia nigra (SN) following intrastriatal injection of 6-OHDA using an anti-tyrosine hydroxylase (TH) antibody. FIG. 25A illustrates the SN cell bodies of a treated hemisphere. FIG. 25B illustrates the SN cell bodies of a non-treated hemisphere.

FIG. 26 is a graph illustrating the reduction in amphetamine-induced rotational behavior following intrastriatal transplantation of differentiated bone marrow stem cells in 6-OHDA lesioned mice.

FIGS. 27A-G are fluorescent microscope images of EGFP-positive cells in various brain areas along the nigro-striatum dopaminergic track using a goat anti-GFP antibody. FIG. 27A is a diagram of the brain illustrating various brain areas. FIGS. 27B-G are fluorescent microscope images of EGFP-positive cells; FIG. 27B illustrates EGFP-positive cells in the striatum; FIG. 27C illustrates EGFP-positive cells in the lateral ventricle. FIG. 27D illustrates EGFP-positive cells in the internal capsule. FIG. 27E illustrates EGFP-positive cells in the medial forebrain bundle. FIG. 27F illustrates EGFP-positive cells in the pyramidal cell layer. FIG. 27G illustrates EGFP-positive cells in the medial globus pallidus.

FIGS. 28A-I are fluorescent microscope images of tyrosine hydroxylase (TH) positive cells using an anti-TH antibody and EGFP positive cells using an anti-EGFP antibody in a subpopulation of transplanted differentiated EGFP bone marrow derived stem cells in striatum. FIGS. 28A, 28D and 28G are fluorescent microscope images illustrating EGFP positive cells; FIGS. 28B, 28E and 28H are fluorescent microscope images illustrating TH positive cells; FIGS. 28C, 28F and 28I are merged fluorescent microscope images illustrating both EGFP and TH positive cells.

FIGS. 29A-C are fluorescent microscope images depicting tyrosine hydroxylase (TH) positive cells using an anti-TH antibody and EGFP positive cells using an anti-EGFP antibody in a subpopulation of transplanted differentiated EGFP bone marrow derived stem cells which have migrated to the subtantia nigra. FIG. 29A is a fluorescent microscope image illustrating EGFP positive cells. FIG. 29B is a fluorescent microscope image illustrating TH positive cells. FIG. 29C is a merged fluorescent microscope image illustrating both EGFP and TH positive cells.

FIGS. 30A-D are photographs of sections of 6-OHDA lesioned and unlesioned rat brain following mouse BMSC transplantation demonstrating the preferential survival of BMSC transplanted cells in the lesioned areas. FIG. 30A is a photograph illustrating TH positive cells in the 6-OHDA lesioned rat striatum. FIG. 30B is a photograph illustrating TH positive cells in the unlesioned rat striatum. FIG. 30C is a photograph illustrating mouse M6 positive cells in the 6-OHDA lesioned rat striatum. FIG. 30D is a photograph illustrating mouse M6 positive cells in the unlesioned rat striatum.

FIG. 31 is a bar graph illustrating the average number of M6 positive cells per slide in the lesioned and unlesioned rat brain hemisphere.

FIGS. 32A-I illustrate the migration of non-differentiated BMSCs across the mouse brain towards the site of 6-OHDA lesion. FIGS. 32A, 32D and 32G are schematic representations of the brain indicating the area of the brain which was sectioned and analyzed. FIGS. 32B, 32E and 32H are fluorescent microscope images of GFP expressing non-differentiated BMSCs sectioned from the BMSC injected striatum, the corpus callosum and the 6-OHDA injected striatum respectively. FIGS. 32C, 32F and 32I are photographs of iron transfected non-differentiated BMSCs sectioned from the BMSC injected striatum, the corpus callosum and the 6-OHDA injected striatum respectively.

FIGS. 33A-G illustrate the migration of differentiated BMSc across the mouse brain towards the site of 6-OHDA lesion. FIGS. 33A, 33B and 32E are schematic representations of the brain indicating the area of the brain which was sectioned and analyzed. FIGS. 33C and 33F are fluorescent microscope images of GFP expressing differentiated BMSc sectioned from the corpus callosum and the 6-OHDA injected striatum respectively. FIGS. 33D and 33G are photographs of iron transfected non-differentiated BMSc sectioned from the corpus callosum and the 6-OHDA injected striatum respectively.

FIGS. 34A-K are graphs illustrating the results obtained from flow cytometric analysis showing that non-differentiated human BMSc exhibit mesenchymal characteristics. FIGS. 34A-F are plot graphs illustrating flow cytometric analysis of non-differentiated human BMS cells incubated with anti CD29 antibody, anti CD44 antibody, anti CD105 antibody, anti CD45 antibody, anti CD19 antibody and anti CD34 antibody respectively at incubation day 0 and incubation day 14 in proliferation medium. FIGS. 34G-J are scatter graphs illustrating flow cytometric analysis following double staining of non-differentiated BMSc. FIG. 34G illustrates flow cytometric analysis of human BMSc at incubation day 0 with both anti CD29 and anti CD40 antibodies. FIG. 34H illustrates flow cytometric analysis of human BMSc at incubation day 23 with both anti CD29 and anti CD40 antibodies. FIG. 34I illustrates flow cytometric analysis of human BMSc at incubation day 0 with both anti CD29 and anti CD105 antibodies. FIG. 34J illustrates flow cytometric analysis of human BMSc at incubation day 23 with both anti CD29 and anti CD105 antibodies. FIG. 34 K is a bar graph illustrating the percent of cells which stain positive for cell surface mesenchymal markers (CD29, CD105, CD44 and CD90) and cell surface hematopoietic markers (CD45, CD34, CD19, CD20, CD5, CD11B, FMC7) at day 0 and day 14 of incubation in proliferation medium.

FIG. 35 is a photograph illustrating RT-PCR analysis of neuronal specific transcripts (RARA, NFEM, TRK-2, and GPC4) transcription factors of dopaminergic neurons (Aldh1, Nurr1, Prd1, En1) sonic hedgehog receptors (SMO, PTCH), proteins of the dopaminergic system (AADC, COMT, GCH1) on non differentiated human BMSc. GAPDH was used as a positive control.

FIG. 36 is a bar graph illustrating the presence of neuronal and dopaminergic specific transcription factors in non-differentiated human BMSc as measured by DNA Protein array analysis.

FIGS. 37A-D are line graphs illustrating the increase in neuronal specific markers (NSE, NF-H, and NEGF2 respectively) following incubation of human BMSc in differentiation medium as measured by Northern Blot Analysis. FIG. 37D is a line graph illustrating the increase in nestin following incubation of human BMSc in differentiation medium as measured by flow cytometric analysis.

FIGS. 38A-D are line graphs illustrating the increase in neuronal specific markers (Neu N, NSE and nestin) and the dopamine specific marker TH following incubation of human BMSc in differentiation medium as measured by Western Blot Analysis.

FIG. 39 is a bar graph illustrating the effect DHA and AA treatment has on the increase of axon length as indicated by MAP-2 staining. The neurite length was determined with the ImagePro software and 20 neurons were measured in every case. The total neurite length of neuron-like cells was determined by measuring the individual neurite-like extensions lengths with the ImagePro Software and summing them per neuron.

FIGS. 40A and 40B are fluorescent microscope images showing an increase in axon length prior to (FIG. 40a) and following (FIG. 40b) DHA and AA treatment. MAP-2 staining is seen in pink and dapi nuclear dye is seen in red.

FIG. 41 is a bar graph illustrating the enhanced synaptophysin expression following differentiation in the presence of DHA and AA.

### DESCRIPTION OF THE PREFERRED EMBODIMENTS

The present invention is of genetically modified cells capable of controllable synthesis of neurotransmitters and of methods of generating and using such cells in cell replacement therapy of neurodegenerative disorders.

The principles and operation of the present invention may be better understood with reference to the drawings and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

Neurodegenerative disorders which are characterized by loss of neuronal functions, such as Parkinson's disease, cannot be efficiently treated using conventional drug therapy since such drugs have no effect on the underlying disease process which is typically caused by neuronal degeneration. Consequently, drug therapy can not fully compensate for the increasing loss of neuronal cells.

Although prior art cell replacement approaches have been successful when tested in animal models (U.S. Pat. No. 6,528,245; Schwartz et. al., 1999; Li et al., 2001; Costantini et al., 2000; Hwan-Wun et al., 1999; Linder et al., 1995; Patridge & Davies 1995; Perry & Gordon 1998; Yadid et al., 1999; Choi-Lundberg et al., 1998; and Yoshimoto et al., 1995), these approaches suffers from several inherent limitations which may prevent their use in human patients.

While conceiving the present invention, the present inventors realized that in order to provide safe and effective cell-replacement therapy of neurodegenerative diseases, such as Parkinson's disease, one requires cells which can be easily harvested and manipulated, are devoid of immune problems and above all be capable of synthesizing neurotransmitters, such as dopamine, in response to an external stimulus.

Although neuron-like bone marrow stromal cells (BMSc) capable of synthesizing neurotransmitters have been described by prior art studies (see, for example, U.S. Pat. No. 6,528,245 and Sanchez-Ramos et al. (2000), Woodburry et al. (2000), Woodburry et al. (J. Nerosci. Res. 96:908-917, 2002), and Deng et al. (Biophys. Res. Commun. 282:148-152, 2001), these studies did not demonstrate neurotransmitter production by such differentiated BMSc.

Notwithstanding the above, even if such prior art cells produced neurotransmitters, use thereof in cell replacement therapy would not be sensible since constitutive neurotransmitter synthesis from implanted cells can lead to formation of severe side effects such as runaway dyskinesia.

Thus, according to one aspect of the present invention there is provided a method of treating a neurodegenerative disorder. As used herein, the phrase “neurodegenerative disorder” refers to any disorder, disease or condition of the nervous system (preferably CNS) which is characterized by gradual and progressive loss of neural tissue, neurotransmitter, or neural functions. Examples of neurodegenerative disorder include, Parkinson's disease, multiple sclerosis, amyatrophic lateral sclerosis, autoimmune encephalomyelitis, Alzheimer's disease, Stroke and Huntington's disease.

The term “treating” as used herein refers to refers to reversing, alleviating, inhibiting the progress of, or preventing the disorder, disease or condition to which such term applies, or one or more symptoms of such disorder or condition. The term “treatment” or “therapy” as used herein refer to the act of treating.

The method is affected by administering to an individual in need thereof cells capable of exogenously regulatable neurotransmitter synthesis thereby treating the neurodegenerative disorder.

Cells capable of exogenously regulatable neurotransmitter synthesis (i.e., cells which produce neurotransmitters on demand), such as the cells described in greater detail herein below, are better suited for cell replacement therapy since the undesired side effects associated with cells utilized by prior art approaches described above can be eliminated.

A neurotransmitter according to the teaching of the present invention can be any substances which is released on excitation from the axon terminal of a presynaptic neuron of the central or peripheral nervous system and travel across the synaptic cleft to either excite or inhibit the target cell. The neurotransmitter can be, for example, dopamine, norepinephrine, epinephrine, gamma aminobutyric acid, serotonin, acetylcholine, glycine, histamine, vasopressin, oxytocin, a tachykinin, cholecytokinin (CCK), neuropeptide Y (NPY), neurotensin, somatostatin, an opioid peptide, a purine or glutamic acid. Preferably the neurotransmitter is dopamine.

Cellular synthesis of such neurotransmitters is directed by a pathway of enzymes which cooperate in converting precursor molecules into the active neurotransmitter. For example, dopamine is synthesized in dopaminergic neurons from L-dihydroxyphenylalanine (L-DOPA) through the action of DOPA decarboxylase, while L-DOPA is produced from tyrosine through the action of tyrosine hydroxylase. Norepinephrine is produced in noreinephrinergic neurons from dopamine through the action of dopamine β-hydroxylase. Epinephrine is produced in epinephrinergic neurons from norepinephrine through the action of phenylethanolamine N-methyltransferase. Gama aminobutiric acid (GABA) is produced in GABAergic neurons from glutamate through the action of glutamate decarboxylase. Serotonin is produced in serotoninergic neurons from tryptophane through a two-step process by tryptophane-5-monooxygenase (hydroxylation) and by aromatic L-amino acid decarboxylase (decarboxylation). Acetylcholine is produced in cholinergic neurons from choline and acetyl-CoA through the action of choline acetyltransferase (http://wwwdotindstatedotedu/theme/mwking/nervesdothtml).

The cells utilized by the present invention can be any actively growing cells, preferably bone marrow derived cells, more preferably bone marrow stromal cells (BMSc). The BMSc can be isolated from the iliac crest of an individual by aspiration followed by culturing in a proliferation medium capable of maintaining and/or expanding the isolated cells ex vivo. Prior to isolation of the BMSc from a subject, the subject may be administered with fatty acids as described herein below. The proliferation medium may be DMEM, alpha-MEM or DMEM/F12. Preferably, the proliferation medium is DMEM. Preferably, the proliferation medium further comprises SPN, L-glutamine, a serum (such as fetal calf serum or horse serum), 2-β-mercaptoethanol, nonessential amino acids and EGF such as described in Example 1 of the Examples section which follows.

The proliferating cells may be directly differentiated to neuron-like cells, or transformed using the constructs and transformation methods described herein below prior to their differentiation to neuron-like cells.

As used herein “neuron-like cells” are cells which display neuronal activity (e.g., neurotransmitter synthesis). Such cells typically display neuronal cell morphology and express at least one neuronal marker such as detailed in Table 7 of the Examples section which follows.

Differentiation to neuron-like cells can be effected by incubating the cells in differentiating media such as those described in U.S. Pat. No. 6,528,245 and by Sanchez-Ramos et al. (2000); Woodburry et al. (2000); Woodburry et al. (J. Neurisci. Res. 96:908-917, 2001); Black and Woodbury (Blood Cells Mol. Dis. 27:632-635, 2001); Deng et al. (2001), Kohyama et al. (2001), Reyes and Verfatile (Ann. N.Y. Acad. Sci. 938:231-235, 2001) and Jiang et al. (Nature 418:47-49, 2002).

BMSc are preferably incubated in an “additional differentiation medium” for at least 24 hours, preferably 48 hours, prior to their incubation in a “differentiation medium”.

A suitable differentiation medium may be any growth medium capable of predisposing the cells to neuron-like differentiation, such as a growth medium supplemented with the mitogen basic fibroblast growth factor (bFGF). The differentiating media (including the additional differentiating medium) may be DMEM or DMEM/F12, preferably DMEM. Preferably, the differentiating media further comprises SPN, L-glutamine, a supplement (such as N2 or B27), retinoic acid and a serum (such as fetal calf serum, fetal bovine serum or horse serum) such as the differentiating media described in Example 2 of the Examples section which follows. The “additional differentiating medium” may also include other agents such as growth factors and vitamins e.g., bFGF, EGF, vitamin E, FGF8, and shh.

Preferably, the “differentiating medium” includes at least one neuronal differentiating agent such as BHA, ascorbic acid, BDNF, GDNF, NT-3, IL-1β, NTN, TGFβ3 and dbcAMP.

Preferably, differentiation is effected in the presence of at least one type of long-chain polyunsaturated fatty acids (PUFA). Long-chain polyunsaturated fatty acids, such as docosahexaenoic acid (DHA) and arachidonic acid (AA), are known to be essential for proper neuronal development and function. DHA has been shown to modulate the biosynthesis of phosphatidyl serine (PS) one of the major anionic phospholipids in neuronal membranes [Green and Yavin, J. Neurochem. 65: 2555-2560, 1995; Garcia et al., J. Neurochem. 70:24-30, 1988]. In neuronal cell culture studies it has been demonstrated that DHA has antiapoptotic effects, probably related to DHA-induced PS accumulation (Kim et al., J. Biol. Chem. 275: 35215-35223, 2000; Kim et al., J. Mol. Neurosci. 16: 223-227, 2002; and Akbar and Kim, J. Neurochem. 82: 655-665, 2002).

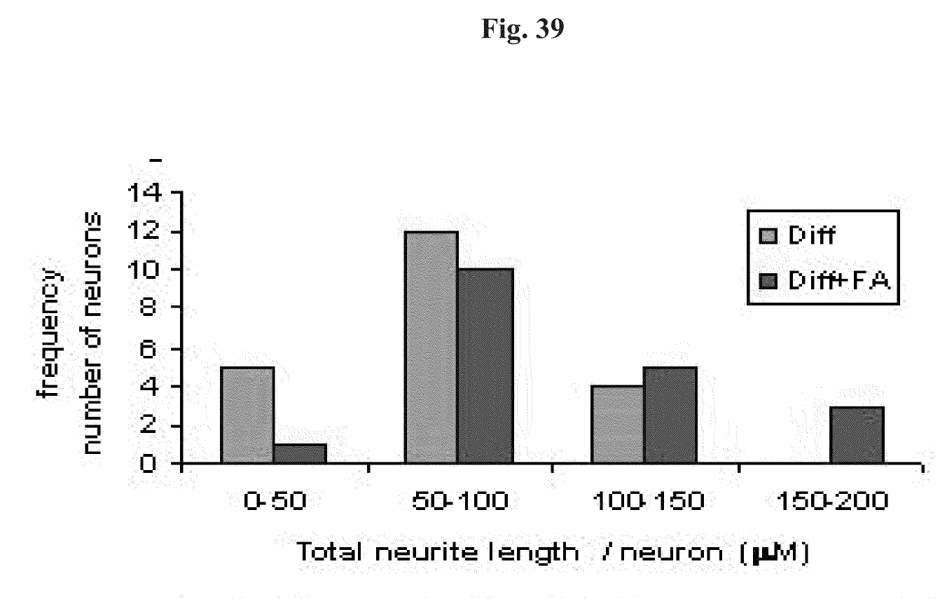

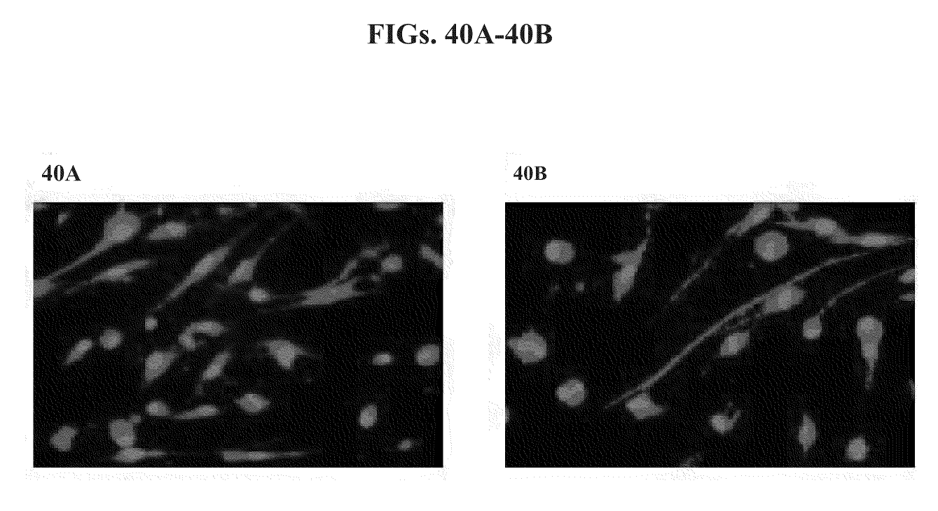

As illustrated in Example 21 of the Examples section, addition of 40 μM each of DHA (Sigma) and arachidonic acid (AA) to cultured BMSc substantially increased the expression of synaptophysin, an effect which may mediate maturation of neurotransmitter secreting vesicles in the synapses of neuron-like BMSc. The addition of these PUFA also caused an increase in neurite growth (as shown in FIG. 39 and FIGS. 40A and 40B of Example 21). Of note, the addition of DHA and AA as described above resulted in an increase in cellular PUFA and a fatty acid profile approaching that of normal neural tissue, in contrast to cells grown without the addition of PUFA, which displayed a fatty acid profile of non-neural type cells (See Example 21 of the Examples section herein below).

Thus, according to a preferred embodiment of the present invention, differentiation of BMSc to neuron-like cells is effected by incubating the cells in a differentiating medium (either a differentiating medium or an additional differentiating medium) (as described herein above) which includes at least one polyunsaturated fatty acid, such as DHA. The concentration of DHA in the predifferentiation medium is between 1-100 μM, preferably between 20-50 μM. Preferably, the differentiating medium also includes a second polyunsaturated fatty acid, such as arachidonic acid.

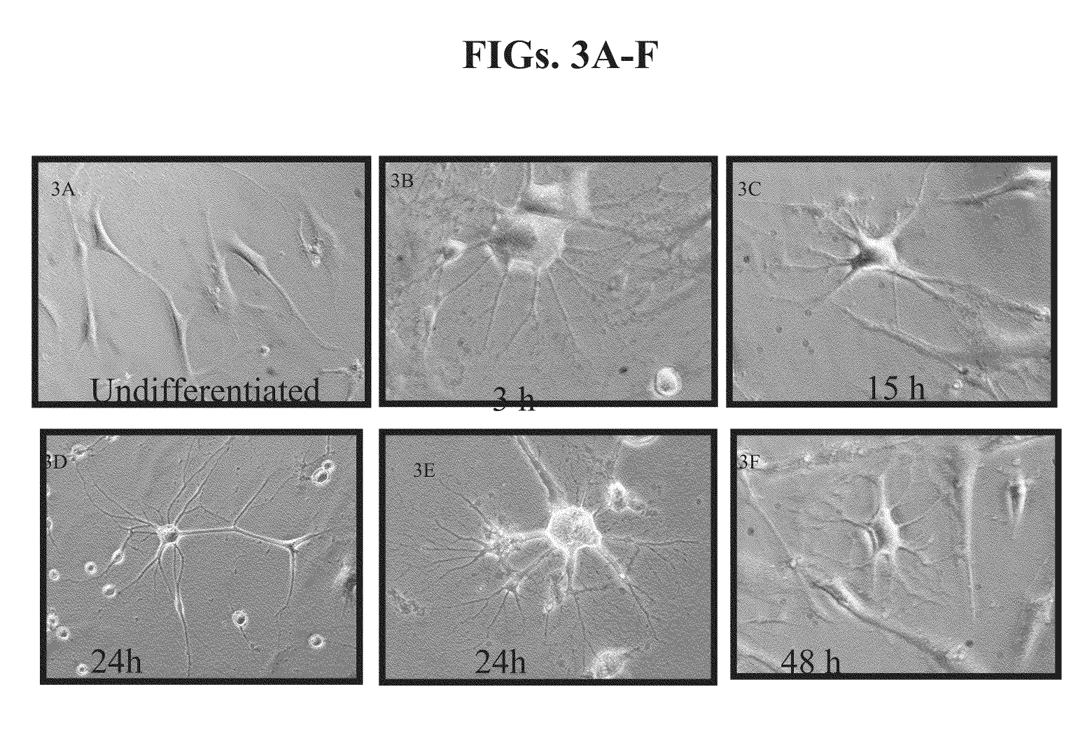

The neuronal-like cells resulted from the procedure described hereinabove typically exhibit neuronal cell morphology (illustrated in FIGS. 3A-3F) and express at least one neuronal marker such as, for example, a neural protein such as Glypican-4 (GPC4), Necdin, Nestin, Neurite growth-promoting factor 2 (NEGF-2), Neurofilament-heavy, Neurofilament-light, Neurofilament-medium, Neuron specific enolase (NSE), Neurotrophic tyrosine kinase receptor type 2 (TRK-2), Neuronal Nuclei (NeuN), RET tyrosine kinase or Retinoic acid receptor type a (RARA), and oligo-dendrocytes protein such as 2′,3′-Cyclic nucleotide 3′-phosphodiesterase (CNPase). Alternatively, the neuronal marker may be a neuronally active transcription factor such as, for example Aryl hydrocarbon receptor/Aryl hydrocarbon receptor nuclear translocator binding element (AhR/Arnt), Ecotropic viral integration site 1 (EVI-1), Forkhead box O1A human (FKHRhu), Glycosaminoglycan (GAG), Hepatocyte nuclear factor 3β (HNF-3β), Myelin gene expression factor 2 MEF2(2), Nuclear Y box factor (NF-Y), Neural zinc finger 3 (NZF-3), Paired box gene 3 (Pax-3), Paired box gene 6 (Pax-6) or Xenobiotic response element (XRE). Preferably if the cells are required to treat Parkinson's disease, then they should also express a dopaminergic marker such as a dopaminergic transcription factor such as Aldehyde dehydrogenase 1 (Aldh1), Engrailed 1(En-1), Nurr-1 or Paired-like homeodomain transcription factor 3 (PITX-3) or a dopaminergic protein such as Aromatic L-amino acid decarboxylase (AADC), Catechol-o-methyltransferase (COMT), Dopamine transporter (DAT), Dopamine receptor D2 (DRD2), GTP cyclohydrolase-1 (GCH), Monoamine oxidase B (MAO-B), Tryptophan hydroxylase (TPH), Vesicular monoamine transporter 2 (VMAT 2), Patched homolog (PTCH), Smoothened (SMO) or Tyrosine hydroxilase (TH).

Expression of neural markers is confirmed using methods, such as immunoassays, flow cytometery, RT-PCR, Northern blot analysis, Western blot analysis, Real-time PCR and HPLC methods such as described in Examples 3-9, 12 and 20 in the Examples section that follows.

While reducing the present invention to practice, the inventors uncovered that undifferentiated BMSc also express neuronal markers and dopaminergic markers as described in Example 20 and therefore may possess a neural predisposition. Thus both neuronally differentiated and non-differentiated BMSc may be used in accordance with this aspect of the present invention. Preferably, however, neuronally differentiated cells are used for this aspect of the present invention since they show enhanced expression for both neuronal and dopaminergic markers as described in Example 20 of the Examples section herein below.

It will be appreciated that replacement therapy of Parkinson's disease utilizing dopaminergic-only cells may in the long run lead to imbalances in non-dopaminergic transmitter systems and subsequently to side effects such as wearing-off and dyskinesia (Nicholson & Brotchie, Eur J Neurol. 3:1-6, 2002). Accordingly, agents which target non-dopaminergic systems and which are capable of preventing, or limiting, the expression of involuntary movements in Parkinson's disease, have been suggested for use in treating Parkinson's patients (Djaldetti & Melamed, J Neurol. 2:30-5, 2002; Muller, T., Expert Opin. Pharmacother. 2:557-72, 2001; Jenner, P. J. Neurol. 2:43-50, 2000). Furthermore, serotonin receptors have been identified as potential therapeutic targets in Parkinson's disease. (Nicholson & Brotchie, Eur J Neurol. 3:1-6, 2002).

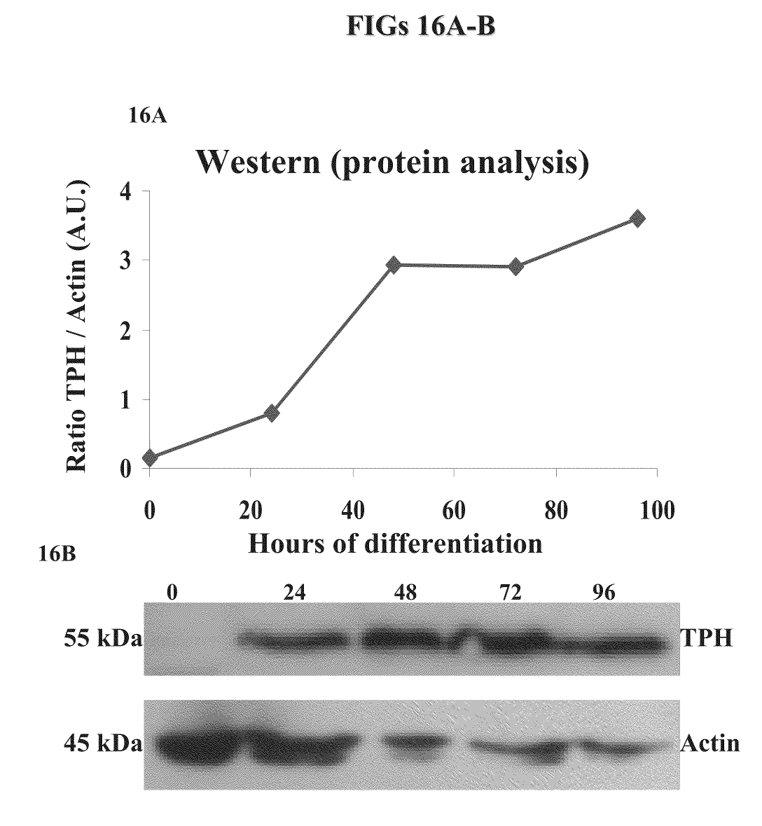

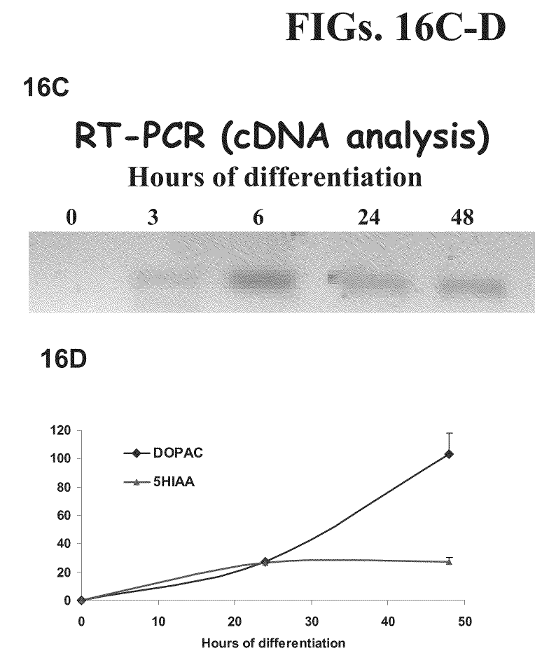

Thus, according to another embodiment of the present invention, the population of cells utilized by the present invention is a mixed population of cells which includes two or more different neurotransmitter producing cells aimed to provide a balanced neurotransmitter production. Preferably, the mixed population of cells includes dopaminergic as well as serotoninergic cells such as those illustrated in FIGS. 16A-D.

As is mentioned hereinabove, the cells utilized by this aspect of the present invention are preferably capable of controllable synthesis of the neurotransmitter.

Several approaches can be utilized to generate cells which are capable of such controlled synthesis.

Preferably, cell suitable for neuronal transplantation are harvested or generated as described hereinabove and are genetically modified to enable controllable expression of a neurotransmitter.

Genetic modification is preferably effected by transforming such cells with an expression construct which is designed for controllable expression of an enzyme participating in neurotransmitter synthesis.

The expression construct of the present invention preferably includes a polynucleotide sequence encoding an enzyme participating in synthesis of the neurotransmitter, whereas the expression construct is designed such that the polynucleotide expression is regulated via exposure to an agent or condition. Controllable expression of an enzyme participating in neurotransmitter synthesis can be effected by utilizing an expression construct which includes a polynucleotide sequence encoding the enzyme participating in neurotransmitter synthesis positioned under the transcriptional control of a promoter or regulatory which can be switched “on” (induced) or “off” (suppressed).

The expression construct can be designed as a gene knock-in construct in which case it will lead to genomic integration of construct sequences, or it can be designed as an episomal expression vector.

In any case, the expression construct can be generated using standard ligation and restriction techniques, which are well known in the art (see Maniatis et al., in: Molecular Cloning: A Laboratory Manual, Cold Spring Harbor Laboratory, New York, 1982). Isolated plasmids, DNA sequences, or synthesized oligonucleotides are cleaved, tailored, and religated in the form desired.

Polynucleotide sequences which can be utilized in the expression construct of the present invention are described in various databases such as that maintained by the national resource for molecular biology information (http://wwwdotncbidotnlmdotnihdotgov/). Examples include sequences set forth in GenBank Accession Nos. NM_{—}000360 (encoding tyrosine hydroxylase); NM_{—}000790 (encoding DOPA decarboxylase); NM_{—}000161 (encoding GTP cyclohydrolase I); NM_{—}000787 (encoding dopamine β-hydroxylase); NM_{—}002686 (encoding glutamate decarboxylase); NM_{—}003450 (encoding tryptophane-5 monooxygenase) and NM_{—}020549 (encoding choline acetyltransferase).

Promoters suitable for use with the present invention are preferably response elements capable for directing transcription of the polynucleotide sequence so as to confer regulatable synthesis of the neurotransmitter. A suitable response element can be, for example, a tetracycline response element (such as described by Gossen and Bujard (Proc. Natl. Acad. Sci. USA 89:5547-551, 1992); an ectysone-inducible response element (No D et al., Proc Natl Acad Sci USA. 93:3346-3351, 1996) a metal-ion response element such as described by Mayo et al. (Cell. 29:99-108, 1982); Brinster et al. (Nature 296:39-42, 1982) and Searle et al. (Mol. Cell. Biol. 5:1480-1489, 1985); a heat shock response element such as described by Nouer et al. (in: Heat Shock Response, ed. Nouer, L., CRC, Boca Raton, Fla., pp 167-220, 1991); or a hormone response element such as described by Lee et al. (Nature 294:228-232, 1981); Hynes et al. (Proc. Natl. Acad. Sci. USA 78:2038-2042, 1981); Klock et al. (Nature 329:734-736, 1987); and Israel and Kaufman (Nucl. Acids Res. 17:2589-2604, 1989). Preferably the response element is an ectysone-inducible response element, more preferably the response element is a tetracycline response element.

The expression construct of the present invention may also include one or more enhancers. Enhancer elements can stimulate transcription up to 1,000 fold from linked homologous or heterologous promoters. Enhancers are active when placed downstream or upstream from the transcription initiation site. Many enhancer elements derived from viruses have a broad host range and are active in a variety of tissues. For example, the SV40 early gene enhancer is suitable for many cell types. Other enhancer/promoter combinations that are suitable for the present invention include those derived from polyoma virus, human or murine cytomegalovirus (CMV), the long term repeat from various retroviruses such as murine leukemia virus, murine or Rous sarcoma virus and HIV. See, Enhancers and Eukaryotic Expression, Cold Spring Harbor Press, Cold Spring Harbor, N.Y. 1983, which is incorporated herein by reference.

Polyadenylation sequences can also be added to the expression construct in order to increase the translation efficiency of the enzyme expressed from the expression construct of the present invention. Two distinct sequence elements are required for accurate and efficient polyadenylation: GU or U rich sequences located downstream from the polyadenylation site and a highly conserved sequence of six nucleotides, AAUAAA, located 11-30 nucleotides upstream. Termination and polyadenylation signals that are suitable for the present invention include those derived from SV40.

In addition to the elements already described, the expression construct of the present invention may typically contain other specialized elements intended to increase the level of expression of cloned polynucleotides or to facilitate the identification of cells that carry the recombinant DNA. For example, a number of animal viruses contain DNA sequences that promote the extra chromosomal replication of the viral genome in permissive cell types. Plasmids bearing these viral replicons are replicated episomally as long as the appropriate factors are provided by genes either carried on the plasmid or with the genome of the host cell.

The expression construct may or may not include a eukaryotic replicon. If a eukaryotic replicon is present, then the vector is amplifiable in eukaryotic cells using the appropriate selectable marker. If the construct does not comprise a eukaryotic replicon, no episomal amplification is possible. Instead, the recombinant DNA integrates into the genome of the engineered cell, where the promoter directs expression of the desired polynucleotide.

The expression construct of the present invention can further include additional polynucleotide sequences that allow, for example, the translation of several proteins from a single mRNA such as an internal ribosome entry site (IRES) and sequences for genomic integration of the promoter-chimeric polypeptide. For example a single expression construct can be designed and co-express two distinct enzymes which participate in a neurotransmitter synthesis, such as the enzymes tyrosine hydroxylase and DOPA decarboxylase which participate in dopamine synthesis.

Examples for mammalian expression constructs include, but are not limited to, pcDNA3, pcDNA3.1(+/−), pGL3, pZeoSV2(+/−), pSecTag2, pDisplay, pEF/myc/cyto, pCMV/myc/cyto, pCR3.1, pSinRep5, DH26S, DHBB, pNMT1, pNMT41, pNMT81, which are available from Invitrogen, pCI which is available from Promega, pMbac, pPbac, pBK-RSV and pBK-CMV which are available from Strategene, pTRES which is available from Clontech, and their derivatives.

Expression constructs containing regulatory elements from eukaryotic viruses such as retroviruses can also be used by the present invention. SV40 vectors include pSVT7 and pMT2. Vectors derived from bovine papilloma virus include pBV-1MTHA, and vectors derived from Epstein Bar virus include pHEBO, and p2O5. Other exemplary vectors include pMSG, pAV009/A⁺, pMTO10/A⁺, pMAMneo-5, baculovirus pDSVE, and any other vector allowing expression of proteins under the direction of the SV-40 early promoter, SV-40 later promoter, metallothionein promoter, murine mammary tumor virus promoter, Rous sarcoma virus promoter, polyhedrin promoter, or other promoters shown effective for expression in eukaryotic cells.

Viruses are specialized infectious agents that have evolved, in many cases, to elude host defense mechanisms. Typically, viruses infect and propagate in specific cell types. The targeting specificity of viral vectors utilizes its natural specificity to specifically target predetermined cell types and thereby introduce a recombinant gene into the infected cell. Thus, the type of vector used by the present invention will depend on the cell type transformed. The ability to select suitable vectors according to the cell type transformed is well within the capabilities of the ordinary skilled artisan and as such no general description of selection consideration is provided herein. For example, bone marrow cells can be targeted using the human T cell leukemia virus type I (HTLV-I).

Recombinant viral vectors are useful for in vivo expression of transgenic polynucleotides since they offer advantages such as lateral infection and targeting specificity. Lateral infection is inherent in the life cycle of, for example, retrovirus and is the process by which a single infected cell produces many progeny virions that bud off and infect neighboring cells. The result is that a large area becomes rapidly infected, most of which was not initially infected by the original viral particles. This is in contrast to vertical-type of infection in which the infectious agent spreads only through daughter progeny. Viral vectors can also be produced that are unable to spread laterally. This characteristic can be useful if the desired purpose is to introduce a specified gene into only a localized number of targeted cells.

As described in the Examples section which follows, the cells of the present invention can also be transformed with an expression construct, or a construct system, which includes a first polynucleotide sequence which is regulated by a transactivator positioned under the transcriptional control of a second regulatory sequence. In such an expression scheme, the transactivator is capable of activating the first regulatory sequence to direct transcription of the first polynucleotide sequence in absence of the agent.

Preferably, the first polynucleotide sequence of the expression construct, or construct system, includes a sequence encoding an enzyme participating in a synthesis of a neurotransmitter which is operably linked to an ecdysone-responsive promoter such as described by No et al. (Proc Natl. Acad. Sci. USA. 93:3346-3351, 1996).

More preferably, the first polynucleotide sequence of the expression construct, or construct system, includes a sequence encoding an enzyme participating in a synthesis of a neurotransmitter which is operably linked to a tetracycline control element such as described by Gossen and Bujard (Proc. Natl. Acad. Sci. USA 89:5547-551, 1992). See Example 15 of the Examples section which follows for further details.

The transactivator is preferably a tetracycline controlled transactivator such as described by Gossen and Bujard (Proc. Natl. Acad. Sci. USA 89:5547-551, 1992) and may be operably linked to a human neuron-specific promoter such as the enolase promoter. See Example 15 of the Examples section which follows for further details.

Various methods can be used to introduce the expression construct of the present invention into mammalian cells. Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et al. [Biotechniques 4 (6): 504-512, 1986] and include, for example, stable or transient transfection, lipofection, electroporation and infection with recombinant viral vectors. In addition, see U.S. Pat. Nos. 5,464,764 and 5,487,992 for positive-negative selection methods.

Once transformed cells are generated, they are tested (in culture) for their ability to synthesize a functional neurotransmitter in response to an external signal (e.g., presence or absence of an agent). Preferably, the neurotransmitter concentration is comparatively (in presence vs absence of the agent) analyzed using standard chemical analytical methods such as, for example, HPLC, ELISA or GC-MS. Alternatively the cultures are comparatively analyzed for expression of the recombinant enzyme (e.g., tyrosine hydroxylase), using biochemical analytical methods such as immunoassays, Western blot and Real-time PCR using the procedures such as described in Examples 7 of the Examples section which follows, or by enzyme activity bioassays.

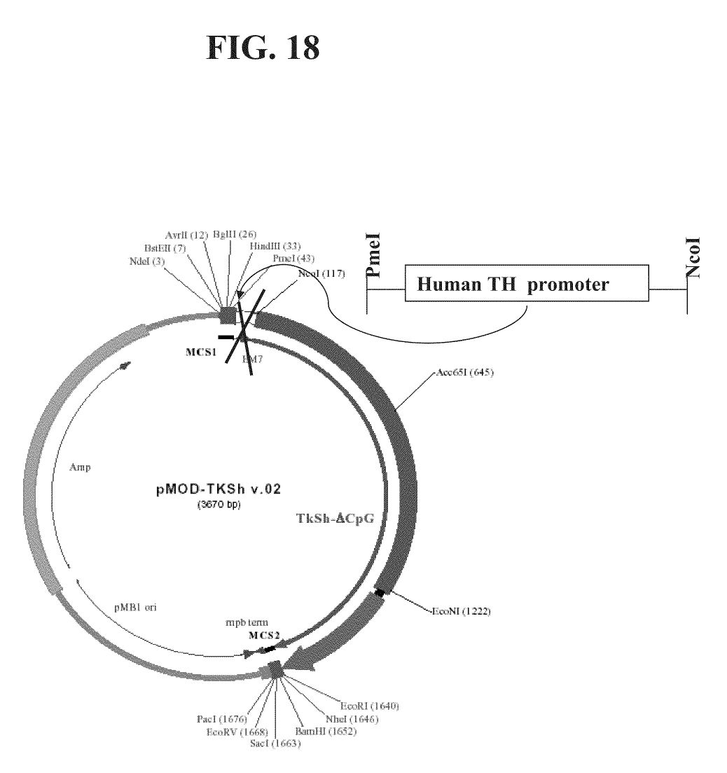

It will be appreciated that in cases where cells capable of endogenously producing a neurotransmitter are selected for use with the present invention (e.g., neuronal-induced BMSc) such cells are preferably genetically manipulated such as to delete or mutate endogenous coding sequences of enzymes participating in the neurotransmitter synthesis (e.g., endogenous tyrosine hydroxylase). Such genetic manipulation can be effected by, for example, by employing gene knock-out or site directed mutation techniques and vectors such as those described by Galli-Taliadoros et al. (J Immunol Methods 181:1-15, 1995) and Harris and Ford (Pharmacogenomics. 1:433-43, 2000). Alternatively, cells capable of endogenously producing a neurotransmitter can be eliminated by exposure to nerotoxins (e.g., MPTP) or by transformation with a suicide vector, such as illustrated in FIG. 18.

Deletion of endogenous sequences can be combined with knock-in of exogenous enzyme coding sequences (such as those described above) such that cells simultaneously lose the ability to endogenously synthesize neurotransmitters and acquire such an ability (regulatable) through genomic integration of exogenous sequences which encode the enzyme positioned under the transcriptional control of a controllable regulatory sequence.

Alternatively, such cells can also be genetically manipulated such that endogenous enzyme coding sequences are brought under control of a regulatable promoter sequence. Such manipulation can be achieved by replacing the endogenous promoter sequence of the enzyme (e.g., the TH promoter sequence) via gene knock-in of a regulatable promoter sequence.

Optionally, the cells of the present invention are transformed so as to acquire resistance to cell death occurring during brain transplantation. It has been found that cells implanted in brain tissue may undergo apoptosis triggered by hypoxia, hypoglycemia, mechanical trauma, free radicals, growth factor depravation, and excessive extracellular concentrations of excitatory amino acids in the host brain (Brundin et al. (Cell Transplant. 9:179-195, 2000). Under circumstances where the risk of apoptosis-induced cell death is high, the cells of the present invention can be transformed with a polynucleotide encoding an apoptosis inhibiting polypeptide such as, for example, the human bcl-2 gene (Adams and Cory, Science 281:1322-1326, 1998). The polypeptide can be expressed under the control of a constitutive promoter such as described hereinabove, or preferably, under a control of a neuronal tissue-specific promoter such as, for example the human neuron-specific enolase (NSE) promoter as described by Levy et al. (Journal of Molecular Neuroscience 21:121-132, 2003).

Neurotransmitter release may be further controlled by providing to the subject PUFA following transplantation of the cells of the present invention as further detailed below.

The cells of the present invention can be administered to the treated individual using a variety of transplantation approaches, the nature of which depends on the site of implantation.

The term or phrase “transplantation”, “cell replacement” or “grafting” are used interchangeably herein and refer to the introduction of the cells of the present invention to target tissue. The cells can be derived from the recipient or from an allogeneic or xenogeneic donor.

The cells can be grafted into the central nervous system or into the ventricular cavities or subdurally onto the surface of a host brain. Conditions for successful transplantation include: (i) viability of the implant; (ii) retention of the graft at the site of transplantation; and (iii) minimum amount of pathological reaction at the site of transplantation. Methods for transplanting various nerve tissues, for example embryonic brain tissue, into host brains have been described in: “Neural grafting in the mammalian CNS”, Bjorklund and Stenevi, eds. (1985); Freed et al., 2001; Olanow et al., 2003). These procedures include intraparenchymal transplantation, i.e. within the host brain (as compared to outside the brain or extraparenchymal transplantation) achieved by injection or deposition of tissue within the host brain so as to be opposed to the brain parenchyma at the time of transplantation.

Intraparenchymal transplantation can be effected using two approaches: (i) injection of cells into the host brain parenchyma or (ii) preparing a cavity by surgical means to expose the host brain parenchyma and then depositing the graft into the cavity. Both methods provide parenchymal deposition between the graft and host brain tissue at the time of grafting, and both facilitate anatomical integration between the graft and host brain tissue. This is of importance if it is required that the graft becomes an integral part of the host brain and survives for the life of the host.

Alternatively, the graft may be placed in a ventricle, e.g. a cerebral ventricle or subdurally, i.e. on the surface of the host brain where it is separated from the host brain parenchyma by the intervening pia mater or arachnoid and pia mater. Grafting to the ventricle may be accomplished by injection of the donor cells or by growing the cells in a substrate such as 3% collagen to form a plug of solid tissue which may then be implanted into the ventricle to prevent dislocation of the graft. For subdural grafting, the cells may be injected around the surface of the brain after making a slit in the dura. Injections into selected regions of the host brain may be made by drilling a hole and piercing the dura to permit the needle of a microsyringe to be inserted. The microsyringe is preferably mounted in a stereotaxic frame and three dimensional stereotaxic coordinates are selected for placing the needle into the desired location of the brain or spinal cord. The cells may also be introduced into the putamen, nucleus basalis, hippocampus cortex, striatum, substantia nigra or caudate regions of the brain, as well as the spinal cord.

The cells may also be transplanted to a healthy region of the tissue. In some cases the exact location of the damaged tissue area may be unknown and the cells may be inadvertently transplanted to a healthy region. In other cases, it may be preferable to administer the cells to a healthy region, thereby avoiding any further damage to that region. Whatever the case, following transplantation, the cells preferably migrate to the damaged area. As described in Example 18, damaged substantia nigra in the rat model of Parkinson's has the ability to attract BMSC to that region.

For transplanting, the cell suspension is drawn up into the syringe and administered to anesthetized transplantation recipients. Multiple injections may be made using this procedure.

The cellular suspension procedure thus permits grafting of the cells to any predetermined site in the brain or spinal cord, is relatively non-traumatic, allows multiple grafting simultaneously in several different sites or the same site using the same cell suspension, and permits mixtures of cells from different anatomical regions. Multiple grafts may consist of a mixture of cell types, and/or a mixture of transgenes inserted into the cells. Preferably from approximately 10⁴ to approximately 10⁸ cells are introduced per graft.

For transplantation into cavities, which may be preferred for spinal cord grafting, tissue is removed from regions close to the external surface of the central nerve system (CNS) to form a transplantation cavity, for example as described by Stenevi et al. (Brain Res. 114:1-20., 1976), by removing bone overlying the brain and stopping bleeding with a material such a gelfoam. Suction may be used to create the cavity. The graft is then placed in the cavity. More than one transplant may be placed in the same cavity using injection of cells or solid tissue implants. Preferably, the site of implantation is dictated by the type of neurotransmitter being synthesized by the cells of the present invention. For example, dopaminergic cells are preferably implanted in the sabstantia nigra of a Parkinson's patient.

Prior to the aspiration of bone marrow cells and following their transplantation, the subject may be administered with a fatty acid. As discussed above, inclusion of fatty acids in the differentiation medium of the transplanted cells promotes neural differentiation. Without being bound to any theory, it is envisaged that administration of fatty acids prior to the aspiration of bone marrow cells and following their transplantation may aid in neuronal differentiation and maintaining the cells to be in a neuronally differentiated state. The fatty acids may be ingested as part of a fat-rich meal (e.g. by eating a quantity of food which comprises a high PUFA content, such as margarine). It will be further appreciated that the agents of the present invention may also be provided as food additives.

The phrase “food additive” [defined by the FDA in 21 C.F.R. 170.3(e)(1)] includes any liquid or solid material intended to be added to a food product. This material can, for example, include an agent having a distinct taste and/or flavor or a physiological effect (e.g., vitamins). The food additive composition of the present invention can be added to a variety of food products.

As used herein, the phrase “food product” describes a material consisting essentially of protein, carbohydrate and/or fat, which is used in the body of an organism to sustain growth, repair and vital processes and to furnish energy. Food products may also contain supplementary substances such as minerals, vitamins and condiments. See Merriani-Webster's Collegiate Dictionary, 10th Edition, 1993. The phrase “food product” as used herein further includes a beverage adapted for human or animal consumption.

A food product containing the food additive of the present invention can also include additional additives such as, for example, antioxidants, sweeteners, flavorings, colors, preservatives, nutritive additives such as vitamins and minerals, amino acids (i.e. essential amino acids), emulsifiers, pH control agents such as acidulants, hydrocolloids, antifoams and release agents, flour improving or strengthening agents, raising or leavening agents, gases and chelating agents, the utility and effects of which are well-known in the art.

Thus, for example, the subject may be administered as an article of manufacture which includes the PUFA and is identified for treating a neurodegenerative disease such as Parkinson's Disease following transplantation of the cells of the present invention. Preferably, the administration of the fatty acids continues for one day, even more preferably for one week and even more preferably for one month.

The cells of the present invention may be co-administered with therapeutic agents useful in treating neurodegenerative disorders, such as growth factors, e.g. nerve growth factor and/or glial cell line-derived neurotrophic factor (GDNF); gangliosides; antibiotics, neurotransmitters, neurohormones, toxins, neurite promoting molecules; and antimetabolites and precursors of these molecules such as L-DOPA.

Following transplantation, the cells of the present invention preferably survive in the diseased area for a period of time (e.g. at least 6 months), such that a therapeutic effect is observed. As described in Example 17, mouse BMSC were shown to survive longer in the 6-OHDA lesioned mouse brain hemisphere as opposed to the non-lesioned hemisphere.

Following transplantation, the treated individual is carefully and continuously monitored for the level of neurotransmitter released by the implanted cells. The neurotransmitter level is preferably estimated indirectly by using clinical tests suitable for diagnosing the neurodegenerative disorder. For example, the release of dopamine by implanted cells in a Parkinson's disease patient can be estimated using clinical diagnosis tests for Parkinson's disease such as described, for example in Adker, C. H. and Ahlskog, J. E eds. (“Parkinson's Disease and Movement Disorders, Diagnosis and Treatment Guidelines for the Practicing Physician, Humana Press”, New Jersey, 2000). Based on monitored indications, the neurotransmitter release rate is adjusted by administering to the individual, or withholding from the individual, an agent capable of regulating synthesis of the neurotransmitter in the implanted cells. The agent may be any molecule capable of upregulating or downregulating the expression of an enzyme participating in the synthesis of the neurotransmitter, such as described hereinabove.

The agent can be administered directly to the individual or as a part (active ingredient) of a pharmaceutical composition.

As used herein a “pharmaceutical composition” refers to a preparation of one or more of the active ingredients or agents described herein with other chemical components such as physiologically suitable carriers and excipients. The purpose of a pharmaceutical composition is to facilitate administration of a compound to an organism.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, Pa., latest edition, which is incorporated herein by reference.

For any preparation used in the methods of the invention, the therapeutically effective amount or dose can be estimated initially from in vitro and cell culture assays. Preferably, a dose is formulated in an animal model to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. For example, 6-OHDA-lesioned mice may be used as animal models of Parkinson's. Survival and rotational behavior of the mice may be analyzed (as in Example 16). The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition, (see e.g., Fingl, et al., 1975, in “The Pharmacological Basis of Therapeutics”, Ch. 1 p. 1). For example, Parkinson's patient can be monitored symptomatically for improved motor functions indicating positive response to treatment, and for runaway diskinesis symptoms indicating an excessive dopamine expression.

The agent can be administered to the patient in various ways, including but not limited to oral administration, parenteral administration, intrathecal administration, intraventricular administration and intranigral application. The exact formulation, route of administration and dosage can be chosen by the individual physician in view of the patient's condition. (see e.g., Fingl, et al., 1975, in “The Pharmacological Basis of Therapeutics”, Ch. 1 p. 1).

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

The pharmaceutical composition described herein may be formulated for parenteral administration, e.g., by bolus injection or continuos infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Dosage amount and interval may be adjusted individually to levels of the active ingredient which are sufficient to effectively regulate the neurotransmitter synthesis by the implanted cells. Dosages necessary to achieve the desired effect will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or diminution of the disease state is achieved.

The amount of a composition to be administered will, of course, be dependent on the individual being treated, the severity of the affliction, the manner of administration, the judgment of the prescribing physician, etc. The dosage and timing of administration will be responsive to a careful and continuous monitoring of the individual changing condition. For example, a treated Parkinson's patient will be administered with an amount of agent which is sufficient to promote, or suppress, dopamine synthesis to the level desired, based on the monitoring indications.

Hence, the invention provides novel nucleic acid constructs, construct systems, cells and methods of cell therapy of neurodegenerative diseases which is effective, safe and clinically practical.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984); “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996) and Parfitt et al. (1987). Bone histomorphometry: standardization of nomenclature, symbols, and units. Report of the ASBMR Histomorphometry Nomenclature Committee. J Bone Miner Res 2 (6), 595-610; all of which are incorporated by reference as if fully set forth herein. Other general references are provided throughout this document. The procedures therein are believed to be well known in the art and are provided for the convenience of the reader. All the information contained therein is incorporated herein by reference.

### Example 1

### Isolation and Culturing of Human Bone-Marrow Stromal Cells (hBMSc)

Methods:

Proliferation Culture:

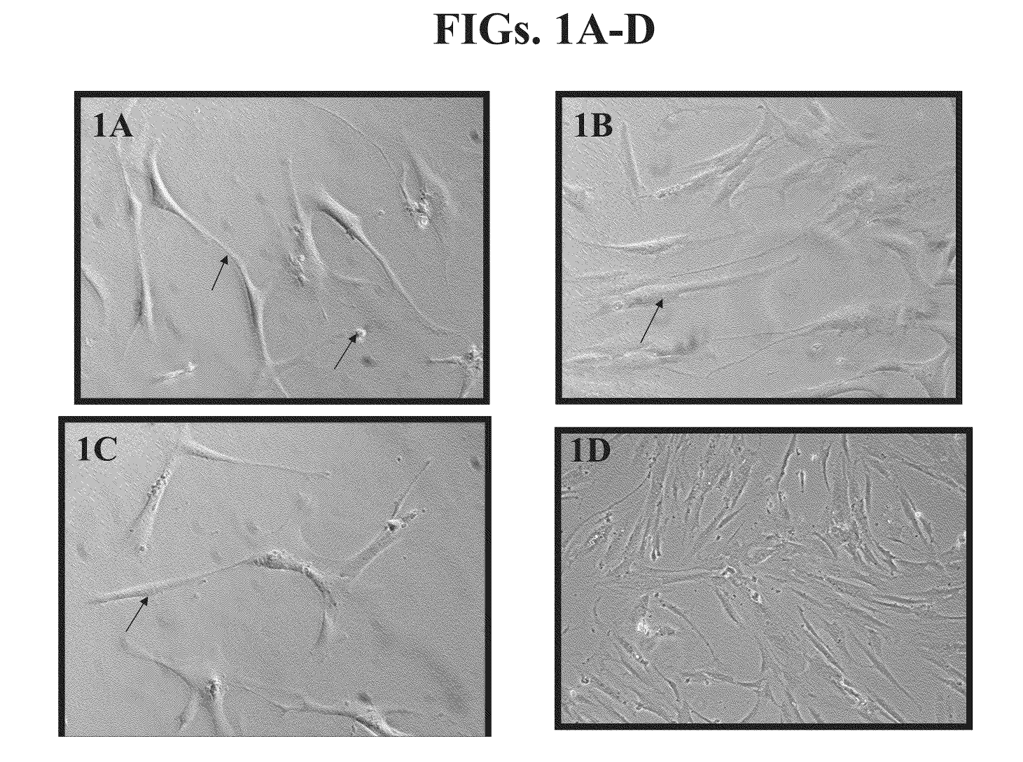

Bone marrow aspirates (10 ml) were obtained from iliac crest of healthy human donors with informed consent. Mononuclear cells were isolated by centrifugation through a Ficoll density gradient (Histopaque®-1077, Sigma, St. Louis, Mo.) or in UNISEP-MAXI tubes (Novamed, Jerusalem, Israel) on the basis of density gradient. The mononuclear cell layer was recovered from the gradient interface, washed with HBSS and centrifuged at 2000 g for 20 min at room temperature), and cells were plated in 75-cm² polystyrene plastic tissue-culture flasks (Corning, Corning, N.Y.) in a “proliferation medium” [Dulbecco's modified eagle medium (DMEM; Biological Industries); 100 μg/ml streptomycin, 100 U/ml penicillin, 12.5 units/ml nystatin (SPN; Biological Industries); 2 mM L-glutamine; 5% horse serum; 15% fetal calf serum (FCS; Biological Industries); 0.001% 2-β-mercaptoethanol (Sigma); 1× non-essential amino acids; 10 ng/ml human epidermal growth factor (EGF)]. The cells were incubated for two days at 37° C. in a humidified 5% CO2 incubator in normal or in low oxygen (O₂-3%, N₂-72%), and non-adherent cells were then discarded. The remaining plastic-adherent cells were washed twice with Dulbecco's phosphate-buffered saline (PBS; Biological Industries), and fresh growth medium was added. The medium was replaced every 3 or 4 days. Cells grew to 80-90% confluency within 15 days and appeared round or spindle shaped (FIG. 1A) or a flat shaped (FIGS. 1B-D).

Flow Cytometry:

Following 15 days under proliferative culturing conditions, the cells were harvested and suspended in 0.05% trypsin and 25 mM EDTA in phosphate-buffered saline (PBS). The cells, in solution at a concentration of 0.5×10⁶ cells/ml, were stained with antibodies specific against the cell surface markers CD45, CD5, CD20, CD11b and CD34 (associated with lympho-hematopoietic cells; Becton-Dickinson) for 20 min with an empirically determined amount of each antibody, generally 10 to 20 μl. The antibody-labeled cells were thoroughly washed with two volumes of PBS and fixed in flow buffer (1% paraformaldehyde, 0.1% sodium azide, and 0.5% bovine serum albumin in PBS). The washed cells were analyzed by a FACSCalibur™ flow cytometer (Becton Dickinson), equipped with an argon ion laser, adjusted to an excitation wavelength of 488 nm, and by collecting 10,000 events with the CELLQuest™ software program (Becton Dickinson).

Results:

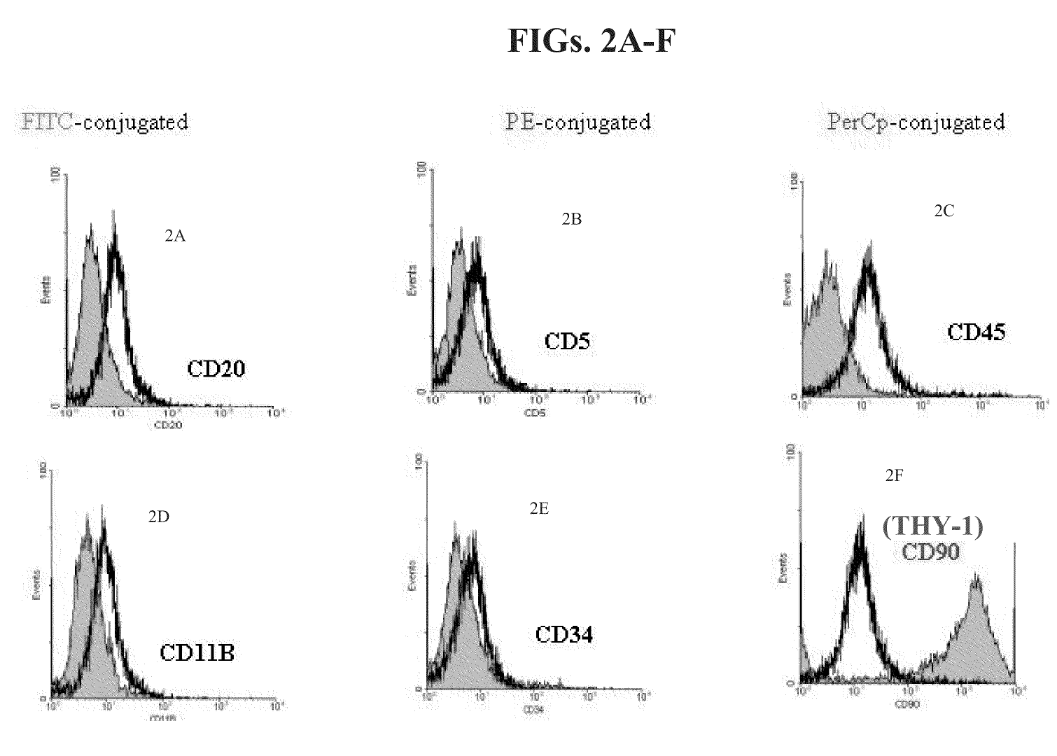

The cultured hBMSc did not express any of the surface markers associated with lympho-hematopoietic cells (i.e., CD45, CD5, CD20, CD11b and CD34), but rather expressed the CD90 surface marker (Thy-1), which is indicative of synaptogenesis and for mesencymal stem cells (FIGS. 2A-2F).

### Example 2

### In Vitro Differentiation of Human Bone-Marrow Stromal Cells (hBMSc)

Methods:

Differentiation Cultures:

hBMSc were cultured in the “proliferation medium” (described in Example 1 hereinabove) for up to three months prior to differentiation induction. Plastic-adherent cells were then transferred to an “additional differentiation medium”. Following 24-48 hr incubation at 37° C. the cells were transferred to a “differentiating medium” and incubated at 37° C. for 12-96 hr. For long-term differentiation medium see example 5.

TABLE 1
Culture media used to induce neuronal differentiation of hBMSc

| | |
|---|---|
| Stage 1: | Dulbecco's modified eagle medium (DMEM; |
| Proliferation | without HEPES); 100 μg/ml streptomycin, |
| medium | 100 U/ml penicillin, 12.5 units/ml nystatin (SPN); |
| (weeks) | 2 mM L-glutamine; 15% fetal calf serum (FCS); |
| | 0.001% 2-β-mercaptoethanol; Non-essential |
| | amino acids X1; 10 ng/ml human epidermal growth |
| | factor (EGF) |
| Stage 2: | DMEM/F12 (without HEPES); 2 mM L-glutamine; |
| Additional | SPN; 10% FCS/FBS; *N2 supplement; 10 ng/ml |
| Differentiation | human basic fibroblast growth factor (bFGF); |
| medium | 10 ng/ml EGF; 40 μM arachidonic acid; 10-40 μM |
| (24-48 hr) | docosahexaenoic acid (DHA); 40 μM α-tocopherol |
| Stage 3: | DMEM; 2 mM L-glutamine; SPN; *N2 supplement; |
| Differentiation | 1 mM dibutyryl cyclic AMP (dbcAMP); 0.5 mM |
| medium | isobutylmethlxanthine (IBMX); 1 μM all-trans-retinoic |
| (12-96 hr) | acid; 200 μM butylated hydroxyanisole (BHA); |
| | 20-40 μM arachidonic acid; 40 μM α-tocopherol |

*N2 supplement: insulin 25 μg/ml; progesterone 20 nM; putrescin 100 μM; selenium 30 nM; transferrin 100 μg/ml.

Proliferation Assessment:

hBMSc were suspended in the “additional differentiation medium” and in the “differentiation medium”, dispensed in 96-well microtiter plates (100 μl/well) and incubated for 16 and 39 hr at 37° C. The cultures were then supplemented with 10 μCi/ml ³H-thymidine and incubated for four additional hours. Cells were then harvested by suspended in 0.05% trypsin and 25 mM EDTA in phosphate-buffered saline (PBS), and analyzed with a liquid scintillation counter to determine the level of ³H-thymidine incorporation in the cells (indicative of proliferation activity).

Results:

Plastic-adherent cells exhibited neuronal-like spindle body shape with long branching processes that appeared as early as three hours post differentiation induction and continued to appear 72 h following differentiation induction (FIGS. 3A-F).

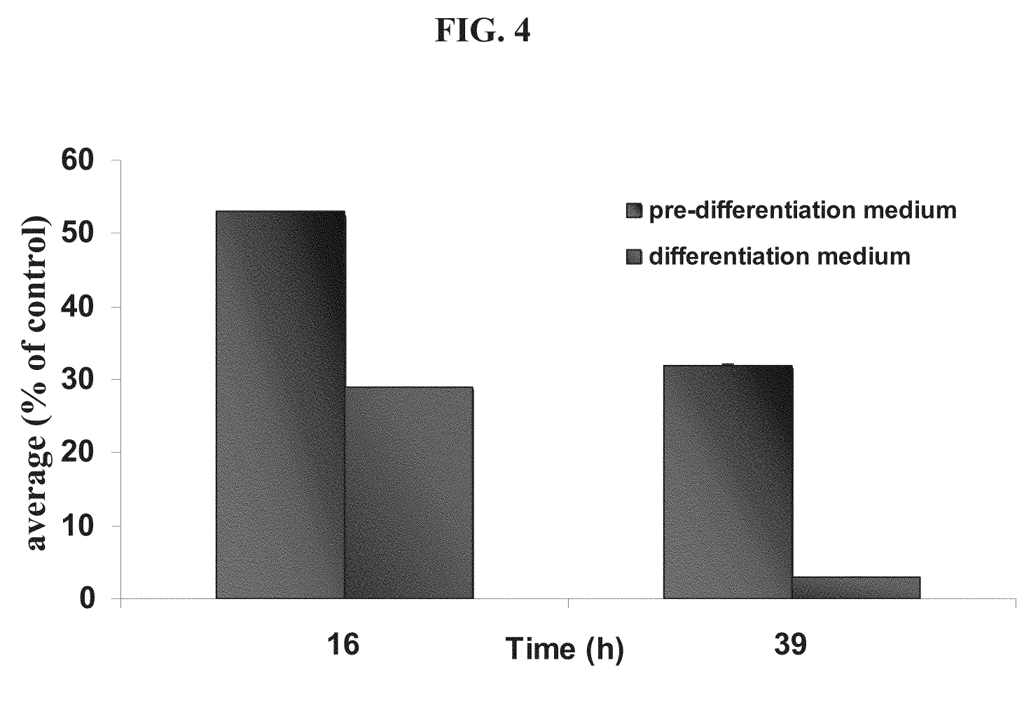

³H-thymidine incorporation was substantially reduced in the differentiated cells (FIG. 4) thus indicating that proliferation was attenuated in the neuronal-like differentiated hBMSc.

### Example 3

### Identification of Neuronal Transcripts in Differentiating hBMSc

Methods

RT-PCR:

hBMSc which were incubated in the “proliferation medium” or in the “differentiation medium” (see Examples 1-2 hereinabove) for 3-72 hours at 37° C. Total RNA was extracted from the hBMSc by using the guanidine isothiocyanate method as described by Chomczynski & Sacchi (1987). In addition total RNA was extracted from fresh human lymphocytes (from donor) using the RNA isolated kit (Puregene Gentra, Manneapolis, USA). The RNA samples were separated on 1% agarose formaldehyde-denaturing gel electrophoreses to verify their integrity. For generating cDNA the RNA samples (0.5 μg) were mixed with RT-superscript II enzyme (10 units) contained in a reaction mixture [1.3 μM random primer, 1× Buffer (supplied by InvitroGene), 10 mM DTT, 20 μM dNTPs, and RNase inhibitor] and incubated at 25° C. for 10 min, 42° C. for 2 hours, 70° C. for 15 min and 95° C. for 5 min. The resulting cDNA samples were analyzed by PCR using the primers set forth by SEQ ID NOs: 1-2, 11-12, 21-22, 26-26 and 29-30 (see Table 1 below) and amplified under 35 cycles at 94° C. for 1 min, 55-58° C. for 1 min and 72° C. for 1 min.

TABLE 2
Upstream sense and downstream anti-sense primers from different exons for
detection of neuronal and dopaminergic transcripts in differentiated and non
differentiated hBMSc

| Human gene | | 5′ Primer | 3′ Primer | Product size |
|---|---|---|---|---|
| NCBI No. | Cellular Function | SEQ ID NO | SEQ ID NO | (bp) |
| CD90 (Thy-1) | May play a role during | 1 | 2 | 312 |
| NM_006288 | synaptogenesis | | | |
| Dopamine receptor | one of the five types (D1 to D5) | 3 | 4 | 159 |
| D2 (D2DR) | of receptors for dopamine | | | |
| NM_016574 | | | | |
| Dopamine | Amine transporter. Terminates | 5 | 6 | 253 |
| transporter (DAT) | the action of dopamine by its | | | |
| NM_001044 | high affinity sodium-dependent | | | |
| | reuptake into presynaptic | | | |
| | terminals. | | | |
| Aromatic L-amino | Catalyzes the decarboxylation | 7 | 8 | 250 |
| acid decarboxylase | of L-3,4- | | | |
| (AADC) | dihydroxyphenylalanine | | | |
| NM_000790 | (DOPA) to dopamine, | | | |
| | L-5-hydroxytryptophan to | | | |
| | serotonin and L-tryptophan to | | | |
| | tryptamine | | | |
| Glyceraldehyde-3- | Catalytic enzyme of glycolysis | 9 | 10 | 194 |
| phosphate | | | | |
| dehydrogenase | | | | |
| (GAPDH) | | | | |
| NM_002046 | | | | |
| Glypican 4 (GPC4) | Cell surface proteoglycan may | 11 | 12 | 386 |
| NM_001448 | be involved in the development | | | |
| | of CNS | | | |
| GTP | Tetrahydrobiopterin | 13 | 14 | 153 |
| cyclohydrolase 1 | biosynthesis | | | |
| (GTPCH1) | | | | |
| NM_000161 | | | | |
| *Necdin | Postmitotic neuron-specific | 15 | 16 | 394 |
| NM_002487 (* | growth suppressor | | | |
| Only one exone) | | | | |
| Neurite growth- | Extracellular matrix-associated | 17 | 18 | 359 |
| promoting factor 2 | protein that enhances axonal | | | |
| (NEGF2) | growth in perinatal cerebral | | | |
| X55110 | neurons. | | | |
| Nestin | Intermediate filament protein a | 19 | 20 | 398 |
| X65964 | predominant marker used to | | | |
| | describe stem and progenitor | | | |
| | cells in the mammalian CNS | | | |
| Neuron specific | Isozyme of the glycolytic | 21 | 22 | 356 |
| enolase | enzyme enolase is expressed in | | | |
| X13120 | all neuronal cell types | | | |
| Neurofilament | 200 kDa filaments slowly | 23 | 24 | 400 |
| heavy (NF-H) | transported within the axons | | | |
| NM_021076 | towards the synaptic terminals | | | |
| Neurofilament | 160 kDa filaments slowly | 25 | 26 | 366 |
| medium (NF-M) | transported within axons | | | |
| XM_005158 | towards the synaptic terminals | | | |
| Nuclear receptor | Dopaminergic transcription | 27 | 28 | 550 |
| related 1(Nurr1) | factor | | | |
| NM_006186 | | | | |
| Retinoic acid | Receptor for retinoic acid | 29 | 30 | 352 |
| receptor type α | | | | |
| (RA-R) | | | | |
| NM_000964 | | | | |
| Neurofilament | 68 kDa filaments slowly | 41 | 42 | 350 |
| light(NF-L) | transported within axons | | | |
| NM_006158 | towards the synaptic terminals | | | |
| Neurotrophic | Interacts with neurotrophins and | 43 | 44 | 352 |
| tyrosine kinase | mediates their function | | | |
| receptor type 2 | | | | |
| (TRK-2) | | | | |
| NM_006180 | | | | |
| Patched | Receptor for sonic hedgehog | 45 | 46 | 207 |
| homolog(PTCH) | | | | |
| NCBI: | | | | |
| NM_000264 | | | | |
| RET tyrosine | A cell-surface | 47 | 48 | 233 |
| kinase, | molecules that transduce signals | | | |
| NM_000323.2 | for cell growth and | | | |
| | differentiation. | | | |
| Smoothened (SMO) | Receptor for sonic hedgehog | 49 | 50 | 167 |
| NM_005631.2 | | | | |
| Vesicular | A synaptic vesicle monoamine | 51 | 52 | 189 |
| monoamine | transporter | | | |
| transporter 2 | | | | |
| (VMAT 2) | | | | |
| NM_003054.1 | | | | |
| Engrailed 1(En-1) | Implicated in the control of | 53 | 54 | 180 |
| NM_001426 | development | | | |
| Nurr-1 | a member of the steroid-thyroid | 55 | 56 | 240 |
| NM_006186.2 | hormone-retinoid receptor | | | |
| | superfamily | | | |
| Paired-like | a member of the RIEG/PITX | 57 | 58 | 175 |
| homeodomain | homeobox | | | |
| transcription factor | family | | | |
| 3 (PITX-3) | | | | |
| NM_005029.3 | | | | |
| Catechol-o- | catalyzes the transfer of a | 59 | 60 | 230 |
| methyltransferase | methyl group from S- | | | |
| (COMT) | adenosylmethionine to | | | |
| NM_000754.2 | catecholamines, including | | | |
| | the neurotransmitters dopamine, | | | |
| | epinephrine, and norepinephrine | | | |
| GTP | catalyzes the | 61 | 62 | 153 |
| cyclohydrolase-1 | conversion of GTP to D- | | | |
| (GCH) | erythro-7,8-dihydroneopterin | | | |
| NM_000161 | triphosphate, | | | |
| | the first and rate-limiting step in | | | |
| | tetrahydrobiopterin (BH4) | | | |
| | biosynthesis. | | | |
| Monoamine | Amine oxidase | 63 | 64 | 237 |
| oxidase B (MAO- | | | | |
| B) | | | | |
| \|NM_000898.2\| | | | | |
| Aldehyde | Enzyme that convert | 65 | 66 | 154 |
| dehydrogenase 1 | retinaldehde to retinoic acid | | | |
| (Aldh1): | specific at progenitor | | | |
| NM_000689.3 | dopaminergic cells | | | |

Northern Blot Analysis:

RNA samples extracted from hBMSc were size fractionated on 1% agarose gel supplemented with 3% formaldehyde and MOPS, and transferred to Duralon-UV™ membranes (Stratagene). The membranes were then hybridized overnight with purified ³²P-labeled probes for neuronal markers NEGF2 (neurite growth-promoting factor 2), NF-200 (neurofilament heavy), and NSE (neuron specific enolase). The hybridized membranes were washed several times, exposed to storage phosphor screen, autoradiographed by phosphorimager (Cyclone, Packard), stripped and rehybridized with a ³²P-labeled probe for GAPDH (Glyceraldehyde-3-phosphate dehydrogenase) to verify equal loading and transfer of RNA.

Real Time PCR:

Real-time quantitative PCR analysis of the neuronal marker NEGF2 was performed in a “Rotor-Gene DNA sample analysis system” version 4.6 (Corbett Research) using Sybergreen “PCR master mix” and the primers of SEQ ID NOs: 17-18. In addition, Real time PCR analysis of GADPH was performed for providing stimulated conditions for sample normalization using the primers of SEQ ID NOs: 9-10. The amplification protocol was 40 cycles of 95° C. for 15 sec, 55° C. for 40 sec, 72° C. for 40 sec and 77° C. for 20 sec. Stimulated conditions for sample normalization were applied by amplification of 18S rRNA. The amplification protocol was 80 cycles of 95° C. for 20 and 61° C. for 1 min. Quantification of gene expression relative to 18S rRNA was calculated by the protocol's ΔΔCT method and from standard curve method.

Results:

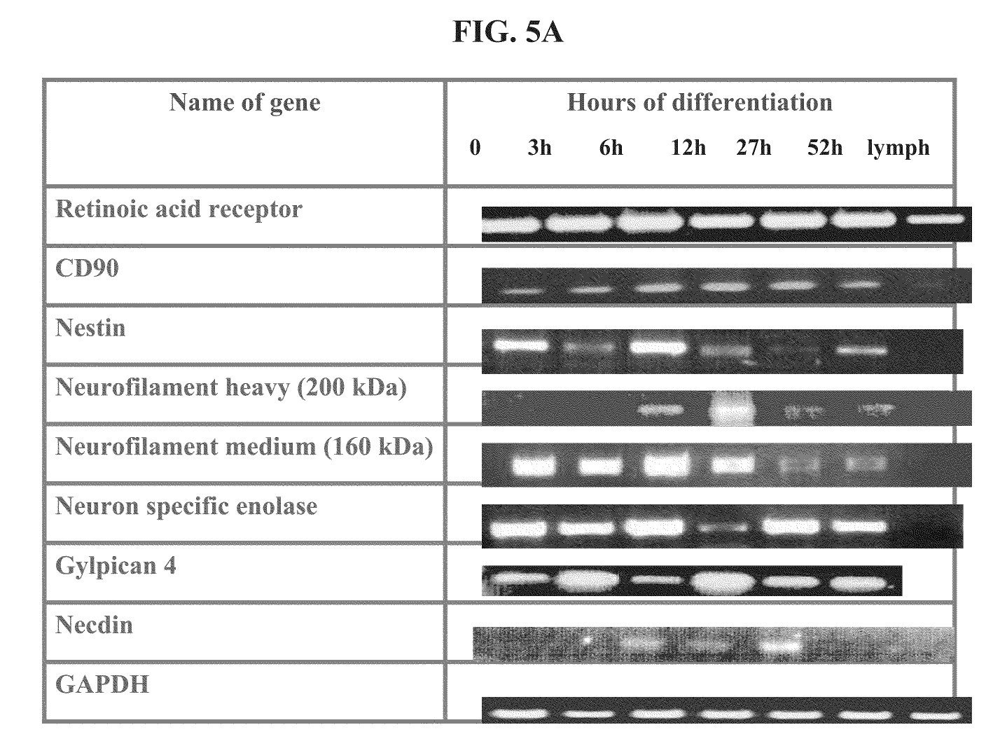

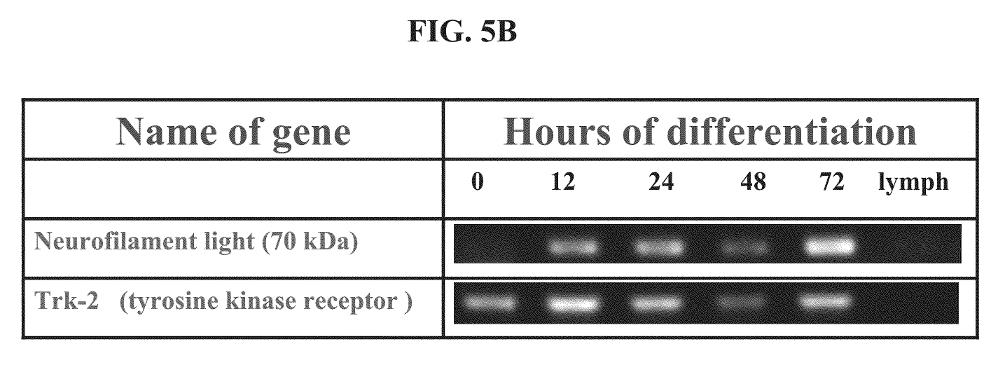

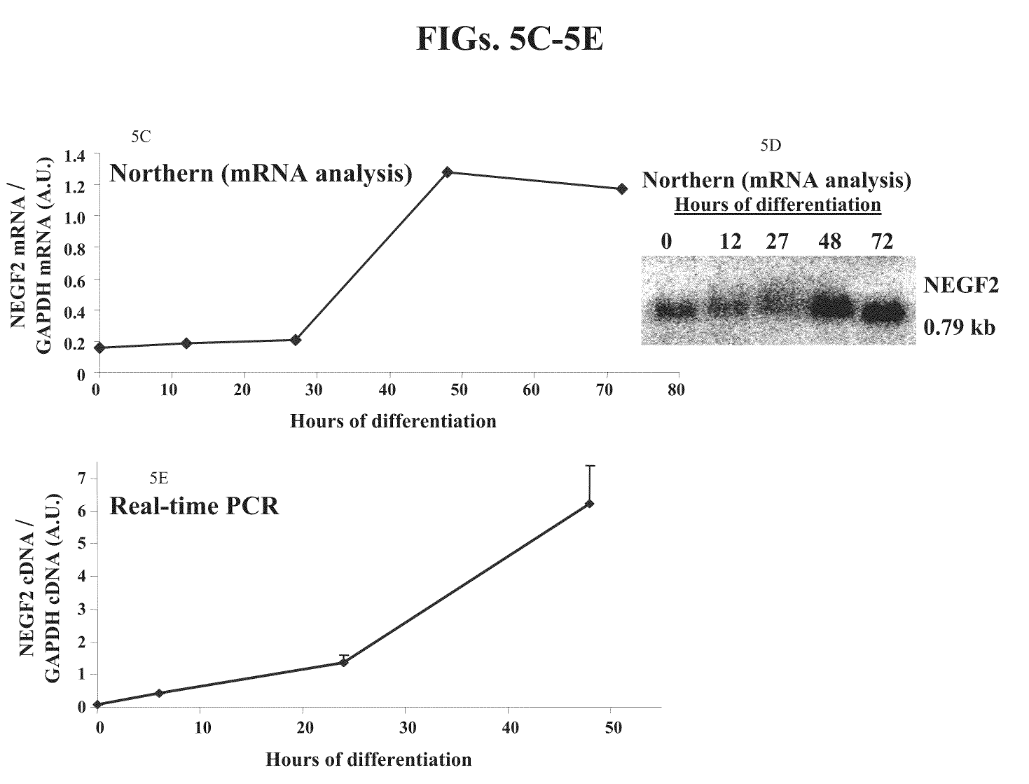

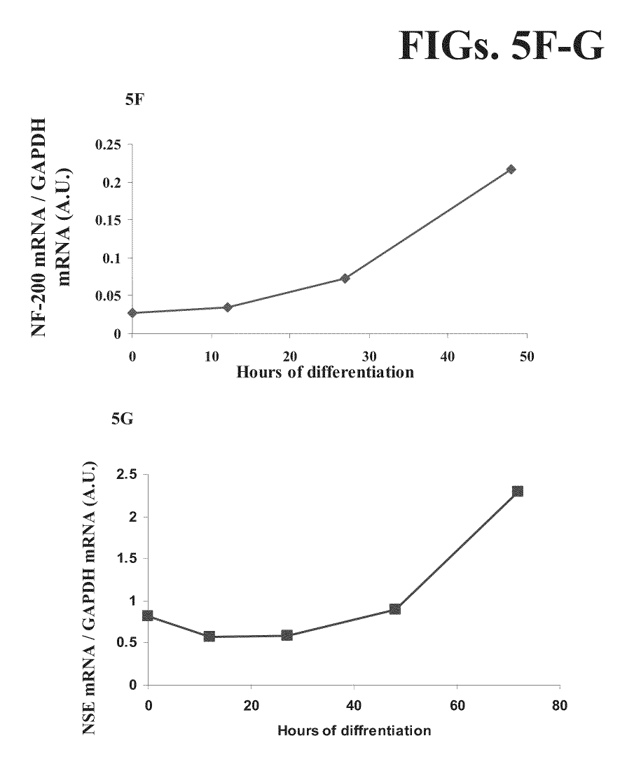

RT-PCR analysis indicates transcriptional expression of neuronal markers nestin, NSE, NF-M, CD90, RA-R, Trk-2 and GPC4 in both differentiated and non-differentiated hBMSc (FIGS. 5A and 5B). However, transcriptional expression of the neuronal markers NF-L, NF-H and necdin occurred only in the differentiated hBMSc (FIGS. 5A and 5B).

Real time PCR analysis shows a seven fold increase of NEGF2 mRNA in the differentiating cells, as compared with non-differentiated cells, following 50 hr incubation (FIG. 5E).

Northern blot analyses show that transcriptional expression of the neuronal markers NEGF2, (FIGS. 5C and 5D) NF-200 (FIG. 5F) and NSE (FIG. 5G) markedly increased in differentiating hBMSc.

### Example 4

### Identification of Neuronal Proteins in Differentiated hBMSc

Methods

Western Blot:

Fifty micrograms of protein extracts obtained from hBMSc were denatured in a sample buffer (62.5 mM Tris-HCl at pH 6.8, 10% glycerol, 2% SDS, 5% 2-β-mercaptoethanol, 0.0025% bromophenol blue (SIGMA), diluted 1:5 with the sample and boiled for 5 min. Each sample was loaded on a 12.5% SDS-polyacrylamide gel (Bio-Rad Laboratories), according to the manufacturer's instruction. Following electrophoresis, proteins were transferred to polyvinylidene difluride membrane (Bio-Rad Laboratories), followed by blocking with 5% nonfat milk in Tris-buffered saline (TSB 10 mM Tris at 7.5, 150 mM NaCl) with 0.1% Tween-20 (blocking solution). The membranes were probed overnight, at 4° C., with neuron markers-specific antibodies as described in Table 3 herein below. Following incubation, were the membranes were washed twice (15 min each) with blocking solution and once with TBS-T for 15 min, then exposed to horseradish peroxidase-conjugated secondary antibody as described in Table 3 herein below. The membranes were then washed twice (15 min each) with blocking solution and once in TBS-T for 15 min and were stained using the enhanced SuperSignal® chemiluminescent detection kit (Pierce) and exposed to medical X-ray film (Fuji Photo Film). Actin was used to evaluate and quantify the changes during the induction. Densitometry of the specific proteins bands was preformed by using VersaDoc® imaging system (Bio-Rad Laboratories) and Quantity One® software (Bio-Rad).

TABLE 3
Neuronal and dopaminergic marker-specific antibodies

| | | Dilution | Dilution | Dilution | |
|---|---|---|---|---|---|
| Specificity | Source | for IA* | for WB* | for FC* | Source |
| Actin | Mouse | | 1:1000 | | Chemicon |
| | | | | | International |
| α-Synuclein | Mouse | 1:100 | | | Zymed |
| β-tubulin III | Mouse | 1:400 | | 1:50 | Sigma |
| D2 Dopamine | Mouse | | | 1:50 | Santa-Cruz |
| receptor | | | | | |
| Glial fibrillary | Rabbit | 1:80 | | | Sigma |
| acidic protein | | | | | |
| (GFAP) | | | | | |
| Glial fibrillary | Rabbit | | | 1:50 | Dako |
| acidic protein | | | | | |
| (GFAP) | | | | | |
| Human nestin | Rabbit | 1:200 | 1:2500 | | Kindly |
| | | | | | supplied |
| | | | | | by Messam |
| | | | | | CA., NIH |
| Microtubules | Mouse | 1:50 | | | Zymed |
| associated | | | | | |
| protein 2 | | | | | |
| (MAP2) | | | | | |
| Neurofilament | Mouse | 1:200 | 1:1000 | 1:50 | Sigma |
| 200 (NF-H) | | | | | |
| Neuron specific | Mouse | 1:100 | 1:1000 | 1:50 | Cymbus |
| enolase (NSE) | | | | | Biotechnology |
| Neuronal | Mouse | 1:50 | 1:2500 | | Chemicon |
| nuclei (NeuN) | | | | | International |
| Tryptophan | Mouse | | 1:200 | | Sigma |
| hydroxylase | | | | | |
| (TPH) | | | | | |
| Tyrosine | Rabbit | 1:1000 | 1:6000 | | Chemicon |
| hydroxylase | | | | | International |
| (TH) | | | | | |
| Tyrosine | Rabbit | | | 1:50 | Calbiochem |
| hydroxylase | | | | | |
| (TH) | | | | | |
| Vesicular | Rabbit | 1:50 | | | Chemicon |
| monoamine | | | | | International |
| transporter 2 | | | | | |
| (VMAT2) | | | | | |

*IA = immunoassay; WB = Western blot; FC = Flow cytometer

TABLE 4
Anti-mouse, anti-rabbit and anti-human secondary antibodies

| | | Dilution | Dilution for | Dilution | | |
|---|---|---|---|---|---|---|
| Specificity | Source | for IA* | WB* | for FC* | Conjugated | Source |
| Mouse | Goat | 1:100 | | 1:100 | CyTM2 | Jackson |
| | | | | | | ImmunoResearch |
| | | | | | | Laboratories |
| Mouse | Donkey | 1:300 | | | CyTM3 | Jackson |
| | | | | | | ImmunoResearch |
| | | | | | | Laboratories |
| Mouse | Sheep | 1:50 | | | FITC | Sigma |
| Mouse | Goat | | 1:20000 | | Peroxidase | Jackson |
| | | | | | | ImmunoResearch |
| | | | | | | Laboratories |
| Rabbit | Donkey | 1:300 | | | Cy ™2 | Jackson |
| | | | | | | ImmunoResearch |
| | | | | | | Laboratories |
| Rabbit | Goat | 1:400 | | 1:100 | Cy ™3 | Jackson |
| | | | | | | ImmunoResearch |
| | | | | | | Laboratories |
| Rabbit | Swine | 1:20 | | | FITC | Dako |
| Rabbit | Goat | | 1:25000 | | Peroxidase | Jackson |
| | | | | | | ImmunoResearch |
| | | | | | | Laboratories |

*IA = immunoassay; WB = Western blot; FC = Flow cytometer

Results:

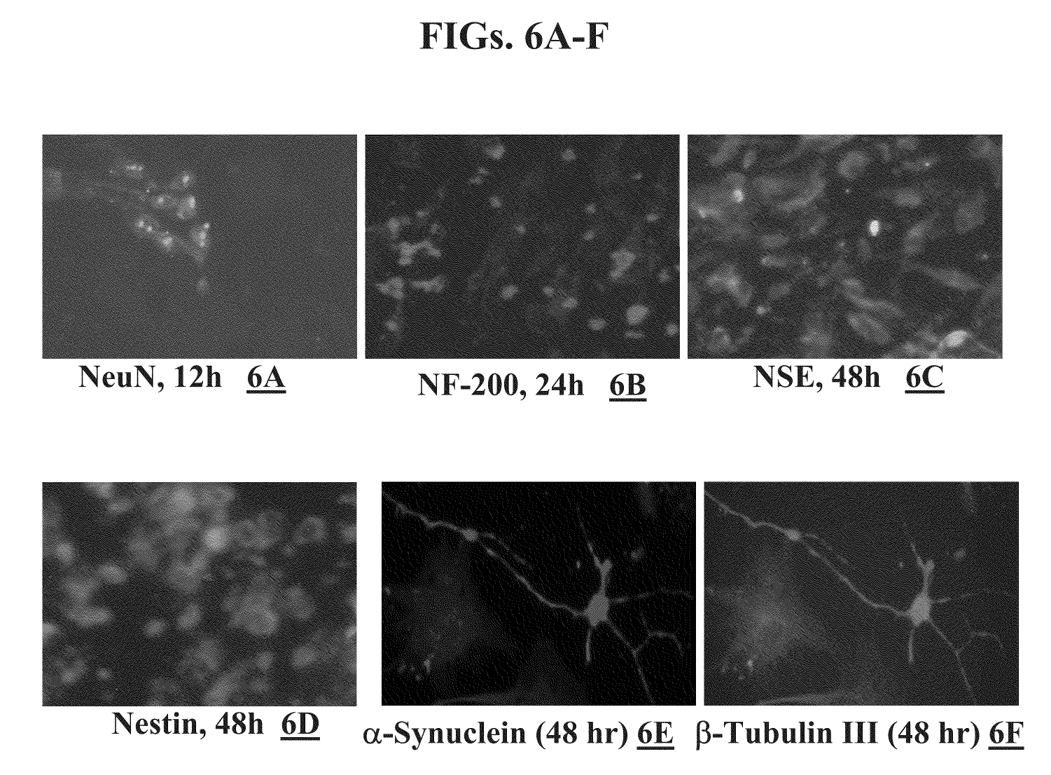

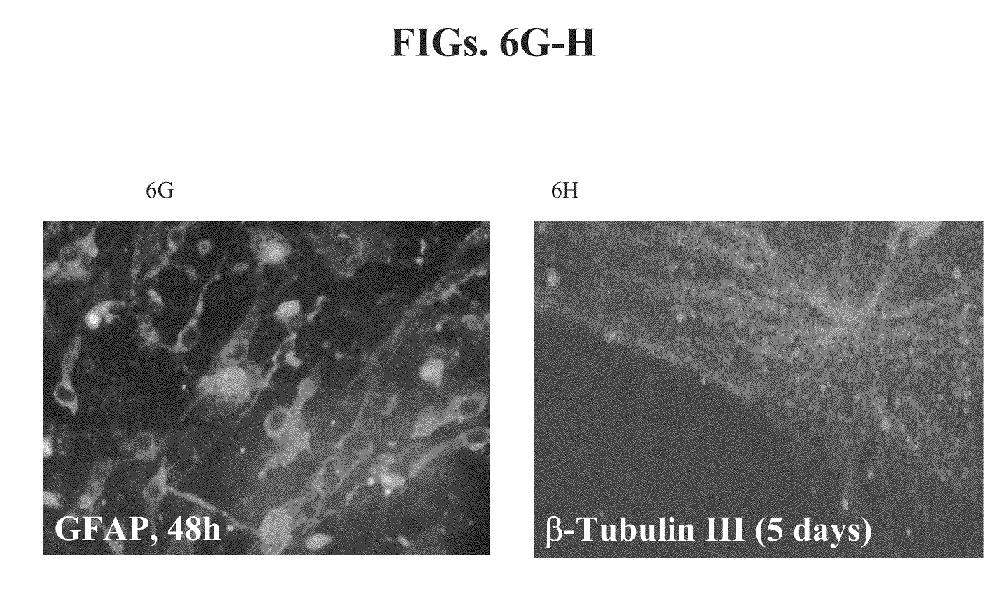

Immuno-staining revealed the presence of neuronal markers NeuN, NF-200, NSE, nestin, α-synuclein and β tubulin in differentiating hBMSc 12, 24, 48 and 48 hr following differentiation induction, respectively (FIGS. 6A-6F respectively). Antibody-labeled GFAP and β-tubulin III were observed in hBMSc 48 hr and 5 days following differentiation induction, respectively (FIGS. 6G-H).

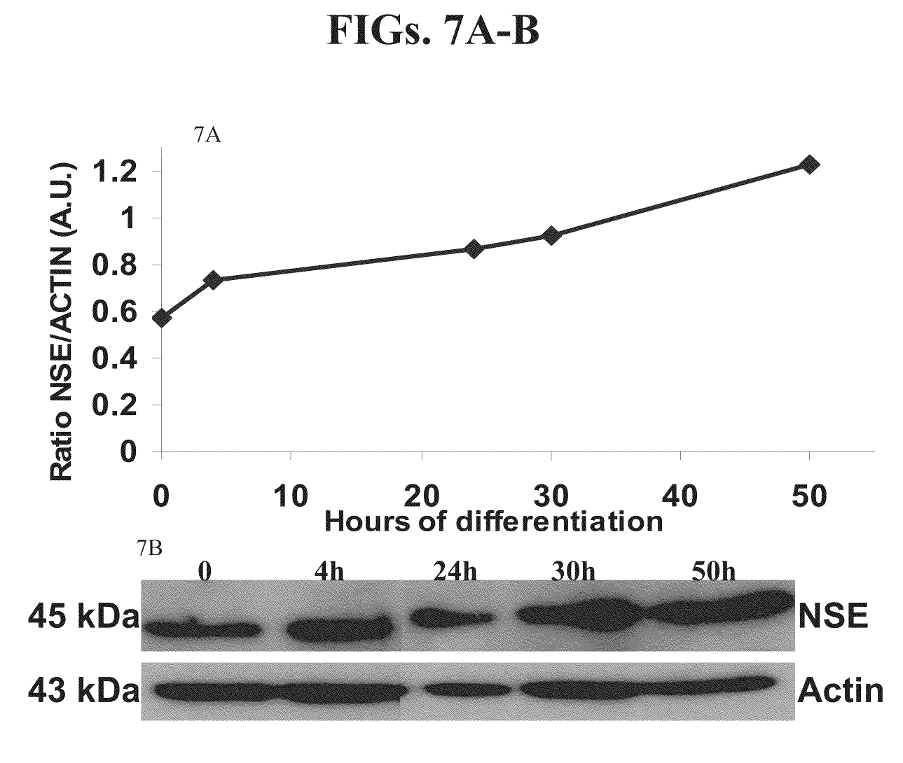

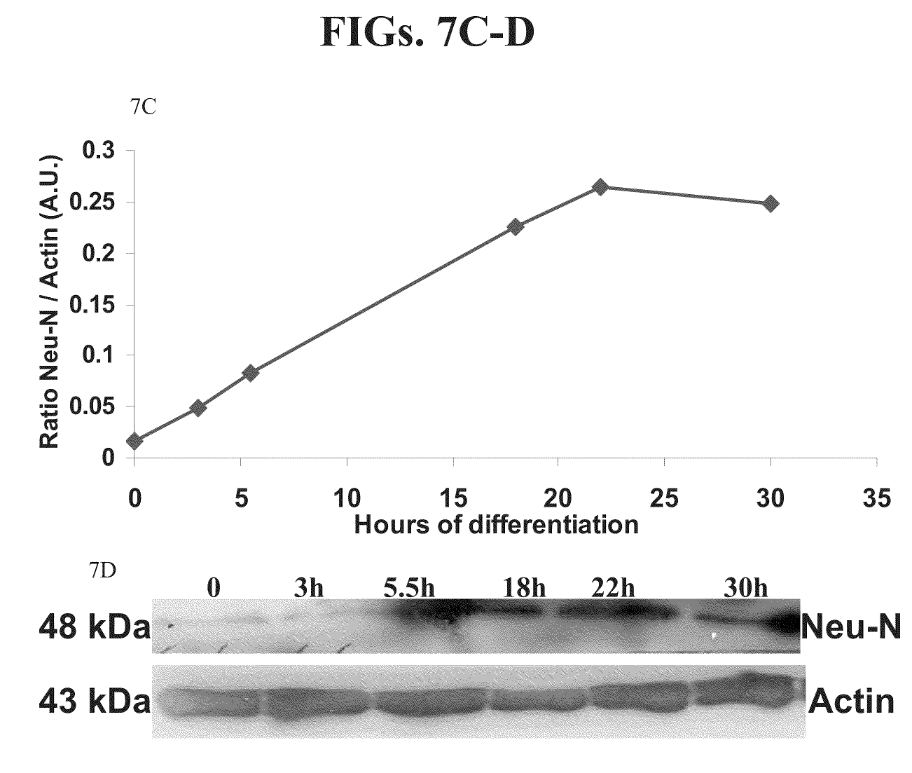

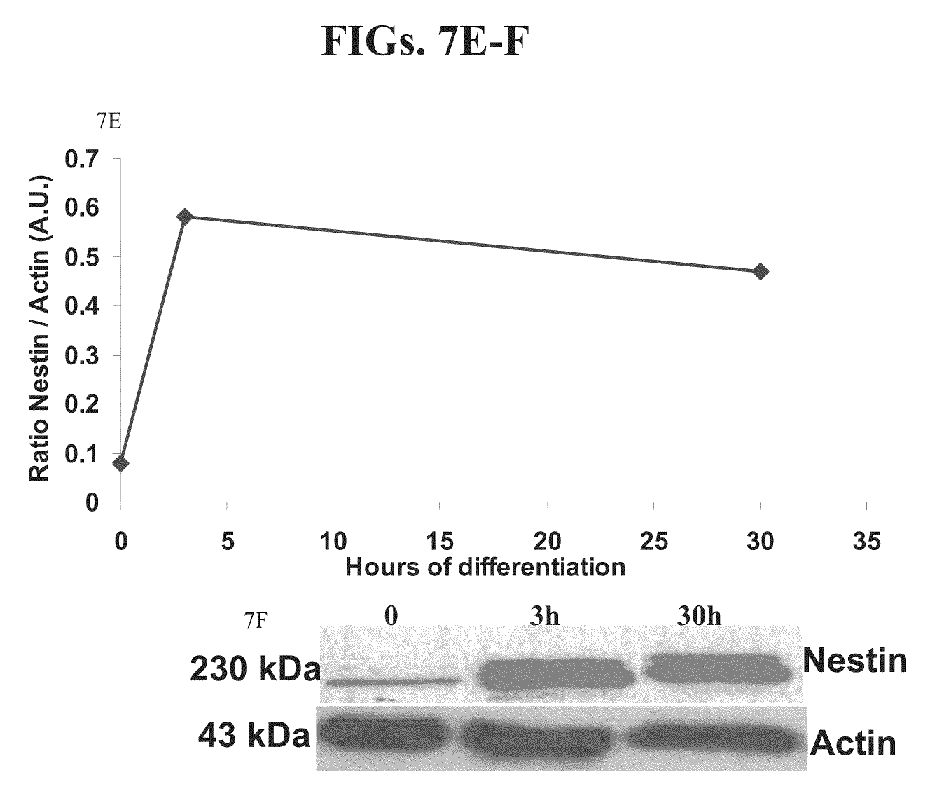

The expression of Neu-N, (FIGS. 7A-7B) NSE (FIGS. 7C-7D) and nestin (FIGS. 7E-7F) proteins (relative to actin), increased substantially following differentiation induction, as indicated by Western blot analysis.

### Example 5

### Long-Term Survival of Differentiating hBMSc

Methods:

Long-Term Differentiation Culture:

hBMSCs were incubated in the “differentiation medium I” (see Example 2 above) for 24 hr at 37° C. then transferred to a “differentiation medium II” for an additional 48 hr followed by “long-term differentiation medium” for “long-term differentiation medium” for 28 days at 37° C.

TABLE 5
Long-term differentiation medium

| | |
|---|---|
| Long Neuronal | DMEM or DMEM/F12; 2 mM L-glutamine; SPN; |
| differentiation | *N-2 or B27 supplement; 10 ng/ml bFGF; 10 ng/ml |
| medium (4 weeks) | human glial cell line-derived neurothrophic |
| | factor (GDNF); 10 ng/ml human β-nerve growth |
| | factor ((βNGF); 1 mM dbcAMP; 0.5 mM IBMX; |
| | 10 μM DHA |

Immunoassay:

Cells were plated and treated in slide chambers (Nalge Nunc International) previously treated aseptically with poly-L-lysine (Sigma). The cells were fixed with 4% paraformaldehyde in PBS (pH 7.3) for 30 min at 4° C. and 30 min a room temperature. The slides were then washed three times with PBS (5 min each) and permeabilized with PBS containing 0.1% Triton X-100 (Sigma) and 10% goat serum (Biological Industries) for 10 min at 4° C. and 10 min at room temperature. The slides were then washed three times with PBS (5 min each). The endogenous peroxide was blocked by adding 3% H₂O₂ (Merck) in methanol absolute (Bio-Lab, Israel) for 20 min at room temperature. Following three washes in PBS (5 min each), slides chambers were incubated overnight at 4° C. with anti-MAP2, anti-β-tubulin III, or anti-nestin diluted as described in Tables 3-4 in Example 4 hereinabove. On the next day, the slides were washed thoroughly three times in PBS (10 min each) then incubated for 30 minutes at room temperature with Cy™3-conjugated goat anti-rabbit IgG or Cy™2-conjugated goat anti-mouse IgG (Jackson ImmunoResearch Laboratories) in 10% goat serum and 0.2% Tween-20 in PBS. Following incubation, the slides were washed three times with PBS (5 min each), mounted with glycerol vinyl alcohol mounting solution (Zymed Laboratories), covered with glass slips and examined under a florescence microscope.

Results:

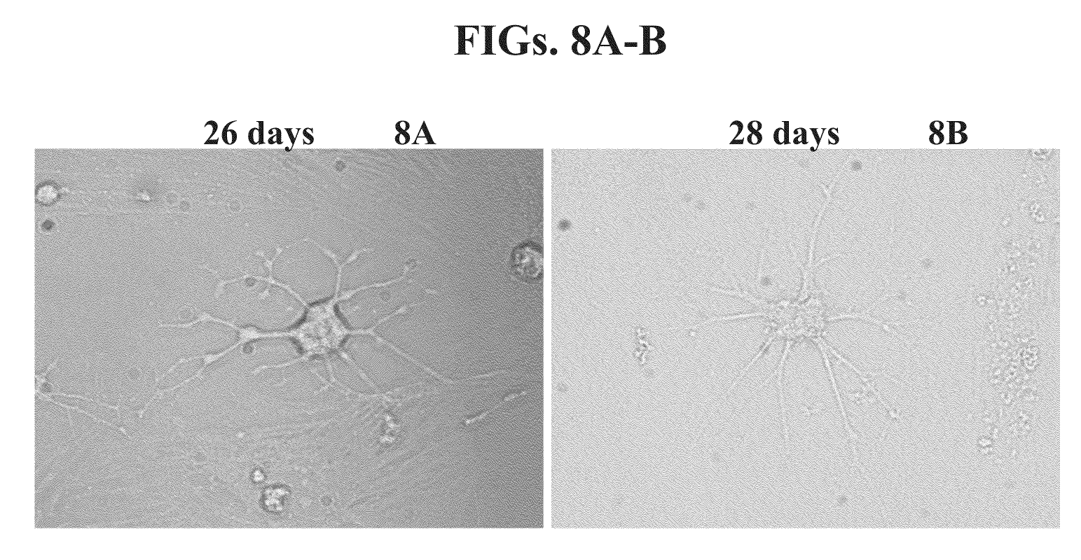

hBMSc exhibited typical neuron-like cell morphology following 28 day incubation in the “long-term differentiation medium” (FIGS. 8A-B).

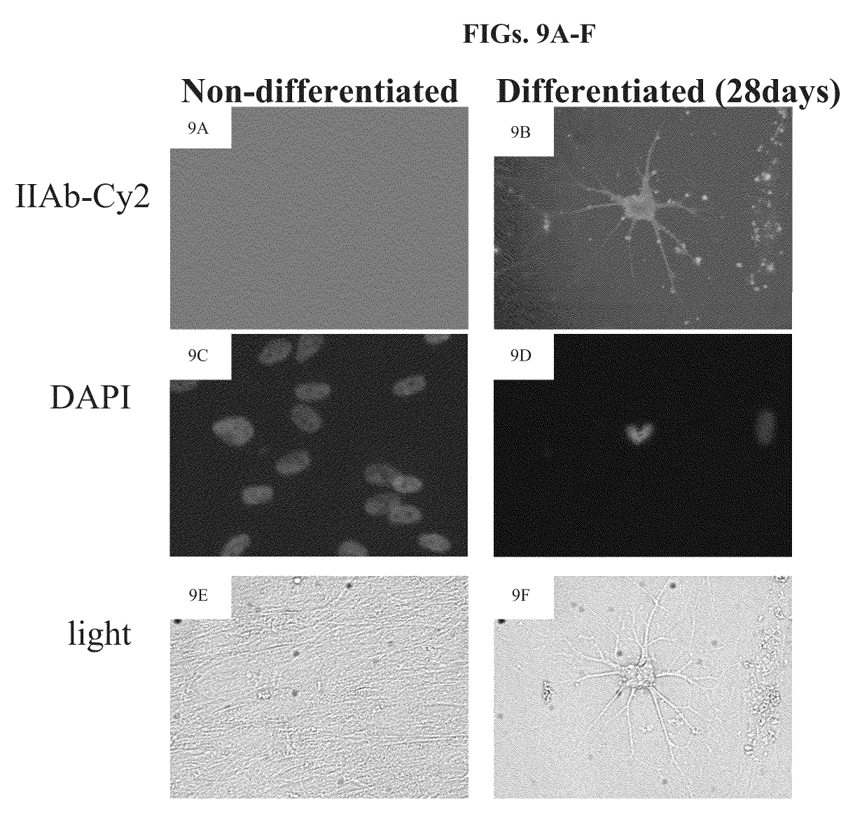

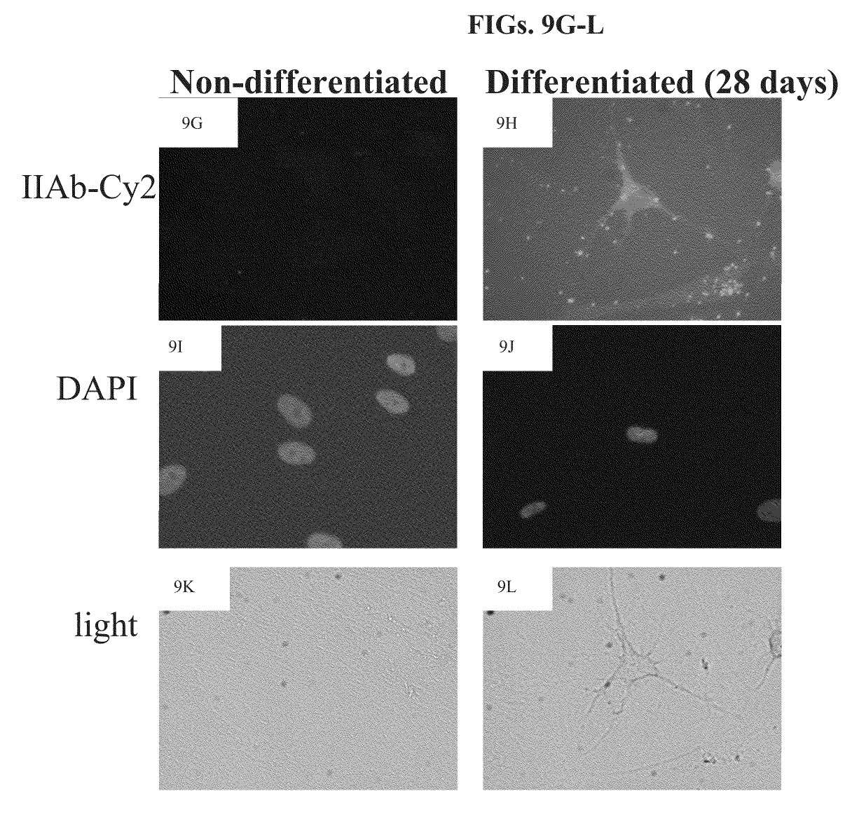

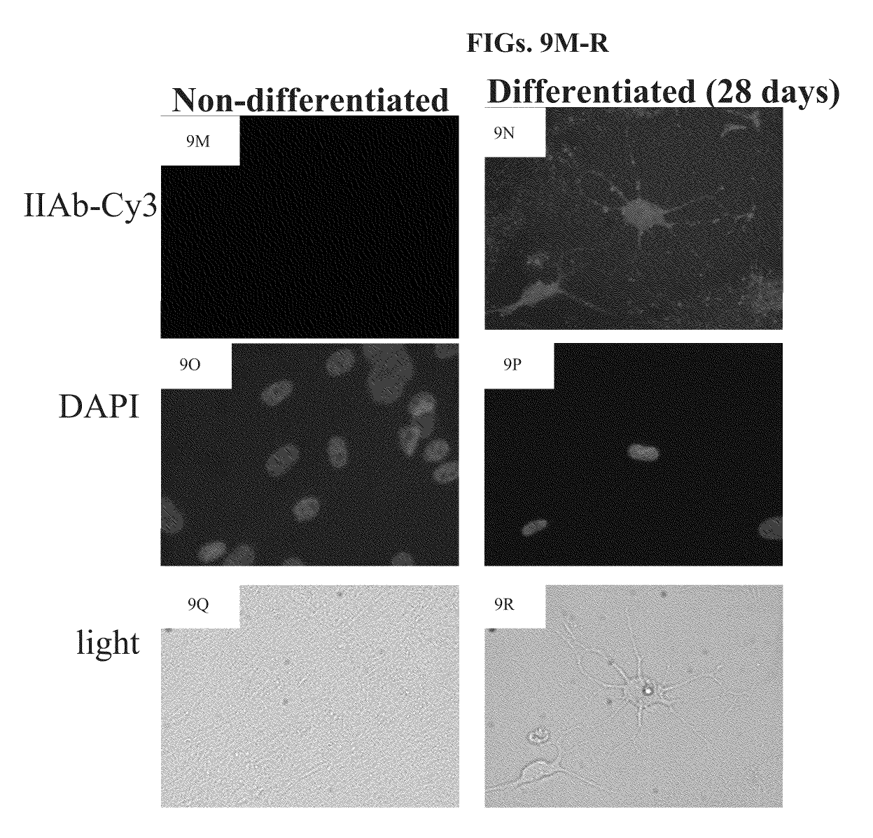

Antibody-labeled neuronal markers MAP2 (microtubule-associated protein 2), (FIGS. 9A-F), β-tubulin III (FIGS. 9G-L, and nestin (FIGS. 9M-9R, were observed in hBMSc following 28 day incubation.

### Example 6

### Expression of Dopaminergic mRNAs in Differentiated hBMSc

Methods:

RT-PCR:

hBMSc were cultured for 12-72 hr in the “proliferation medium” and in the “differentiation medium” (see Example 1-2 hereinabove). Total RNA was extracted from the hBMSc by using the guanidine isothiocyanate method as described by Chomczynski & Sacchi (1987). The RT-PCR procedure designed for identifying dopaminergic markers was performed essentially as described in Example 3 hereinabove except for using the primers of SEQ ID NOs: 3-8, 13-14 and 27-28.

### Results:

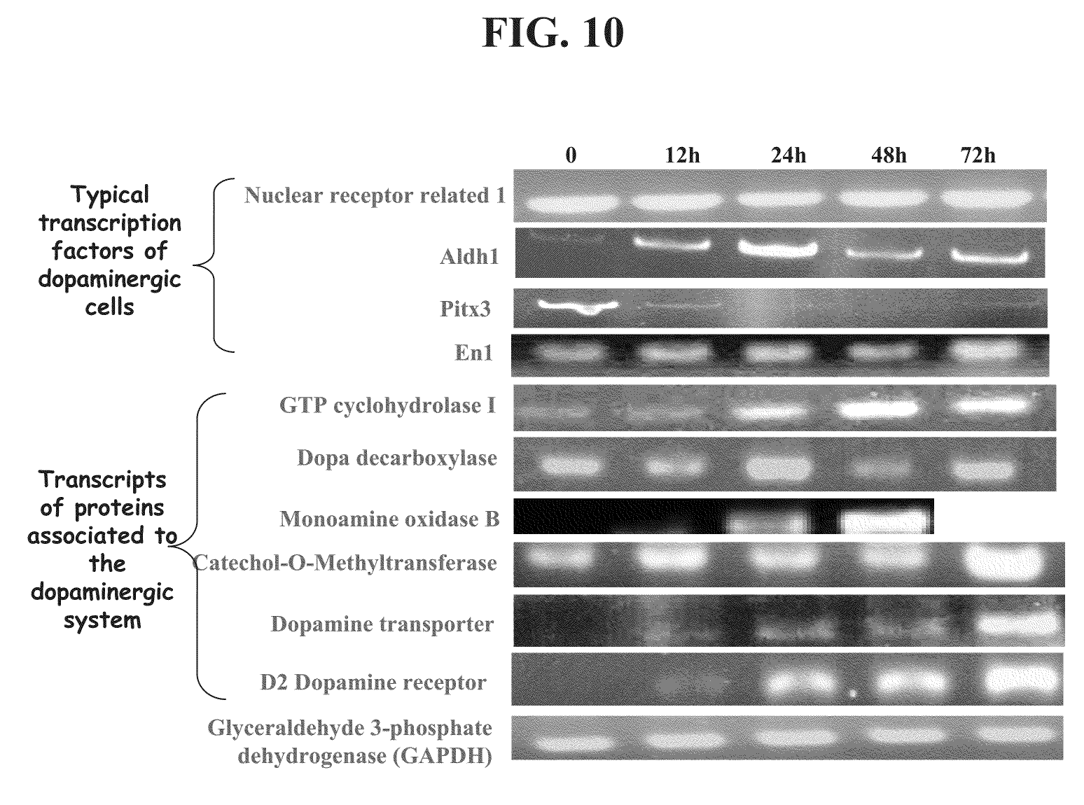

As can be seen in FIG. 10, transcripts of several dopaminergic markers were expressed in differentiated and/or non-differentiated hBMSc. These include Nurr1 [nuclear receptor related 1] and En-1 [engrailed 1]; transcription factors that play roles in the differentiation of midbrain precursors into dopamine neurons], Aldh1 [aldehyde dehydrogenase 1], and AADC [aromatic L-amino acid decarboxylase; the enzyme which catalyzes the decarboxylation of L-3,4-dihydroxyphenylalanine (L-DOPA) to dopamine, L-5-hydroxytryptophan to serotonin and L-tryptophan to tryptamine]. Transcriptional expression of GTP cyclohydrolase 1 [the enzyme necessary for production of tetrahydrobiopterin (BH4) cofactor for TH] and MAO-B [monoamine oxidase B; involve in the breakdown of dopamine] were markedly higher in the differentiated hBMSc, as compared with the non-differentiated hBMSc, while transcripts of D2 dopamine receptor, COMT [catechol-o-methyltransferase; involve in the breakdown of dopamine] and DAT dopamine transporter were expressed only in the differentiated hBMSc.

### Example 7

### Induction of Tyrosine Hydroxylase in Differentiated hBMSc

Methods:

Real Time PCR:

Total RNA was extracted from hBMSc using the guanidine isothiocyanate method as described by Chomczynski & Sacchi (1987). cDNA was generated as described in Example 3 hereinabove by carrying out a RT reaction with random primers. Amplification of cDNA was performed in an ABI Prism 7700 sequence detection system (Applied Biosystems) using TaqMan universal PCR master mix using specific primers of human TH and 18S rRNA (Applied Biosystems). Stimulated conditions for sample normalization were applied by amplification of 18S rRNA. The amplification protocol was 80 cycles of 95° C. for 20 and 61° C. for 1 min. Quantification of gene expression relative to 18S rRNA was calculated by the protocol's ΔΔCT method and from standard curve method.

Western Blot Assay:

The assay was performed as described in Example 4 hereinabove except for immunoblotting with anti-TH and anti-actin antibodies.

Immunoassay:

The assay was performed as described in Example 5 hereinabove using anti-TH antibody as described in Tables 3-4 in Example 4 hereinabove.

Results:

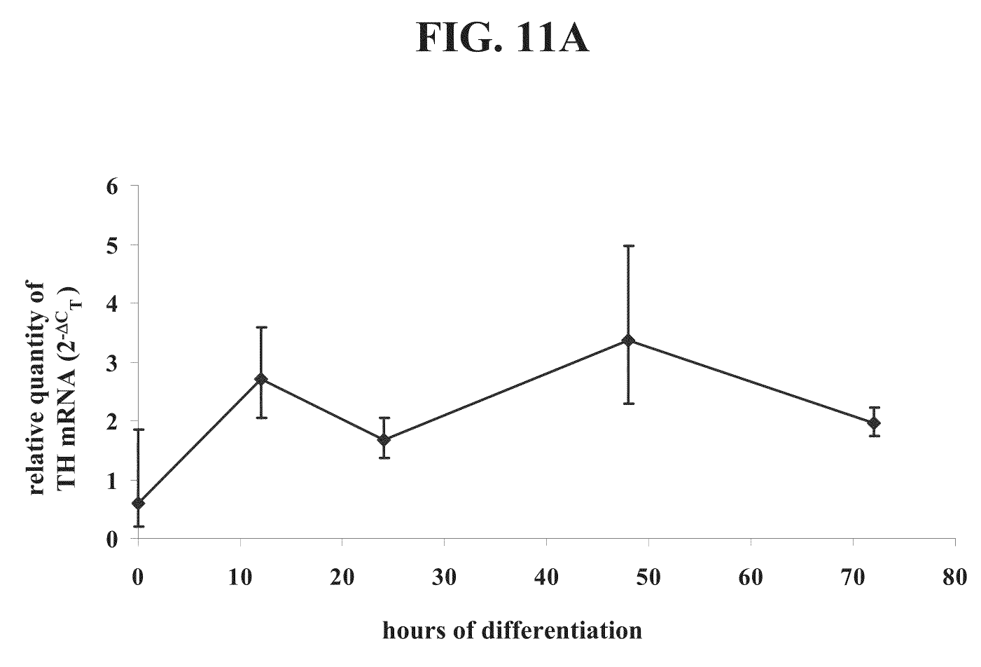

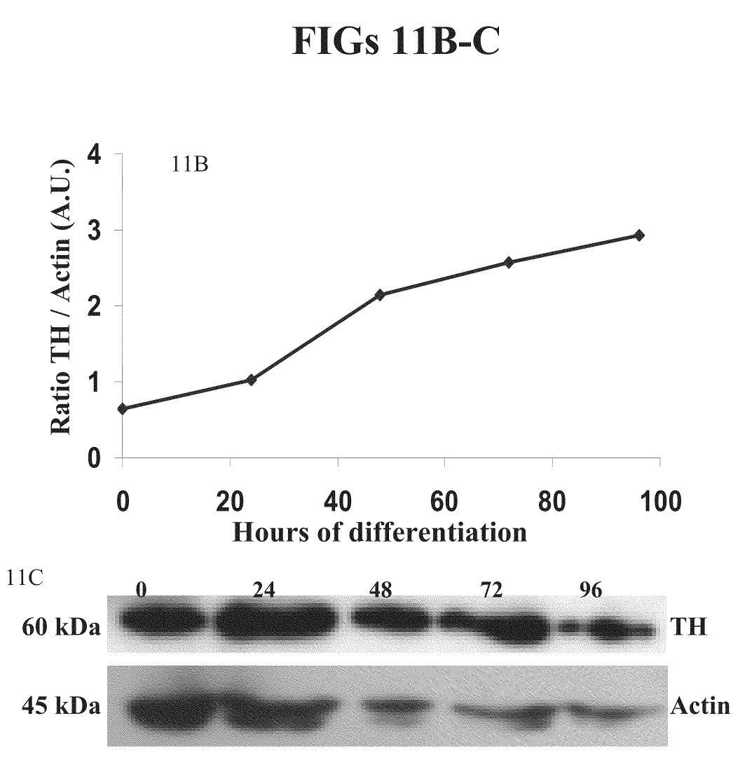

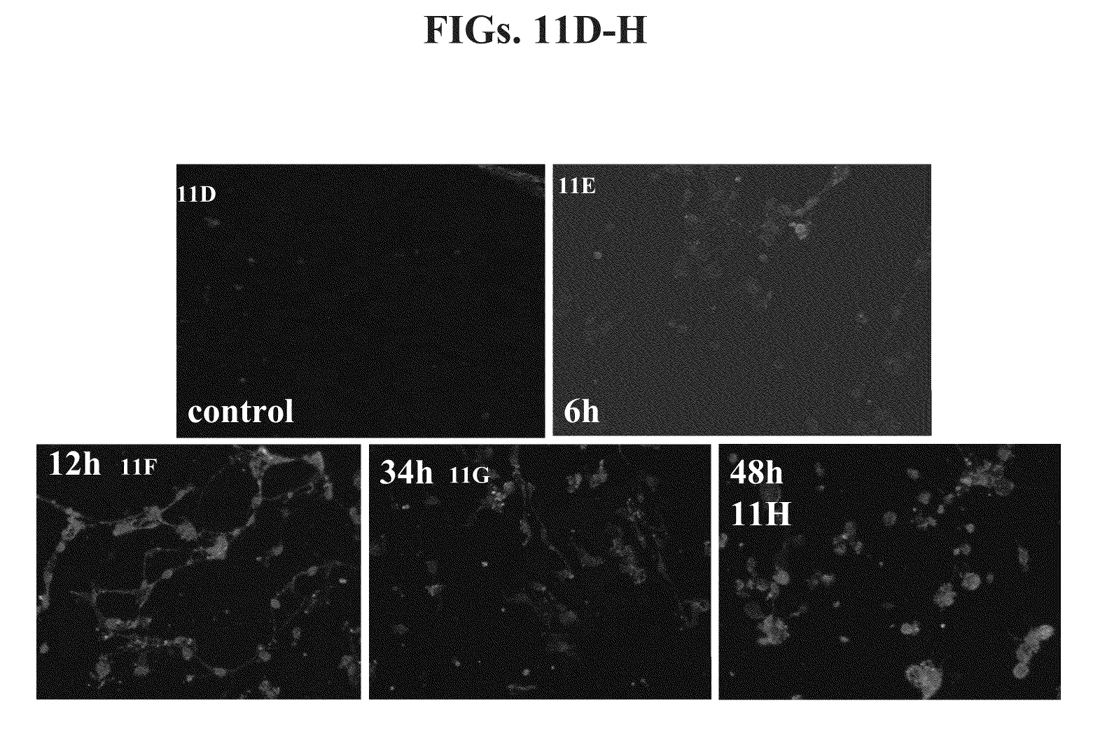

Tyrosine hydroxylase mRNA (FIG. 11A) and protein levels (FIG. 11B-11C) were substantially elevated during neuronal differentiation of hBMSc. In addition, the presence of antibody-labeled tyrosine hydroxylase was observed in differentiating hBMSc, 6 to 48 hours following differentiation induction (FIGS. 11D-H).

### Example 8

### Identification of Dopamine-Related Proteins in hBMSc

Methods:

Immunoassay:

hBMSc were incubated for five days in the “differentiation medium” (see Example 2 hereinabove), then harvested and stained with a specific antibody against vesicular monoamine transporter 2 (VMAT-2) using the procedure described in Example 5 above. Antibody binding to VMAT-2 in cells was visualized by using a secondary Cy™3-conjugated antibody. The stained cells were observed under a laser confocal microscope LSM 510 (ZEIZZ, Germany).

Flow Cytometry:

Following 48 hours incubation in the “differentiation medium” (see Example 2 hereinabove), the cells were harvested and suspended in 0.05% trypsin and 25 mM EDTA in phosphate-buffered saline (PBS). Cell suspensions (0.5×10⁶ cells/ml), were incubated for 30 minutes with antibodies specific against D2 dopamine receptor for as described in Tables 3-4 in Example 4 above. The antibody-labeled cells were thoroughly washed with two volumes of PBS and fixed in flow buffer (1% paraformaldehyde, 0.1% sodium azide, and 0.5% bovine serum albumin in PBS). The washed cells were analyzed by a FACSCalibur™ flow cytometer (Becton Dickinson).

Results:

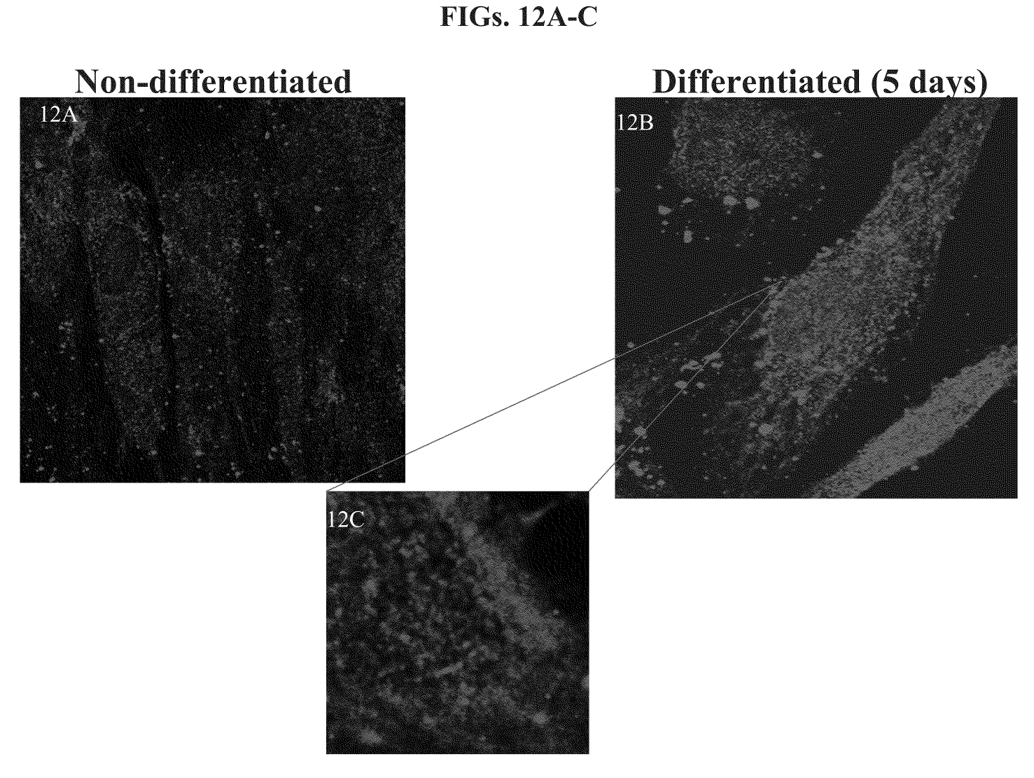

Confocal fluorescent microscope images of hBMSc revealed that antibody-labeled VMAT2 domaminogenic marker was present in differentiated hBMSc but not in the non-differentiated cells (FIGS. 12A-C-B).

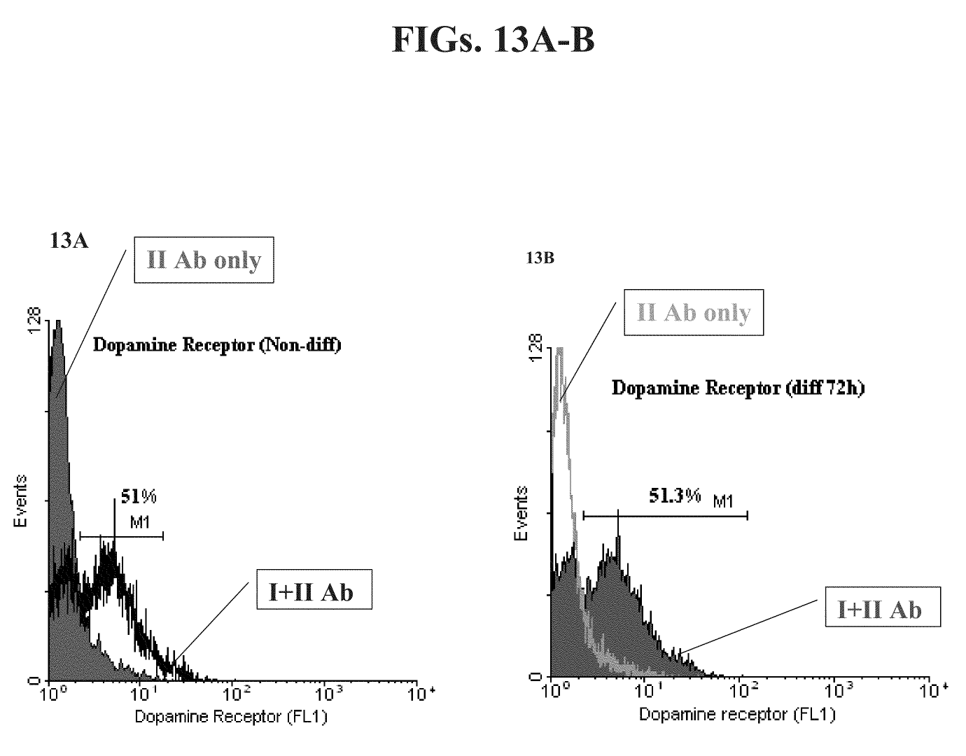

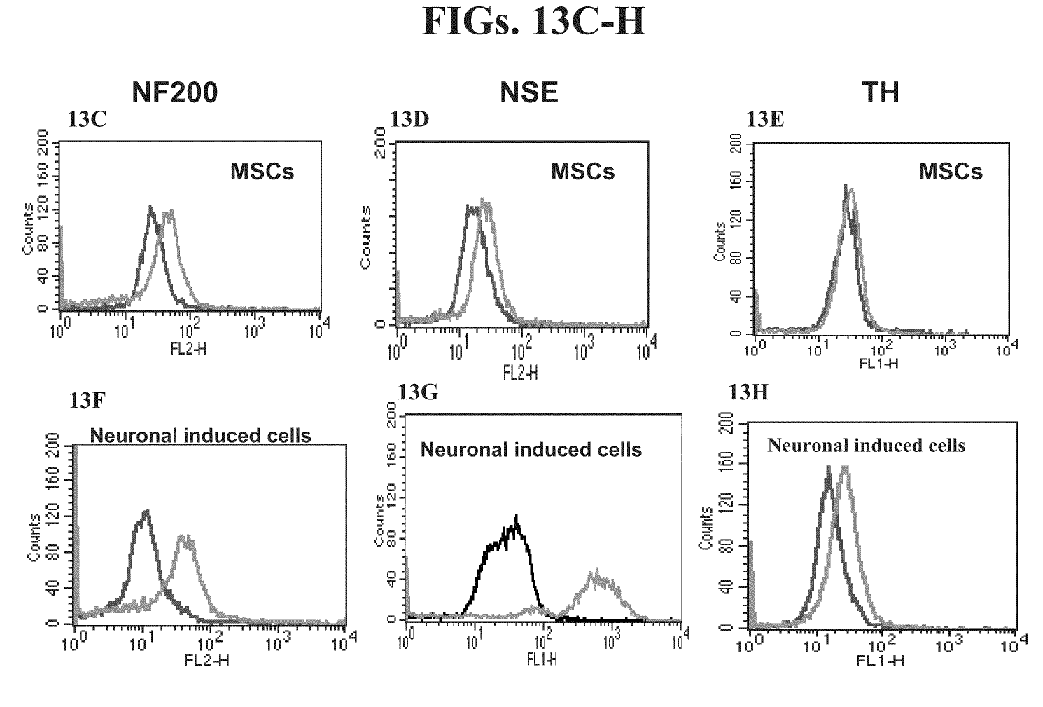

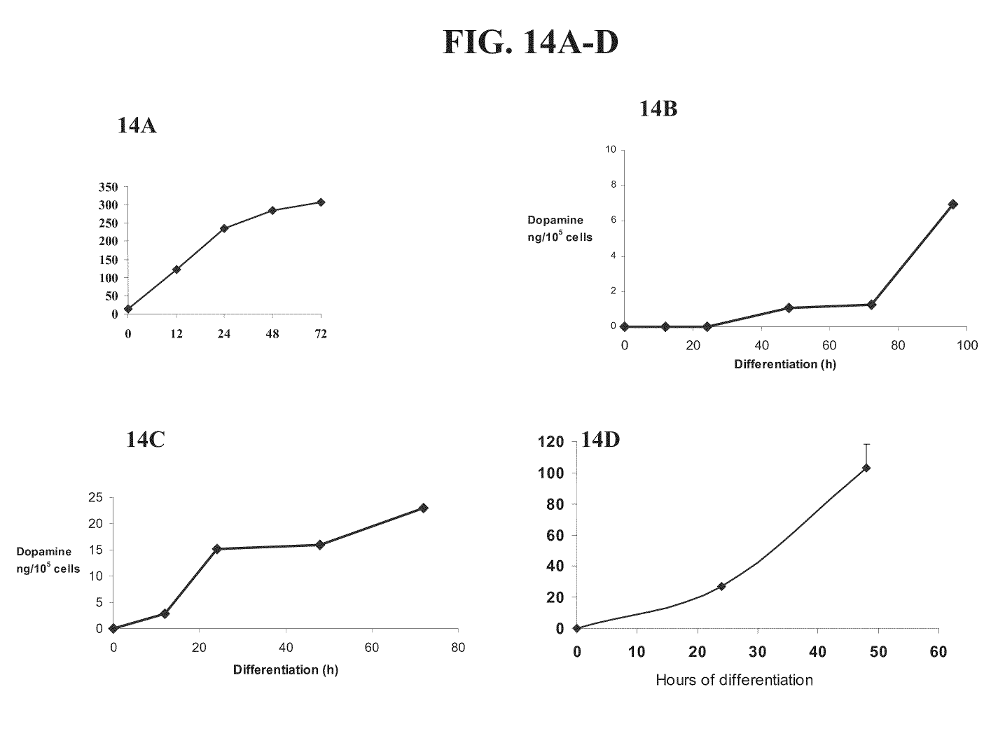

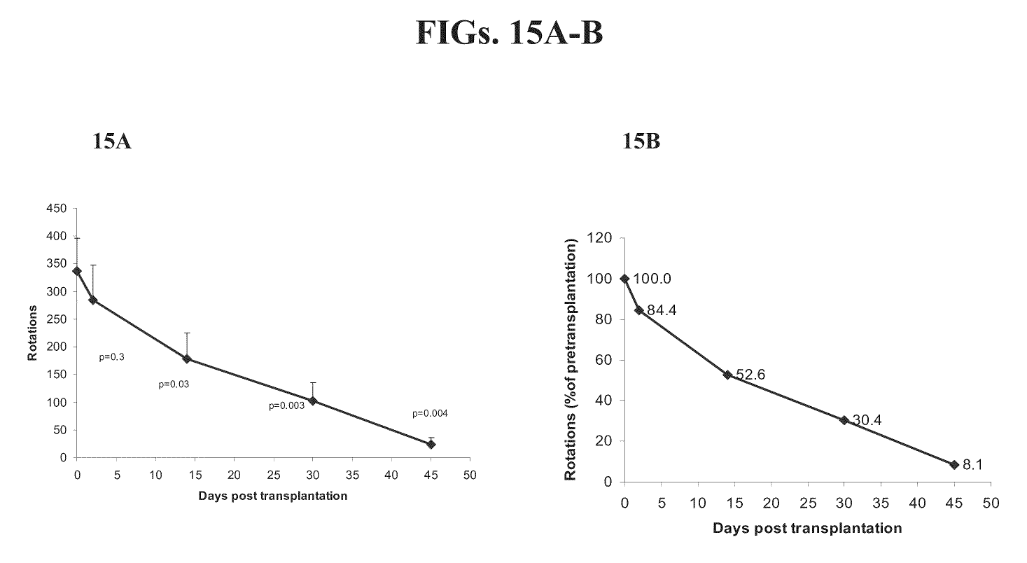

Similarly, flow cytometer analysis shows that the dopaminogenin marker D2 expressed in differentiated hBMSc but not in the non-differentiated cells (FIG. 13A). NSE (FIGS. 13D and 13G), NF-200 (FIGS. 13C and 13F) and TH (FIGS. 13E and 13H) were present in the differentiated hBMSc.

### Example 9

### Dopamine Secretion by Differentiating hBMSc is Induced by Neurotrophic Factors

Methods:

Cell Culture:

hBMSc were cultured as described in Table 6 below.

TABLE 6
Culture media used to induce dopamine secretion by hBMSc

| | |
|---|---|
| Stage 1: | Dulbecco's modified eagle medium (DMEM; without HEPES); 100 μg/ml |
| Proliferation medium | streptomycin, 100 U/ml penicillin, 12.5 units/ml nystatin |
| (weeks) | (SPN); 2 mM L-glutamine; 5% horse serum; 15% fetal calf serum; |
| | 0.001% 2-β-mercaptoethanol; Non-essential amino acids X1; 10 ng/ml |
| | human epidermal growth factor (EGF) |
| Stage 2: | DMEM/F12 (without HEPES); 2 mM L-glutamine; SPN; 10 ng/ml |
| Additional | human basic fibroblast growth factor (bFGF);; 10 ng/ml EGF; *N2 |
| differentiation | supplement; 40 μM arachidonic acid; 10-40 μM docosahexaenoic |
| medium | acid (DHA); 40 μM Vit-E; 10 ng/ml fibroblast growth factor 8 |
| (24-72 hr) | (FGF8); 200 ng/ml sonic hedgehog (Shh) |
| Stage 3: | DMEM/F12; 2 mM L-glutamine; SPN; *N2 supplement; 200 μM |
| Dopaminergic | ascorbic acid; 1 mM dibutyryl cyclic AMP; 0.5 mM |
| differentiation | isobutylmethlxanthine; 1 μM retinoic acid; 200 μM butylated |
| medium | hydroxyanisole (BHA);; human transforming growth factor β3 |
| (12-96 hr) | (TGF-β3), 2 ng/ml; human galia-derived neurotrophic factor: |
| | (GDNF), 2 ng/ml; human neurturin: (hNTN), 20 ng/ml; human |
| | brain-derived neurotrophic factor: (BDNF), (10 ng/ml; human |
| | neurotrophin: (hNT-3), 20 ng/ml; human interleukin-1β (hIL-1β), |
| | 100 pg/ml; |

*N2 supplement: insulin 25 ng/ml; progesterone 20 nM; putrescin 100 μM; selenium 30 nM; transferrin 100 μg/ml.

HPLC Analysis:

Samples were stabilized by adding 88 μl of 85% orthophosphoric acid and 4.4 mg of metabisulfite to ml sample. Dopamine was extracted by aluminium adsorption (Alumina, Bioanalytical Systems Inc.). Separation of injected samples (50 μl) was effected by isocratic elution on a HPLC-electron chemical detection (HPLC-ECD) system with a reverse-phase C18 column (125×4.6 mm dimension, Hichrom, Inc.) in a monochloroacetate buffer mobile phase. The flow rate was set at 1.2 ml per min, and the oxidative potential of the analytical cell was set at +650 mV. Results were validated by co-elution with dopamine standards under varying buffer conditions and detector settings.

Results:

The amount of dopamine measured in the supernatant of differentiating hBMSc increased from a non-detectable level to about 23 ng/ml (10⁵ cells) during the 72 hours incubation period in the “dopaminergic differentiating medium” (FIG. 14A). Inducing cell polarization by KCl (supplementing the medium with 56 mM KCl followed by 10 minutes incubation) further enhanced dopamine secretion (FIG. 14B). The amount of DOPA (dopamine precursor) synthesized by the differentiating hBMSc increased from about 10 to 300 pg/ml (10⁵ cells) during the 72 hours incubation period in the “dopaminergic differentiating medium” (FIG. 14C), while the amount of DOPAC (dopamine metabolite) increased from a non-detectable level to about 105 ng/ml (10⁵ cells) during the 50 hours incubation period in the “dopaminergic differentiating medium” (FIG. 14D).

### Example 10

### Transplantation of Mouse BMSc in the Striatum of a Rat Model for Parkinson's Disease Improves Rotational Behavior

Methods:

Generating Mouse Bone Marrow Stromal Cells (Mouse BMSc):

Mouse BMS cells were obtained from transgenic male mice bearing the enhanced green fluorescent protein (Tg-EGFP; Hadjantonakis et al., 1998). The mice were sacrificed by cervical dislocation and the tibias and femurs were removed and placed in Hank's balanced salt solution (HBSS). Mouse bone marrow cells were collected by flushing out the marrow using a syringe (1 ml) with 25G needle, filled with 0.5 ml sterile HBSS. The collected cells were disaggregated by gentle repeat pipetting until a milky homogenous single-cell suspension was achieved. The single-cell suspension was washed in 5 ml HBSS and centrifuged under 1000 g for 20 min at room temperature. Following centrifugation, the supernatant was discarded and the cell pellet was resuspended in 10 ml growth medium.

Cell Culture:

Isolated mouse bone marrow cells were cultured in the “proliferation medium” (see in Example 1 hereinabove) and incubated for 48 hr at 37° C. The non-adherent layer was then discarded and the tightly adhered cells were washed twice with PBS and cultured in a fresh “proliferation medium”. The growth medium was replaced every 3-4 days until cells reached 70%-90% confluency. The cells were then harvested and mixed in a trypsin-EDTA solution (0.05% trypsin and 25 mM EDTA in PBS), incubated for 5 minutes at 37° C., then transferred to the “additionaldifferentiation medium” (see in Example 2 hereinabove) for an additional incubation of 72 hr at 37° C. The differentiated cells were washed in PBS and induced for neural-like differentiation by incubation in the “differentiation medium” (see in Example 1 hereinabove) for 12-72 hr at 37° C.

Mouse BMSc Transplantation:

neural-differentiated bone marrow stromal cells (mBMSc) were injected in the substania nigra of female 6-OHDA lesioned rats using stereotactic frame (as described by Bjorklund et al., 2002). Saline injection was used as a control.

Rotational Behavior Analysis:

6-OHDA-lesioned rats were treated with amphetamine 5 mg/kg to induce rotational behavior. The rotational response to amphetamine was examined 3, 15, 30 and 45 days post transplantation using a computerized rotameter (San Diego Instruments).

Results:

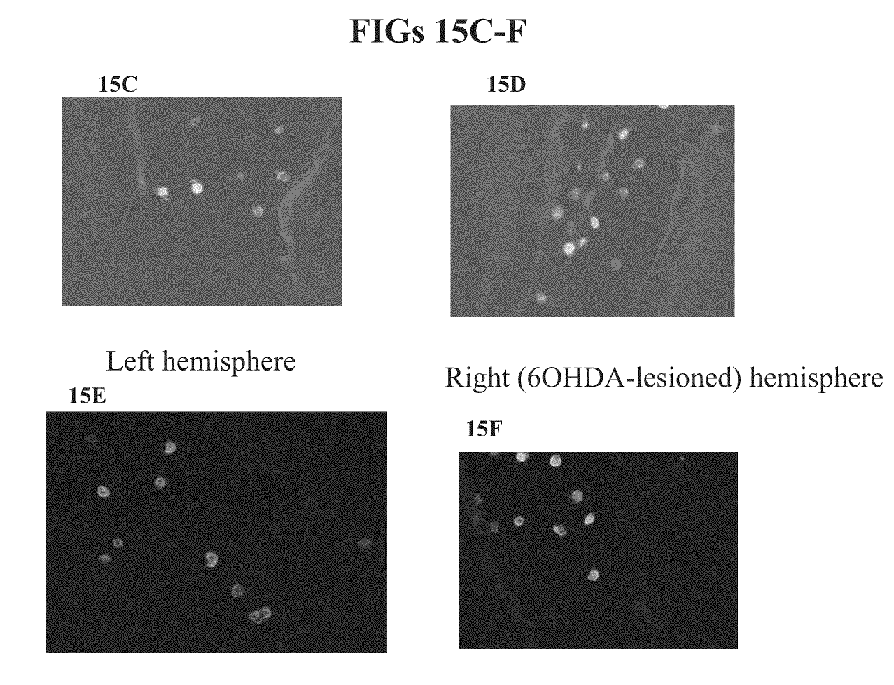

Transplantation of neuronal-induced mBMSc into amphetamine-induced 6-OHDA rats substantially reduced rotational behavior of the rats, from about 340 to just 25 rotations per 2 hr, 45 days post transplantation (FIG. 15A). The relative rotation rate of the treated rats was reduced by 97.9%, as compared with saline-treated rats, 45 days post transplantation (FIG. 15B).

### Example 11

### Survival and Migration of Transplanted Mouse BMSc in Rat Brain

Methods:

Neuronal differentiated Tg-EGEF mouse BMSc were prepared and transplanted in the substania nigra of 6-OHDA rats (both hemispheres), as described in Example 10 hereinabove. Treated and untreated (saline only) rats were sacrificed 45 days post transplantation. Tissue sampled from lesioned and non-lesioned rat hemispheres were sectioned and observed under a fluorescent microscope, for the presence of green fluorescent protein (GFP) marking the transplanted mBMSc.

Results:

mBMSc survived in the treated rats substania nigra (FIG. 15C-D) and immigrated into the treated rats striatum (FIG. 15E-F), 45 days after the nigral transplantation. In addition, transplanted cells successfully migrated to the cortex and striatum (FIGS. 15C-F). In rats that were injected with saline, the rotational behavior did not change.

### Example 12

### Mouse BMSc Differentiated into Oligodendrocytes Precursors

Methods:

Cell Culture:

Mice B5/EGFP (male) were sacrificed by cervical dislocation and were prepared with 70% alcohol solution. After tibias and femurs were removed and placed in Hank's balanced salt solution (HBSS; Biological Industries, Bet-Haemek, Israel), mouse bone marrow cells were collected by flushing out the marrow using a syringe (1 ml) with 25G needle, filled with 0.5 ml sterile HBSS. Cells were disaggregated by gentle pipetting several times until a milky homogenous single-cell suspension was achieved. Bone marrow aspirate was diluted and washed by adding 5 ml HBSS, centrifuged at 1000 g for 20 min at room temperature (RT), and removing supernatant. The cell pellet was resuspended in 1 ml growth medium and diluted to 10 ml. The cells were plated in polystyrene plastic tissue cultures 75 cm² flask (Corning Incorporated, Corning, N.Y.) in the “proliferation medium” (see Example 1 hereinabove) for one week. The cells were then transferred to polylysin-coated slide-chambers (3200 cells/well), supplemented “proliferation medium” and incubated for 24 hours at 37° C. The growth medium was then replaced with “oligodendrocytes differentiation media” composed of DMEM supplemented with 2 mM glutamine, SPN, one or more of the following substances: bFGF (10 ng/ml), EGF (10-20 ng/ml), Interlukin-1b (20-40 ng/ml), dbcAMP (1-2 mM), retinoic acid (0.5 or 1 μM), neurotrophin-3 (50 or 100 ng/ml), human platelet derived growth factor (PDGF-AA; 5-20 ng/ml), N2 supplement, triiodothyronien (T3; 40 ng/ml) and ciliary neurotrophic factor (20 ng/ml; CNTF).

Immunoassay:

The cultures were incubated at 37° C. for 1, 2 or 6 days (replacing growth media with fresh media every two days) then fixed in 4% PFA. Cells were blocked in 10% FCS solution then incubated with 5 ug/ml anti-A2B5 monoclonal antibody (1:200; R&D systems; 1:200) overnight at 4° C. Cells were then washed twice in PBS for 10 min and incubated with goat-anti-mouse Cy-3 second antibody (Jackson laboratories; 1:500) at room temperature for 20 min. The incidence of cells stained positive for A2B5 (an early marker of oligodendrocyte progenitors) was determined by using a fluorescent microscope equipped with Image ProPlus cell-counting program (Cybernetics).

Results:

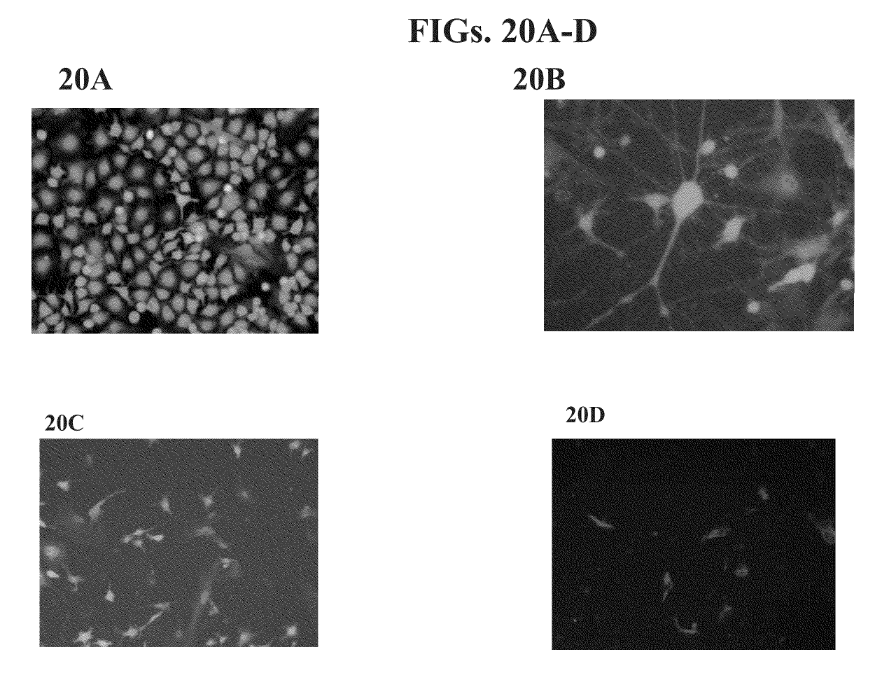

Neuronal cell morphology was observed in cells cultured with any one of the inducing substances alone (IL-1b, dbcAMP, retinoic acid, or NT-3) following 24 hr incubation. The most pronounced effect on cell-morphology was induced by dbcAMP and NT-3 (FIGS. 20A and 20B) as well as by IL-6 and thyroid factor 3 (data is not shown).

Cell survival was normal following 6 days incubation with any one of the substances alone (at either concentration) cell survival was normal. On the other hand, cell survival decreased when inducing substances were combined.

The incidence of cells stained positive for the oligodendrocyte progenitors marker A2B5 was 8% overall. The highest incidence of antibody-labeled A2B5 was found in the cells treated with NT-3 (FIGS. 20C-D).

Thus, BMSc can be induced to differentiate into precursors of oligodendrocytes (myelin producing cells), which may be utilized for treating multiple sclerosis.

### Example 13

### Cell Replacement in Amyotrophic Lateral Sclerosis (ALS)

Methods:

Animals:

TgN(SOD1-G93A)1Gur transgenic mice, expressing mutated human superoxide dismutase-1 gene (SOD1) (Gurney et al., 1994) were bred in CSJLF1. The transgenic mice were healthy until the age of 3 months then deteriorated with ALS and became completely paralyzed at the age of 4-5 month.

Transplantation:

Neuronal-differentiated male mouse BMSc were generated as described in Example 10 hereinabove. The cells were injected into the spinal cord (cisterna magna) of female mutant-SOD1 transgenic mice and of wild-type mice (10⁵ cells/injection; 5 animal replications per treatment group). Saline injections were used as control.

Motor Function Evaluation:

rotational behavior of treated and non-treated (saline only) mice was evaluated weekly by using a rotometer (San Diego Instrument Inc.).

Results:

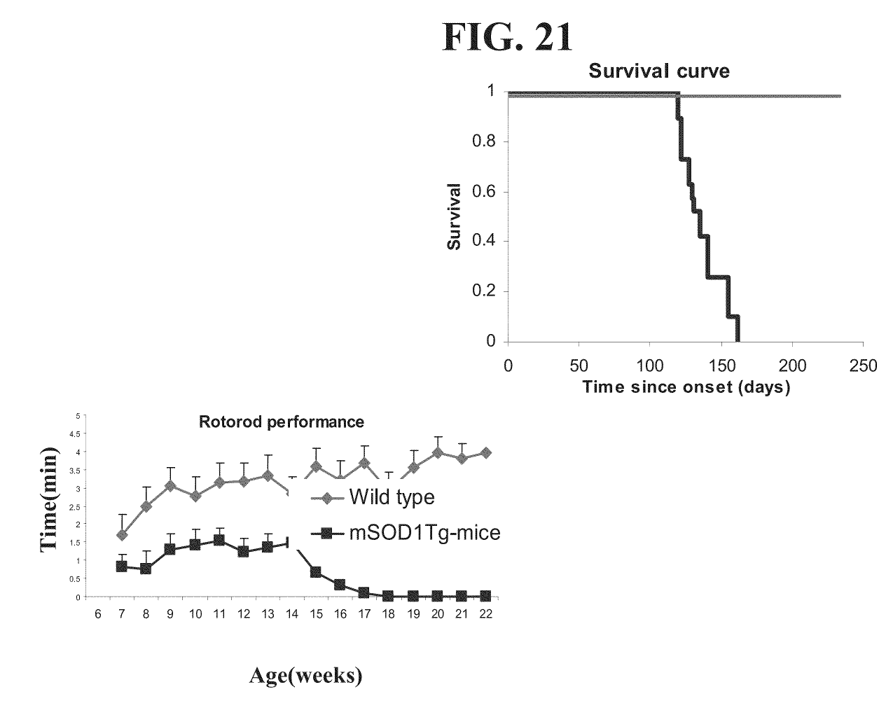

Mice expressing SOD1 suffered from amyotrophic lateral sclerosis (ALS) as indicated by a substantial reduction in rotational performance from week 7 onward, and a complete paralysis after 4-5 months (FIG. 21).

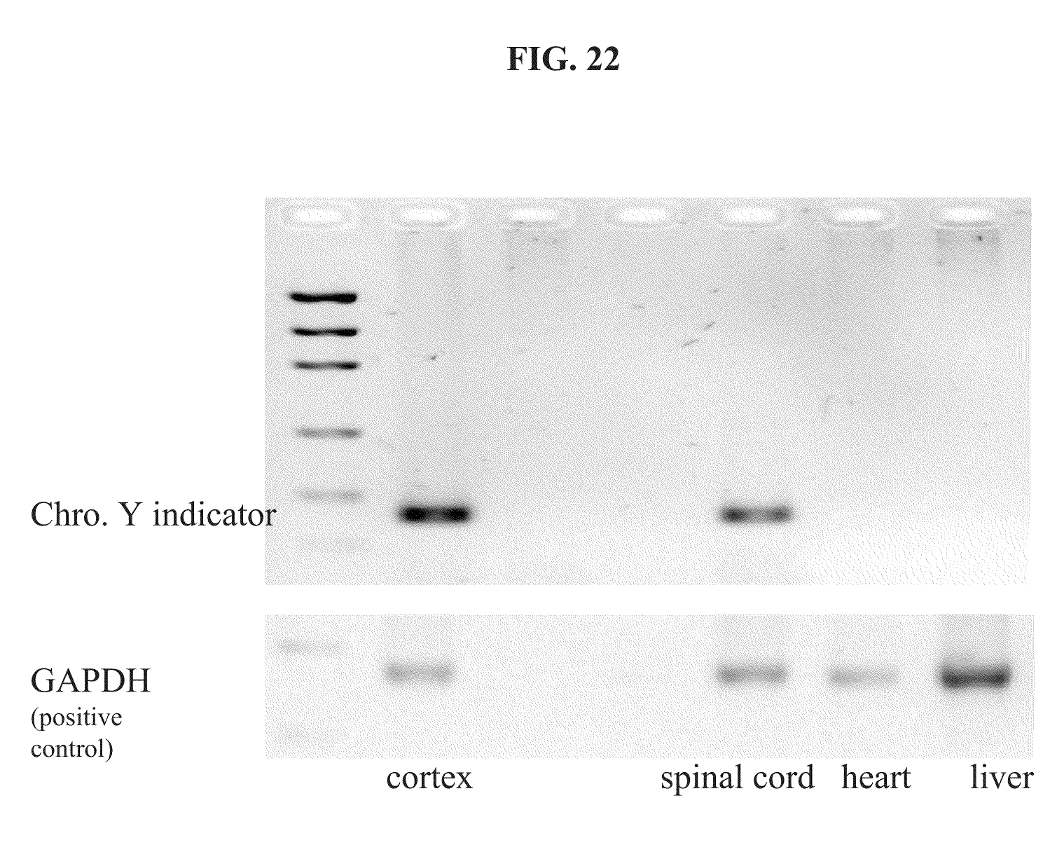

PCR analyses detected Y chromosome (indicative of male-derived transplanted cells) present the spinal cord of treated female mice. The Y chromosome was not detected in any other tissue of the treated female mice (FIG. 22).

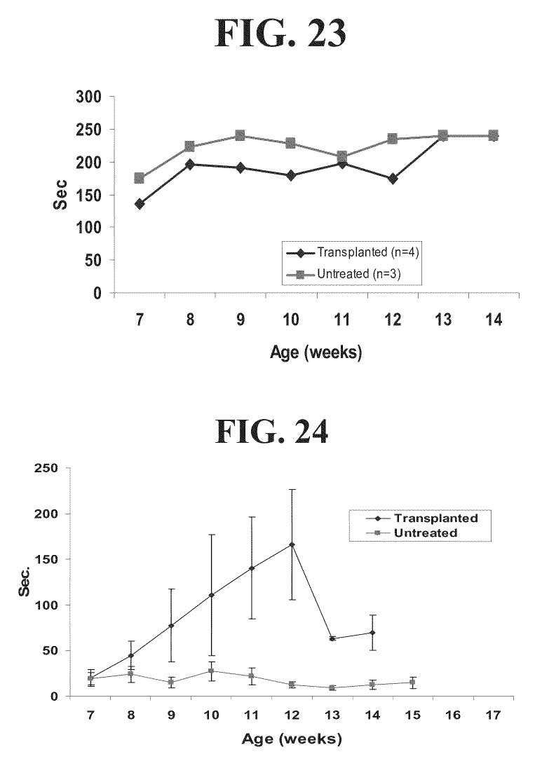

Rotational behavior of 7 week old treated wild-type was not significantly different from non-treated (saline only) wild type mice (FIG. 23). On the other hand, treated SOD1 mice exhibited substantial reduction in rotational behavior, indicating motor function improvement (FIG. 24).

### Example 14

### Construction of a Nurr1 Expression Vector

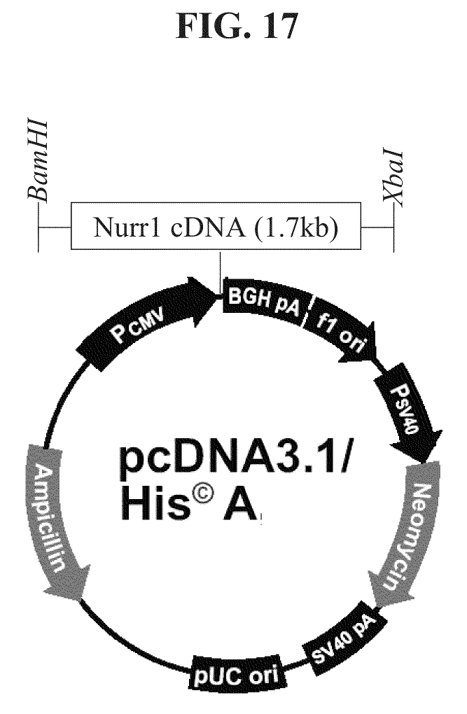

The nuclear receptor-related 1 (Nurr-1) is a transcription factor involved in differentiation of midbrain precursors into dopamine neurons A full-length human Nurr1 cDNA (GeneBank Accession No. NM_{—}173171) was amplified using primers 5′ BamHI and 3′ XbaI (primers set forth by SEQ ID NOs: 31-32) using high fidelity Taq polymerase (TaKaRa, Japan). The PCR condition of amplification were as follows: 10 cycles of 95° C., 1 min; 56° C., 1 min; 72° C., 1 min; 10 cycles of 95° C., 1 min; 55° C., 1 min; 72° C., 1 min; 10 cycles of 95° C., 1 min; 50° C., 1 min; 72° C., 1 min. The PCR products were digested with BamHI and XbaI restriction enzymes and the resulting fragments were inserted cloned using T4 DNA Ligase (New England BioLabs) into the expression vector pcDNA-3.1A (Invitrogene) as illustrated in FIG. 17.

Human bone marrow srtromal cells (hBMSc; 60-80% confluence) were transfected with pcDNANurr1 using FuGENE-6 transfection reagent according to the manufacturer's recommendations (Roche Applied Science). Stably transfected cells were isolated in a growth medium containing 500 μg/mL Neomycin (G418 Sulphate, Clontech, Palo Alto, Calif.). Total RNA was extracted from the isolated neomycin-resistant hBMSc as described by Chomczynski & Sacchi (1987) and the presence of Nurr1 transcripts was confirmed using the RT-PCR procedure as described in Example 3 hereinabove.

### Example 15

### Transforming hBMSc for Doxycyline-Regulated Expression of Tyrosine Hydroxylase

Inducible tyrosine hydroxylase (TH) expression can be effected by transforming hBMSc with a responsive and regulating vectors which can be constructed as follows:

Methods:

TH Responsive Vector:

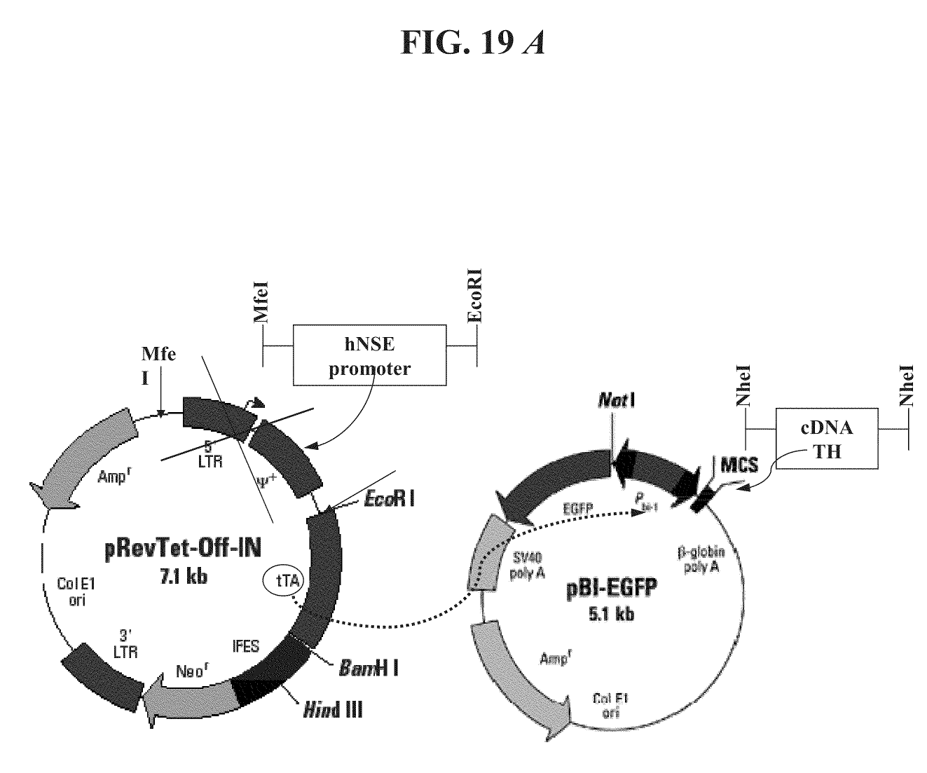

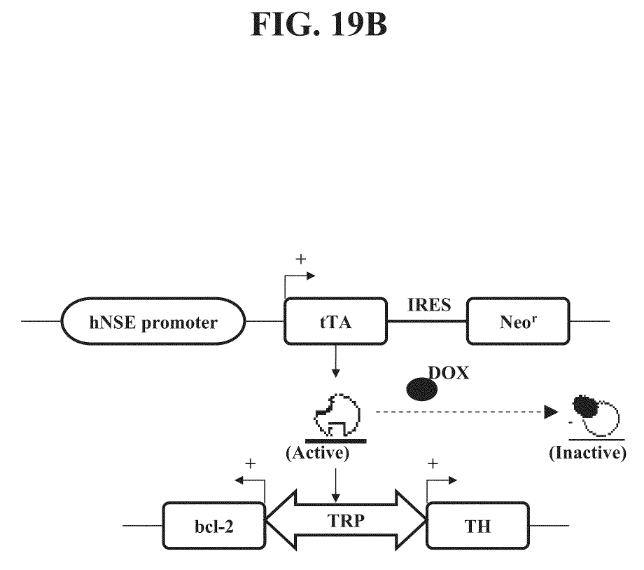

The 1.5 kb human tyrosine hydroxylase gene (TH, GenBank Accession No. NM_{—}000360 was isolated from human cDNA by PCR using high fidelity Taq polymerase (TaKaRa, Japan) the primers set forth by SEQ ID NOs: 39-40. The TH cDNA was inserted in pBI-EGFP (Clontech Tet-Off™ and Tet-On™Gene Expression Systems), as illustrated in FIG. 19A.

TH Regulating Vector:

The promoter of the 1.3 kb human NSE gene (HSENO2, GeneBank Accession No. X51956 was isolated from human cDNA by PCR using the primers of SEQ ID NOs: 37-38. The NSE-promoter cDNA was then inserted upstream of the transcriptional activator gene (tTA; Gossen, M. and Bujard, H. Proc. Natl. Acad. Sci. USA 89:5547-551, 1992) in pRevTet-Off-IN (Clontech), instead of the 5-LTR-Ψ⁺ as illustrated in FIG. 19A. The positive clones bearing the neo^{r} gene, were selected using the antibiotic neomycin.

hBMSc can be transformed with both response and regulator vectors (Tet-off/Tet-on system) by using any of the transformation methods described hereinabove. Once introduced into cells, the regulating vector which includes the internal ribosomal entry site (IRES) located between the tetracycline-controlled transactivator (tTA) and the gene encoding neomycin resistance (Ned), simultaneously expresses these two elements. The expressed tTA binds the tetracycline response element (TRE) located in the response vector, thereby activating transcription of TH. However, in the presence of doxycyline (a blood brain barrier traversing antibiotic) the binding of iTA to TRE is blocked thereby halting TH transcription. This drug-controlled expression of TH is schematically illustrated in FIG. 19B.

Hence, hBMSc can be genetically modified so as to express TH under the control of a negative regulator such as doxycyline which can be orally administered.

Since TH expression results in synthesis of dopamine, the genetically modified hBMSc can be used in cell replacement therapy to provide safe and effective treatment of neurodegenerative diseases such as Parkinson's disease.

It will be appreciated that positive regulation using an agent which induces TH expression can also be effected using for example, the Ecdysone-Inducible Mammalian Expression System (Invitrogen) utilizing the responsive vector pDHSP containing the TH gene and the regulator vector pVgRXR. In the presence of an inducer (e.g., ponasterone A or muristerone A) the functional ecdysone receptor binds upstream of the ecdysone responsive promoter and activates expression of TH.

### Example 16

### Intrastriatal Transplantation of Differentiated Mouse Bone Marrow-Derived Stem Cells Improves Motor Behavior in a Mouse Model of Parkinson's Disease

Differentiated mouse bone marrow-derived cells were transplanted into the striatum of 6-OHDA-lesioned mice; an animal model of PD. Survival and rotational behavior of the mice were analyzed.

Methods:

Isolation and Culture of mBMSc:

Mouse bone marrow stem cells (mBMSc) were obtained from the femur and tibia bone of Tg mice (B5/EGFP) bearing the enhanced green fluorescent protein (EGFP, Hadjantonakis, 1998) as described in Example 10 hereinabove. Cells were centrifuged and were plated in the proliferation medium (see in Example 1 hereinabove). Cells were incubated for 2 days and non-adherent cells were removed.

Differentiation:

As in table 1 of Example 2 (Levy et al., 2003).

6-Hydroxydopamine Lesion in Mice:

c57/bl male mice (˜30 gr) were anesthetized with chloral hydrate, 350 mg/kg intra-peritoneally (I.P.) and secured in a stereotaxic frame (Stoelting, USA). Mice were unilaterally injected with 6-OHDA hydrobromide (4 μg in 2 μl saline with 0.01% ascorbate, 1 μl/min). The coordinates of the striatum were: anterior 1.1 mm, lateral 2.3 mm, dorsa ventral 4.2 mm, with respect to bregma. 14 days following 6-OHDA injection, lesioned mice were tested for rotational behavior induced by an I.P. injection of amphetamine (10 mg/kg) for a period of 30 minutes.

Cell Transplantation:

Three weeks following the 6-OHDA lesion, 2×10⁵EGFP mouse bone marrow-derived stem cells differentiated for 48 hours were injected into the striatum of four lesioned mice that demonstrated amphetamine rotational behavior (>300 rotations/30 minutes).

Immunohistochemistry:

Immunohistochemistry was performed as described (see Example 5 of the Examples section). Sections were incubated with the primary antibody rat anti-TH (1:1000, v/v; Calbiochem) and the second antibody donkey anti rabbit AMCA/Cy3 (Jackson Lab.) as described in Tables 2-3 in Example 4 hereinabove. Transplanted cells were identified by immunostaining using goat anti-EGFP antibodies (1:200, v/v) followed by second antibody Donkey anti goat Cy2 as described in Example 11 hereinabove.

Rotational Analysis:

The rotational behavior 30 minutes following amphetamine challenge was measured, for a period of 30 minutes, every two weeks for three months. As a control, saline was injected into the striatum of four other lesioned mice.

Results

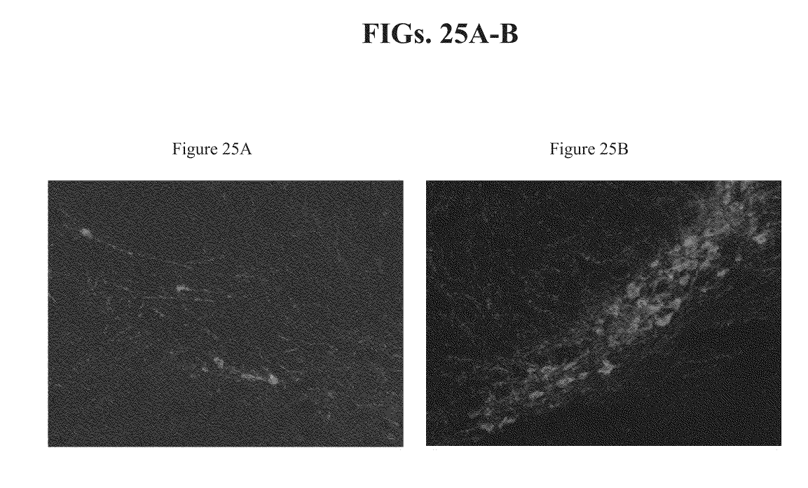

To examine the function of differentiated mouse bone marrow-derived stem cells in-vivo, the cells were transplanted intra-striatally in an animal model for Parkinson's, achieved by 6-OHDA injection into the striatum of C57/b mice. As seen in FIGS. 25A and 25B, this treatment markedly reduced the numbers of TH positive cells in the sustantia nigra, ipsilatteral to the lesion in the striatum.

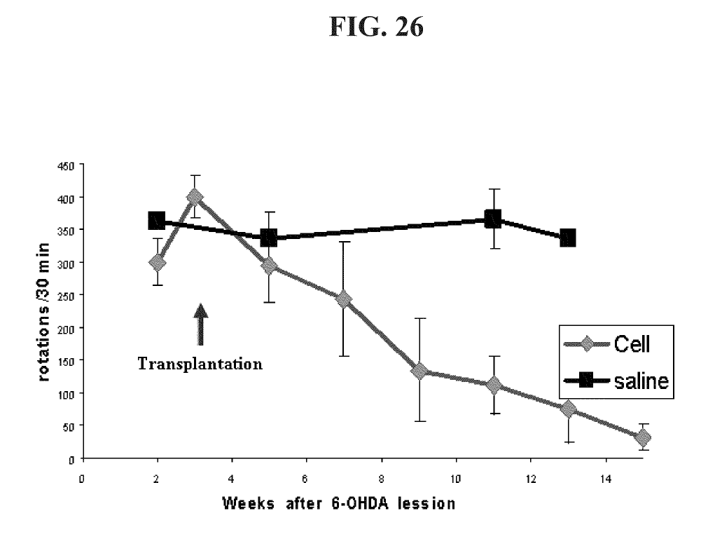

Measurements of the rotational behavior demonstrated a dramatic reduction in rotation number, forty five days following transplantation (FIG. 26).

The reduction in rotational behaviour was sustained and after twelve weeks, the cell-treated group reached the baseline level of rotations, prior to 6-OHDA lesion. In contrast, the rotations in the saline injected group remained unchanged.

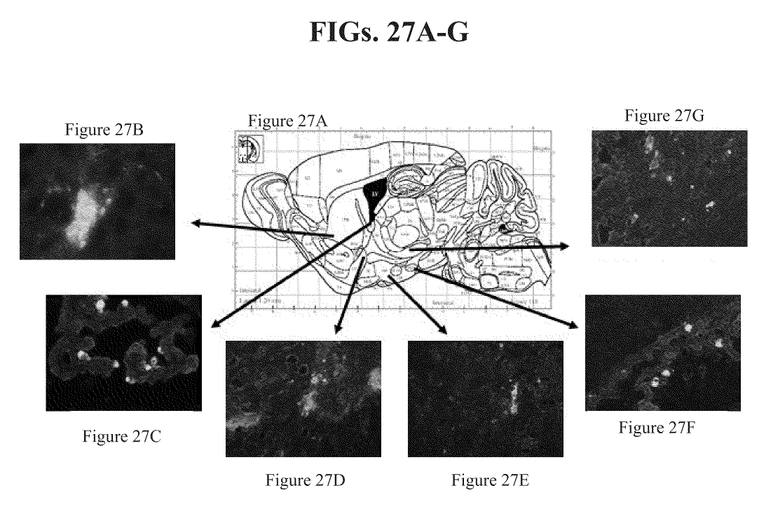

Histological analysis revealed that most of the EGFP-positive transplanted cells are located in the striatum, the injected area, while some of the cells migrated to the neighboring areas. The injected cells not only survived the transplantation, but migrated along the dopaminergic track, toward the subatantia nigra, and were observed in the striatum, nigrostriatal bundle ventral tegmental area (VTA) and the substantia nigra (FIGS. 27B-G). The cell number was counted in twelve slides and the average number is presented in Table 7 below. The estimated EGFP-positive cells in the various areas were calculated according to the volume of these regions, as estimated from the mouse brain atlas (Paxinos and Franklin, 2001).

TABLE 7
Analysis of cell staining with EGFP

| | Average | |
|---|---|---|
| | no of EGFP positive | Estimated total number of |
| Brain area | cells in 12 slides | EGFP positive cells |
| Striatum | 18 +/− 3 | 70,000 |
| Ventricle | 5.1 +/− 0.7 | 4750 |
| Thalamic nucleus | 5.6 +/− 1.6 | 14,500 |
| Substantia Nigra | 4.3 +/− 1.7 | 1,070 |

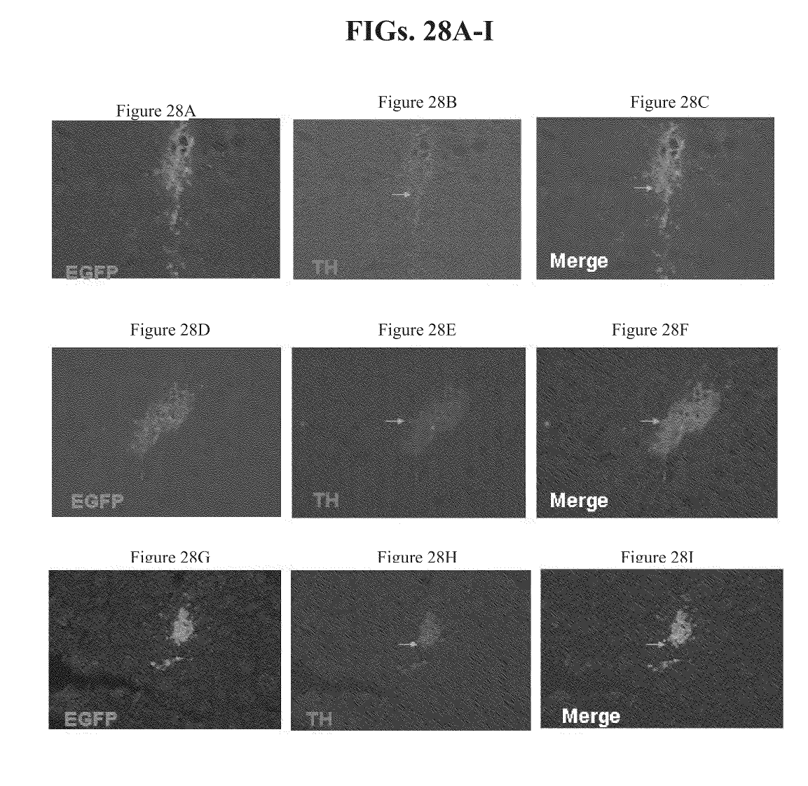

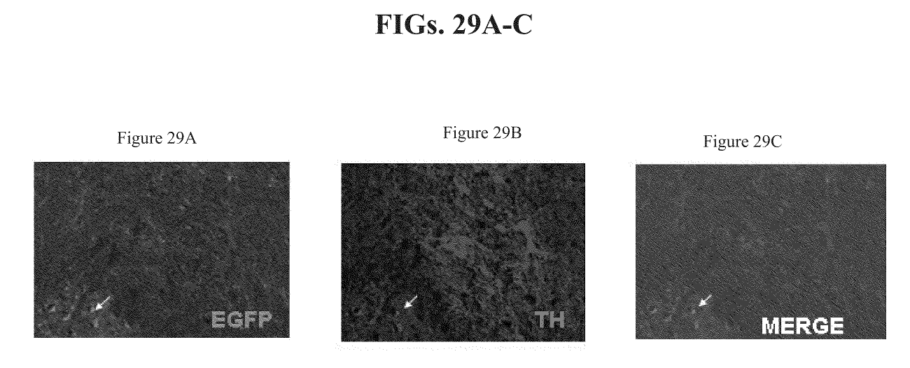

Double immuno-staining in the striatum revealed that some of the EGFP-positive transplanted cells, were also tyrosine hydroxylase positive, indicating the continuous expression of dopaminergic marker 12 weeks post-transplantation (FIGS. 28A-I). Immunostaining in the substantia nigra indicated the presence of TH positive bone marrow derived EGFP cells (FIGS. 29A-C).

Conclusion

Transplantation of the bone marrow-derived differentiated cells into the substantia nigra of 6-hydroxydopamine-lesioned mice was shown to reduce amphetamine-induced rotations. The rotational behavior completely ceased 45 days following transplantation. This beneficial effect was sustained for at least 4 months, when the animals were sacrificed. Immunostaining analysis of the transplanted mice' brains demonstrated that a subpopulation of the transplanted GFP-positive cells, express the dopaminergic marker, tyrosine hydroxylase. In conclusion, the above-described therapeutic modality support reduced PD symptoms in an animal model suggesting an accessible source of dopaminergic-like cells that may be used for treatment in Parkinson's disease.

### Example 17

### Survival of mBMSc Following Transplantation in the Right and Left Striata of 6-OHDA Unilateral Lesioned Mice

Methods:

6-Hydroxydopamine Lesion in Rats:

6-OHDA was injected into the right nigra of rats (Sprague-Dawley rats, 250 gr. n=6) using co-ordinates from the Stereotaxis Atlas: anterior 4.8 mm, lateral 1.8 mm, dorsoventral 8.1 mm, with respect to the bregma and the dura (Peng H, et al., 2004). Fourteen days following 6-OHDA injection, the lesioned rats were tested for rotational behavior induced by an intraperitoneal injection of amphetamine (10 mg/kg) for a period of one hour. Only rats with proven rotational behavior (>5 rpm) were selected for brain transplantation of mouse bone marrow cells.

Isolation and Culture of mBMSc:

Mouse bone marrow stem cells (mBMSc) were obtained from the femur and tibia bone of C57/Bl mice (B5/EGFP) as described in Example 10 hereinabove. Cells were centrifuged and were plated in the proliferation medium of Example 1 hereinabove but without the addition of non-essential amino acids and human epidermal growth factor (EGF). Cells were incubated for 2 days and non-adherent cells were removed.

Neural differentiation was then performed essentially incubating the cells for 24 hour in an additional differentiation medium followed by 48 hour incubation in differentiation medium as detailed below.

Additional differentiation medium: DMEM supplemented with 10% FCS, 10 ng/ml basic fibroblast growth factor, 10 ng/ml epidermal growth factor and 1% N2 solution.

Differentitation medium: DMEM supplemented with 200 μM butylated hydroxyanisole, 1 mM dibutyryl cyclic AMP, 0.5 mM isobutylmethlxanthine, 1% N2 solution, 10 μM retinoic acid and 100 μM ascorbic acid.

Following differentiation, approximately 10⁵ mouse bone marrow derived stem cells were injected into the right and left striata of 6-OHDA unilateral lesioned rats (n=4) using stereotactic frame (as described by Bjorklund et al., 2002).

Immunohistochemistry:

Immunohistochemistry of the rat tissue was performed 45 days following mouse bone marrow derived stem cell injection using rat anti-mouse antigen antibodies (M6, 1:200, v/v, Developmental Studies Hybridoma Bank, DSHB) followed by rabbit anti-rat HRP. Anti-tyrosine hydroxylase antibody was used as a neuronal specific antibody to detect the presence of neurons.

Results

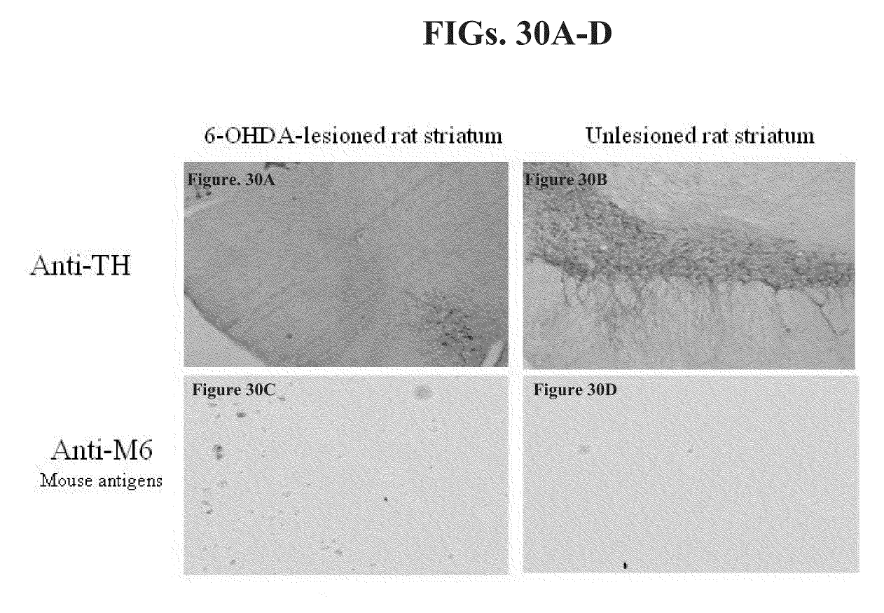

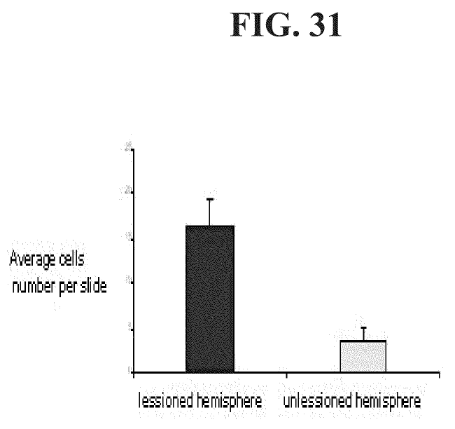

The 6-OHDA unlesioned hemisphere contained intact TH positive neurons (as seen in FIG. 30B) but showed only weak staining using M6 antibody (FIG. 30A). In contrast, there was an absence of TH positive neurons in the lesioned hemisphere (FIG. 30D), but strong staining was detected with the M6 antibody (FIG. 30C). Counting of the M6-immunopositive cells demonstrated significantly higher survival of cells in the right hemisphere, the 6-OHDA injected side, as compared to the left, unlesioned hemisphere (FIG. 31). Thus, the unilateral 6-OHDA lesion in the nigra followed by distraction of the dopaminergic terminals in the striatum increased the survival of the engrafted cells.

### Example 18

### The Role of Damaged Striata in Attracting Transplanted Cells to the Lesion

To address the question whether the damaged striata might release factors that attract the transplanted cells to the lesion, 6-OHDA was injected into the right striatum of the mouse brain while the cells were transplanted into the left striatum.

Methods:

6-Hydroxydopamine Lesion in Mice:

c57/bl male mice (˜30 gr) were anesthetized with chloral hydrate, 350 mg/kg intra-peritoneally (I.P.) and secured in a stereotaxic frame (Stoelting, USA). Mice were unilaterally injected with 6-OHDA hydrobromide (4 μg in 2 μl saline with 0.01% ascorbate, 1 μl/min). The coordinates of the striatum were: anterior 1.1 mm, lateral 2.3 mm, dorsa ventral 4.2 mm, with respect to bregma. 14 days following 6-OHDA injection, lesioned mice were tested for rotational behavior as described in Example 10. Only mice with proven rotational behavior (>5 rpm) were selected for brain transplantation. As controls, naive mice were also transplanted with BMSc.

Isolation and Culture of mBMSc:

Mouse bone marrow stem cells (mBMSc) were obtained from the femur and tibia bone of Tg mice (B5/EGFP) bearing the enhanced green fluorescent protein (EGFP, Hadjantonakis, 1998) as described in Example 10 hereinabove and approximately 0.2×10⁶ cells were transplanted twenty days following the lesion. Both differentiated and non-differentiated mBMSc were transplanted (6 mice per group). The mBMSc differentiation procedure was performed as described in Example 17, apart from the fact that twenty four hours prior to the transplantation, cells were transfected with (5 mg/ml) iron using Fugene reagent (1 ml/ml) in DMEM medium.

Fluorescent Microscopy:

45 days following mBMS cell injection, tissue sampled from lesioned and non-lesioned mouse hemispheres were sectioned and observed under a fluorescent microscope for the presence of green fluorescent protein (GFP) marking the transplanted mBMSc.

Detection of Iron-Transfected Cells:

To ascertain the presence of the transplanted mBMSc by detecting the iron-transfected cells in-situ, slides were placed in 4% potassium ferrocanide with an equal volume of 1.2 mmol/L hydrochloride acid solution (Sigma MA-HT20) for 10 minutes, rinsed in deionized water and then stained with pararosaniline solution for 3-5 minutes.

Results

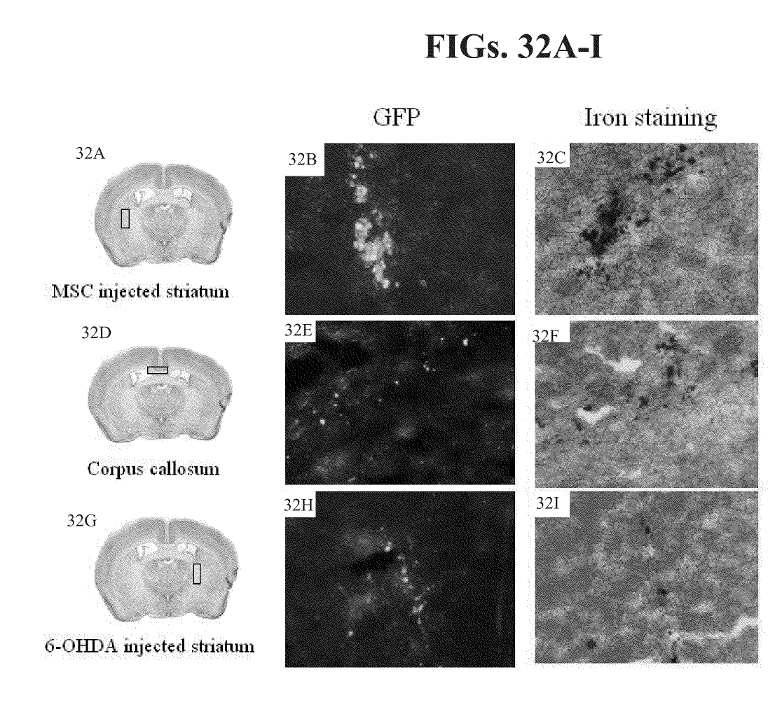

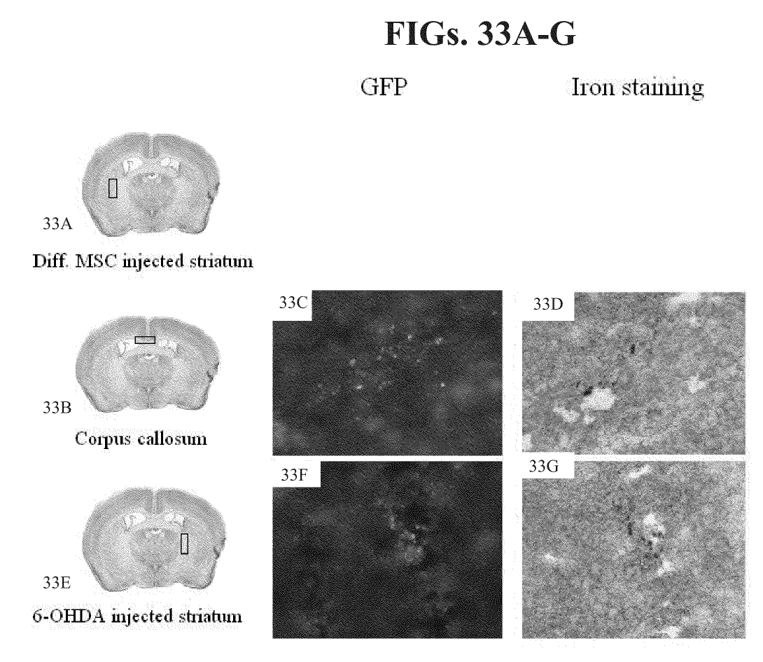

Histological studies demonstrated that most of the GFP-positive cells were located in the injected area in the striatum (FIG. 32B). However, transplanted cells were clearly seen in the collateral striatum around the 6-OHDA lesion (FIGS. 32H and 33F). Moreover, GFP cells were found along the path of the migration from the left striatum, through the corpus callosum, ending in the right striatum, thalamic nuclei and substantia nigra (FIGS. 32E and 33C).

The iron staining also demonstrated the accumulation of iron-positive cells in the injected striatum (FIG. 32C), but a significant amount of cells were detected in the contralateral striatum around the 6-OHDA lesion (FIGS. 32I and 33G) and the path of the migration (FIGS. 32F and 33D), similar to the GFP labeling. Both MSC and neuronal-differentiated MSC were seen to migrate similarly and populate the 6-OHDA lesioned hemisphere.

### Example 19

### Bone Marrow Derived Cells Exhibit Mesenchymal Characteristics Following Incubation in Proliferation Medium

Bone marrow derived mesenchymal stem cells can be distinguished from hematopoietic stem cells by their plastic-adherence quality. In an effort to further characterize the cells, the expression of a wide selection of hematopoietic and mesenchymal markers was examined by FACS analysis.

Methods:

Isolation and Culture of Human BMSc:

Human bone marrow mesenchymal cells (hMSCs) were collected from iliac crest of healthy human donors ranging in age from 19 to 76 years. Primary cultures of hBMSc were obtained as described by Schwarz et al., 1999. Bone marrow aspirates (10 ml) were obtained from each donor, and diluted with 10 ml of Hank's balanced salt solution (HBSS; Biological Industries, Bet-Haemek, Israel). Mononuclear cells were isolated by centrifugation at 2500 g for 30 minutes at room temperature through a Ficoll density gradient (Histopaque®1077; Sigma) or in UNISEP-MAXI tubes (Novamed, Jerusalem, Israel) on the basis of density gradient. The mononuclear cell layer was recovered from the gradient interface, washed with HBSS and centrifuged at 2000 g for 20 min at room temperature. A portion of these cells were taken for FACS analysis, while the remaining cells were plated in 75-cm² polystyrene plastic tissue-culture flasks (Corning, Corning, N.Y.) in the proliferation medium described in Example 1. Non-adherent cells were removed following 48 hours, and the medium was replaced every 3-4 days.

Flow Cytometry:

MSCs were harvested from the tissue culture flasks on days 14, 23, 31 or 33 by incubation in trypsin-EDTA (Beit-Haemek) in 37° C. followed by neutralization by the addition of 10 ml of fresh medium. Cells were centrifuged at 1000 g for 10 minutes, room temperature. The pellet was resuspended in PBS and divided into duplicate samples in 50 ul PBS. An isotype control was included in each experiment to identify background fluorescence. The cells were incubated with the appropriate antibodies for 45 minutes on ice, washed twice in flow-buffer (5% FCS, 0.1% sodium-azide in PBS), and centrifuged at 1000 g for 10 minutes. The cells were resuspended in 0.5 ml PBS and analyzed by FACS-Calibur™ flow cytometer using the Cellquest software program 3.0 (Becton Dickinson). A minimum of 10,000 events were examined per sample.

The Following Antibodies were Used for FACS Analysis:

Phycoerythrin (PE) conjugated Mouse IgG1 anti-human CD29 (Integrin β₁) (1:25; eBioscience), PE conjugated Mouse IgG1 anti-human CD19 (1:10; eBioscience), PE conjugated Mouse IgG1 anti-human CD44 (1:10; Cymbus Biotec), PE conjugated Mouse IgG1 anti-human CD56 (1:20; BD Biosciences) and PE conjugated Mouse IgG1 Isotype (1:10; eBioscience) was used as control. Fluorescein isothiocyanate (FITC) conjugated Mouse IgG2a anti-human CD45 (Leukocyte common antigen Ly-5; 1:10; Miltenyi Biotec), FITC conjugated Mouse IgG2a anti-human CD34 (1:10; Miltenyi Biotec), FITC conjugated Mouse IgG2k monoclonal anti-human CD105 (Endoglin) (1:100; Ancell.Co) and FITC conjugated Mouse IgG1 Isotype (1:10; eBioscience) was used as control.

Differentiation to Adipocyte and Osteoblasts:

Adipogenic differentiation was achieved by culturing hMSCs in DMEM supplemented with 10% FBS, 100 units/ml penicillin, 100 μg/ml streptomycin, 1 mM L-glutamine, isobutyl methylxanthine (IBMX; Sigma), and 60 μM indomethacin (Sigma) with medium replacement every 4-5 days for 12-21 days. Detection of adipocytes was achieved by staining for 30 minutes with Oil Red O (prepared by adding 3 parts of 0.5% Oil Red O in isopropanol to 2 parts of ddH₂O and filtering the solution through a 0.2-μm filter; Sigma). The cells were then washed twice with PBS, and the lipid vacuoles were identified as bright red inclusions within the cells.

For osteogenic differentiation, hMSCs were cultured in medium containing DMEM supplemented with 10% FBS, 100 units/ml penicillin, 100 μg/ml streptomycin, 1 mM L-glutamine, 1×10⁻⁸ M dexamethasone (Sigma) and 50 μM ascorbic acid (Sigma), with medium replacement every 4-5 days for 12-21 days. The cells were then stained with 0.5% alizarin red S for 5-10 minutes (adjusted to pH 4.1 by 0.5% KOH solution; Sigma). The plates were washed at least three times with water, and the mineral deposits were seen as dark red stains within the cells.

Results

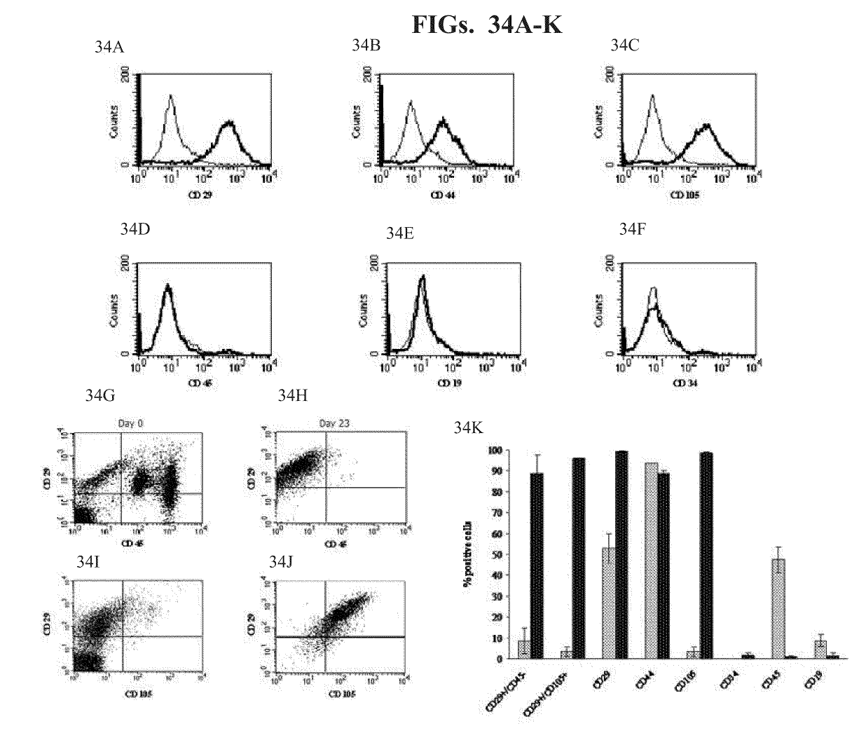

The levels of mesenchymal marker CD29 increased from 52.81±12.07 percent on day 0 to 99.20±0.74 percent from day 14 onward (FIGS. 34A and 34K). Moreover, levels of mesenchymal marker CD105, increased from 3.51±3.24 percent on day 0 to 98.69±0.77 percent from day 14 onward (FIGS. 34C and 34K). CD44, another mesenchymal antigen, was highly expressed on day 0 (93.75 percent) and remained high on day 14 and onward 88.63±3.31 (FIGS. 34B and 34K). In addition, approximately 77 percent of the cells expressed CD90 following day 1.

The levels of hematopoietic marker CD45 decreased from 47.53±10.70 percent on day 0 to 1.01±0.94 percent from day 14 onward (FIGS. 34D and 34K), as did levels of CD19, which decreased from 8.84±3.85 percent on day 0 to 1.41±2.83 percent from day 14 onward (FIGS. 34E and 34K). A very low level of CD34 was expressed (1.88±1.68 percent) from day 14 on (FIGS. 34F and 34K). No significant expression of CD20, CD5, CD11B or FMC7 was found.

In addition, double staining of the cells showed that on day 0 only 8.66±0.56 percent of the cells were CD29+/CD45− (FIG. 34G), and a mere 3.51±3.24 percent were CD29+/CD105+ (FIG. 34I). However, following 14 days in vitro, 88.84±12.01 percent of the cells were positive for mesenchymal marker CD29, and negative for hematopoietic marker CD45 (FIG. 34H), and 96.15±1.48 percent of the population was positive for both mesenchymal markers CD29 and CD105 (FIG. 34J), confirming that the majority of the cells express more than one mesenchymal marker, and do not express hematopoietic markers.

Conclusions

FACS analysis of the cells showed a significant increase in expression of mesenchymal markers (CD44, CD29, CD105 and CD90), while a reduction in the expression of hematopoietic markers (CD45, CD34, CD19, CD20, CD5, CD11B, FMC7) to negligible levels was observed by day 14 in vitro. The levels of the examined markers remained stable at all times measured (days 14-36 in vitro).

Thus, it can be concluded that over time in culture, the mesenchymal subpopulation of the bone marrow was selected for, and enriched by conditions in vitro. After 14 days in vitro, the majority of the plastic-adherent population consists of cells presenting a stable antigenic profile of CD105⁺/CD29⁺/CD44⁺/CD90⁺/CD34⁻/CD45⁻/CD19⁻/CD5⁻/CD20⁻/CD11B⁻/FMC7⁻ which is a distinct phenotype of MSCs.

Further traits of the cells, typical for mesenchymal stem cells, include cell morphology which remained spindle-like throughout the time in vitro. Moreover, the cells formed single-cell derived colonies and readily differentiated into adipocytes and osteoblasts when exposed to appropriate differentiation conditions. Thus, it can be concluded that the morphology, clonality, differentiation potential and membrane markers indicate a mesenchymal stem cell identity of the cell population.

### Example 20

### Naïve Undifferentiated Human Mesenchymal Stem Cells Express Neural Genes and Transcription Factors

Mesenchymal stem cells may potentially differentiate into neurons in vivo and in vitro. In an effort to gain a better understanding of mesenchymal stem cell plasticity, and, more specifically, of the neural potential of these cells, naive undifferentiated hMSCs were examined for the expression of those neural-specific genes and those genes of the dopaminergic system which were shown to be up-regulated following the induction of neuronal differentiation.

Methods:

Isolation and Incubation of Human BMSCs:

Human BMSCs were isolated as described in Example 1 and incubated in the proliferation medium as described in Example 1.

Isolation and Incubation of Mouse BMSCs:

C3H.sw and C57bl/J6 mice were obtained from Harlan laboratories Ltd. Mouse BMSCs were isolated as described in Example 17 and incubated in the proliferation medium as described in Example 1.

Neuronal Differentiation of Mouse and Human BMSCs:

Human BMSCs were differentiated as described in table 1 of Example for up to 96 hours. The cells exhibited morphological features typical of neurons such as refractile cell bodies and long branching processes with growth cone-like terminal structures. The acquired neuronal phenotype was analyzed by detection of neuronal mRNA and proteins, as described below.

Protein/DNA Array Analysis:

Nuclear proteins were isolated from hBMSC 24 and 48 hours following incubation in the neuronal differentiation medium described above. The nuclear proteins were extracted using nuclear extraction kit (Panomics Inc.) following the manufacturer's instructions.

The nuclear proteins extracted from each type of cells were incubated with the TranSignal probe mix (a set of 96 biotin-labeled DNA binding oligonucleotides corresponding to the consensus sequences of 96 transcription factors as detailed in Table 8 below, respectively; Panomics Inc.) to allow the formation of DNA/Protein complexes. The complexes were then separated from the free probes by agarose gel electrophoresis. The probes in the complexes were extracted from the gel, dissociated from the DNA/Protein complexes, and used to hybridize the TranSignal Array membrane spotted with the same consensus-binding sequences of the 96 transcription factors (Panomics Inc.). Hybridization signals were based on HRP chemiluminescence and exposed to X-ray film. Densitometry was measured by the Versa Doc imaging system (BIO RAD). The intensity of the signal is an average of duplicate spots on the array membrane. All transcription factors that exhibited intensities of less than 0.5 ODu*mm² in both type of cells were eliminated. For each remaining transcription factor, the ratio of undifferentiated/neuronally-differentiated level was calculated. In accordance with Panomics instructions, a ratio between 0 and 0.5 or 2.0 and above represented a significant difference in transcription factor levels.

Isolation and Preparation of RNA:

Total RNA was extracted from untreated hMSCc, neuronally differentiated hMSCs and human lymphocytes (as a negative control) by using the guanidine isothiocyanate method (Chomczynski & Sacchi 1987). RNA was quantified by spectrophotometer (Uvikon 860; Tegimenta AG Instruments, Switzerland), and separated by 1% agarose formaldehyde-denaturing gel electrophoreses (Sigma) to verify its integrity. Reverse transcription (RT) was carried out on 0.05 μg/μl mRNA samples using the 5 units/μl enzyme SuperScript™ II RNase H⁻ Reverse Transcriptase in a mixture containing 2 μM random primers (mostly hexamers), 10 mM dithiotheitol (DTT), 1× buffer supplied by the manufacturer (Invitrogen™ Life Technologies, Carlsbad, Calif.), 20 μM dNTPs (TaKaRa Biotechnology, Gennevilliers, France), and 1 unit/μl RNase inhibitor (RNAguard, Amersham Biosciences, Buckinghamshire, England). The RT was performed at 25° C. for 10 min, 42° C. for 120 min, 70° C. for 15 min, and 95° C. for 5 min.

Polymerase Chain Reaction (PCR):

PCR amplifications were performed in a 20 μl final volume containing 2 μl of reverse-transcribed RNA (cDNA), 0.5 μM of sense and anti-sense primers, 1× buffer supplied by the manufacturer, 225 μM dNTPs, Taq DNA polymerase 1 unit (TaKaRa). Primers were chosen from different exons to ensure that the PCR products represent the specific mRNA species and not genomic DNA. PCR conditions were optimized by varying the cycle numbers to determine a linear amplification range. cDNA underwent up to 50 cycles of amplification (1 min at 94° C., 1 min at 54-65° C. and 1 min at 72° C.) in PCR set PTC-100™ Research, Waltham and Watertown, Mass.). The PCR reaction was resolved on a 1% agarose gel. The bands were observed under UV light and photographed (VersaDoc™ model 1000 Imaging System, Bio-Rad Laboratories, Hercules, Calif.).

Primer sequences are detailed in Table 2 as set forth by SEQ ID NOs: 3-8, 11,12,15,16,19-22 and 39-66.

Northern Blot Analysis:

All reagents/materials were obtained from Sigma. 10 μg of total RNA from untreated and neuronally differentiated hBMSc were fractionated on 1% agarose containing 3% formaldehyde and 1×MOPS buffer [20 mM 3-(N-morpholino) propanesulfonic acid (pH 7.0), 5 mM sodium acetate, and 1 mM EDTA] by denaturing gel electrophoresis. RNA was then transferred to Duralon-UV™ membranes (Stratagene, Cedar Creek, Tex.) by upward capillary transfer, which cross-linked by 1200 mJ per cm² UV radiation (Hoefer Scientific Instruments, San Francisco, Calif.). Positions of 28S and 18S ribosomal RNA were marked after transfer. Membranes were prehybridized in a mixture of 50% formamide, 5×Denhardt's solution, 200 μg/ml salmon sperm DNA (SSDNA), 1×SSPE (1 SSPE=0.15M NaCl, 10 mM NaH₂PO₄, 1 mM EDTA, pH 7.4) and 0.1% sodium dodecyl sulfate (SDS) at 42° C. for 1-2 hr. cDNA was generated by RT-PCR reaction and probes for human neurite outgrowth-promoting protein (NEGF) neurofilament-200 (NEF-H), neuron specific enolase (NSE), and glyceraldehydes 3-phosphate dehydrogenase (GAPDH) were prepared by the specific primers as detailed in Table 2 in PCR apparatus. Probes were labeled with ³²P-dCTP (NEN Life Science Products, Boston, Mass.) and Klenow enzyme (New England BioLabs, Beverly, Mass.). The labeled probes were heat-denatured (90° C. for 5 min, and ice for 5 min), and was added to the prehybridization solution and hybridized at 42° C. for 18 hr with the membrane. After hybridization, the membranes were washed twice with 0.1×SSC (1×SSC=0.15M NaCl, 15 mM sodium citrate, pH=7) and 0.1% SDS at room temperature for 20 min, and twice with the washed solution at 65° C. for 30 min, and exposed to storage phosphor screen for various intervals. The hybridization signals were measured with a phosphor-imager (Cyclone, Packard, UK) and analyzed with OptiQuant™ software. After autoradiography, membranes were stripped and re-hybridized with a ³²P-dCTP-labeled probe for GAPD, a housekeeping gene, to normalize total RNA levels and verify equal loading and transfer of RNA.

Western Blot Analysis:

Protein extracts from hBMSc and differentiated hBMSc were prepared in 50 μl of cold buffer containing 105 mM Tris (Sigma), 5 mM EDTA (BDH Laboratory Supplies, Poole, England), 140 mM NaCl (BioLab, Jerusalem, Israel), 10 mM sodium fluoride (Sigma), 0.5% NP-40 (United States Biochemical Corporation, Cleveland, Ohio), 1 μM PMSF (Sigma). Homogenates were centrifuged at 13000 g for 20 min at 4° C., and supernatants were collected. Protein concentration was determined and 80 μg samples diluted 1:5 with sample buffer (62.5 mM Tris-HCl, pH 6.8, 10% Glycerol, 2% sodium dodecyl sulfate, 5% 2-β-mercaptoethanol, 0.0025% bromophenol blue, Sigma) and boiled for 5 minutes heated prior to loading. Proteins were size fractionated on 12.5% SDS-polyacrylamide gels (Bio-Rad Laboratories, Hercules, Calif.) and electroblots were transferred to polyvinylidene difluride membranes (Bio-Rad Laboratories). The membranes were probed with primary antibodies rabbit anti human:Nestin (1:2500, kindly provided by C. A. Messam, National Institute of Neurological Disorders and Stroke, Bethesda, Md.); TH (1:6000, Chemicon, Temecula, Calif.). Primary antibodies mouse anti human: Neuronal nuclei (NeuN, 1:1000), NSE (1:750), and actin (1:1250, Chemicon) was used to evaluate and quantify the changes during the induction of neural differentiation. Membranes were then exposed to horseradish-peroxidase conjugated goat anti-rabbit IgG diluted at 1:25000, or anti-mouse IgG diluted at 1:20000 (Jackson ImmunoResearch Laboratories, West Grove, Pa.), for 30 min at room temperature. The membranes were then stained using the enhanced SuperSignal® chemiluminescent detection kit (Pierce) and exposed to medical X-ray film (Fuji Photo Film, Tokyo, Japan). Densitometry of the specific proteins bands was preformed by VersaDoc® imaging system (Bio-Rad Laboratories) and Quantity One® software (Bio-Rad).

Immunocytochemistry:

hBMSc were plated and treated in slides chamber (Nalge Nunc International, Naperville, Ill.) previously treated with 10 μg/ml human fibronectin (Chemicon). Cells were fixed with 4% paraformaldehyde and permeabilized with PBS containing 0.1% Triton X-100 (Sigama) and 10% goat serum (to block non-specific binding sites, Biological Industries). The differentiated hBMSc were stained with the following antibodies. Rabbit antibodies against human:nestin (1:200). Mouse antibodies against human:NeuN (1:50), NEF-H (1:200, Sigma), NSE (1:100). Appropriate cyanin-2 (Cy2) and Cy3-labeled secondary antibodies (Jackson ImmunoResearch) and DNA-specific fluorescent dye 4,6-diamidino-2-phenylindole (DAPI; Sigma) counterstains were used for visualization. Cells were photographed on a fluorescence Olympus IX70-S8F2 microscope with fluorescent light source and a U-MNU filter cube (Olympus). The staining cells were counted (5 random frames per well) using the Image ProPlus (Media Cybernetics, Silver Spring, Md.) cell-counting program.

Flow Cytometry:

Flow cytometry was performed as described in Example 19 of the Examples section herein above. For labeling of Nestin in hMSCs Mouse IgG anti Nestin (1:20; R&D systems) was used as the first antibody and anti mouse Alexa488 (1:500; Molecular Probes) as the secondary antibody.

Statistical Analysis:

Each value is the mean±S.E.M. of more than two independent experiments. Statistical significance for comparisons among groups was determined by using two-tail unpaired Student T-test. In all tests, significance was assigned when p<0.05.

Results

From the RT-PCR analysis, Western blot analyses, Northern Blot analyses and immunohistochemistry analyses, it was found that most of the examined genes were expressed in the naive mesenchymal stem cells (11/15 neural genes, 12/12 neural transcription factors, 4/4 dopaminergic transcription factors and 4/8 dopaminergic genes as detailed in Table 8 herein below).

TABLE 8
Analysis of neural specific and dopaminergic genes in undifferentiated hBMSc

| | | | |
|---|---|---|---|
| | | Cell | |
| Gene name | Expression | type | Methods |

NEURAL GENES

| | | | |
|---|---|---|---|
| 2′,3′-Cyclic nucleotide 3′-phosphodiesterase | + | Human, | Western blot |
| (CNPase) | | Mouse | Immunochemistry |
| Glypican-4 (GPC4) | + | Human | RT-PCR |
| Necdin | − | Human | RT-PCR |
| Nestin | + | Human | RT-PCR |
| | | | Western blot |
| Neurite growth-promoting factor 2 (NEGF-2) | + | Human | Northern blot |
| Neurofilament-Heavy (NF-H 200 kDa) | + | Human | Northern blot |
| Neurofilament-Light (NF-L 70 kDa) | − | Human | RT-PCR |
| Neurofilament-Medium (NF-M 160 kDa) | + | Human | RT-PCR |
| Neuron specific enolase (NSE) | + | Human | RT-PCR |
| | | | Western blot |
| | | | Northern blot |
| Neuronal Nuclei (NeuN) | + | Human | RT-PCR |
| | | Mouse | Western blot |
| Neurotrophic tyrosine kinase receptor type 2 | + | Human | RT-PCR |
| (TRK-2) | | | |
| RET tyrosine kinase | − | Human | RT-PCR |
| Retinoic acid receptor type a (RARA) | + | Human | RT-PCR |
| Tryptophan hydroxilase (TPH) | − | Human | Western blot |

NEURALLY ACTIVE TRANSCRIPTION FACTORS:

| | | | |
|---|---|---|---|
| Aryl hydrocarbon receptor/Aryl hydrocarbon | + | Human | Protein/DNA array |
| receptor nuclear translocator binding element | | | |
| (AhR/Arnt) | | | |
| Ecotropic viral integration site 1 (EVI-1) | + | Human | Protein/DNA array |
| Forkhead box O1A human (FKHRhu) | + | Human | Protein/DNA array |
| Glycosaminoglycan (GAG) | + | Human | Protein/DNA array |
| Hepatocyte nuclear factor 3β (HNF-3β) | + | Human | Protein/DNA array |
| Myelin gene expression factor 2 MEF2(2) | + | Human | Protein/DNA array |
| Nuclear Y box factor (NF-Y) | + | Human | Protein/DNA array |
| Neural zinc fingure 3 (NZF-3) | + | Human | Protein/DNA array |
| Paired box gene 3 (Pax-3) | + | Human | Protein/DNA array |
| Paired box gene 6 (Pax-6) | + | Human | Protein/DNA array |
| Xenobiotic response element (XRE) | + | Human | Protein/DNA array |

DOPAMINERGIC TRANSCRIPTION FACTORS:

| | | | |
|---|---|---|---|
| Engrailed 1(En-1) | + | Human | RT-PCR |
| Nurr-1 | + | Human | RT-PCR |
| Paired-like homeodomain transcription factor 3 | + | Human | RT-PCR |
| (PITX-3) | | | |

DOPAMINERGIC GENES:

| | | | |
|---|---|---|---|
| Aldehyde dehydrogenase 1 (Aldh1) | + | Human | RT-PCR |
| Aromatic L-amino acid decarboxylase (AADC) | + | Human | RT-PCR |
| Catechol-o-methyltransferase (COMT) | + | Human | RT-PCR |
| Dopamine transporter (DAT) | − | Human | RT-PCR |
| Dopamine receptor D2 (DRD2) | − | Human | RT-PCR |
| GTP cyclohydrolase-1 (GCH) | + | Human | RT-PCR |
| Monoamine oxidase B (MAO-B) | − | Human | RT-PCR |
| Patched homolog(PTCH) | + | Human | RT-PCR |
| Smoothened (SMO) | + | Human | RT-PCR |
| Tyrosine hydroxilase (TH) | + | Human | Western blot |
| Vesicular monoamine transporter 2 (VMAT 2) | − | Human | RT-PCR |

RT-PCR:

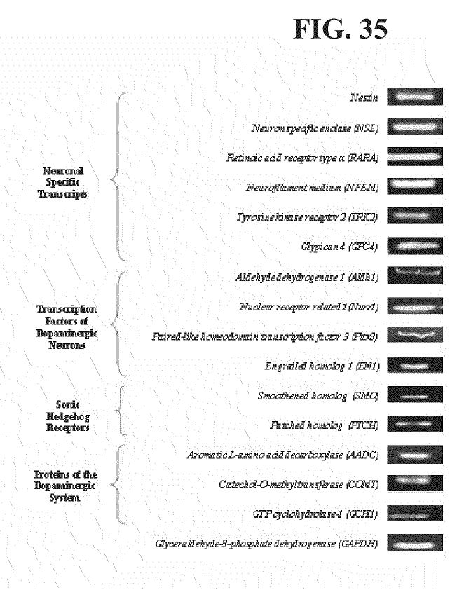

Results from the RT-PCR analysis are illustrated in FIG. 35. Of note, all of the midbrain dopaminergic transcription factors (Nurr1, Pitx3 and En-1) were found to be significantly expressed by non-differentiated hMSCs. The expression of Nurr1 remained high throughout the neuronal differentiation process. Transcripts of nestin, neuron specific enolase (NSE), neurofilament-medium (NF-M), Retinoic acid receptor-type α (RARA) and GPC4 were detected (FIG. 35). Transcripts of the following neural genes were not detected: Necdin, neurofilament-light (NF-L), RET tyrosine kinase and vesicular monoamine transporter 2 (VMAT 2). PTCH and SMO, (two genes associated with the action of an important modulator of the CNS formation) were both found to be significantly expressed in hMSCs. Transcripts of the following key genes of the neuronal dopaminergic system were detected in hMSCs and in neuronally-differentiated hMSCs by RT-PCR: Dopa decarboxylase, GTP cyclohydrolase 1, aromatic L-amino acid decarboxylase (AADC), catechol-O-methyltransferase (COMT) and the transcription factor Nurr-1.

Northern Blot Analysis:

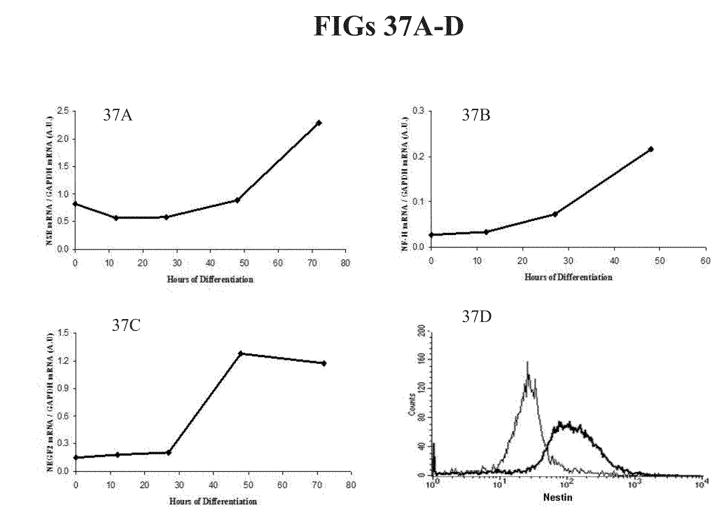

RNA levels of human NEGF2 (FIG. 37C), NF-H (FIG. 37B), and NSE (FIG. 37A) were assessed by Northern blot analysis, demonstrating an increase in the expression of all three transcripts following incubation in differentiation medium.

Western Blot Analysis:

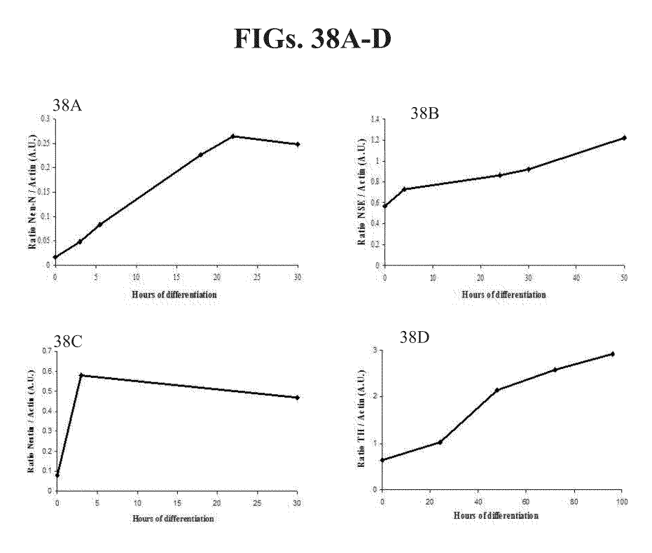

The presence of neural proteins in hMSCs was detected by Western blot. Expression of NSE, nestin and the oligodendrocyte CNPase proteins were found to be expressed by non-differentiated hMSCs (FIGS. 38A-C and 38E). Neu-N, NSE and nestin proteins were markedly increased following neuronal differentiation as measured in comparison with actin levels (FIGS. 39 A-C).

The tyrosine hydroxylase (TH) protein, which is the rate-limiting enzyme in the biosynthesis of catecholamines and is a marker of ventral midbrain neurons, was detected in MSCs, by Western blot analysis (FIG. 38D). Furthermore, protein levels were significantly elevated during the neuronal differentiation (FIG. 39D).

Flow Cytometry Analysis:

A vast majority (approximately 84.43%) of the undifferentiated hMSCs population express the nestin protein (FIG. 37D).

Protein Array Analysis:

Results from the protein array are detailed below in table 9. 39 out of 96 transcription factors were found to be significantly changed.

TABLE 9
Thirty nine transcription factors investigated by DNA protein array analysiswhich
were found to be significantly changed

| | | | Additional |
|---|---|---|---|
| Transcription factor | ODu * mm² | Reference | information |
| AFXH | 1.346409 | Biggs WH IIIrd | Member of the |
| | | et al 2001 | forkhead family. |
| | | | (Foxo4) Xq13 |
| Aryl hydrocarbon receptor/aryl hydrocarbon receptor nuclear | 1.094392 | Ema M, 1994, | 7p21 |
| translocator binding element. (AhR/Arnt) | | Fujii-Kuriyama | |
| | | Y, 1994 | |
| | | (TRANSFAC) | |
| Cardiac enhancer factor-1 (CEF-1) | 1.533479 | Parmacek MS, | region of the |
| | | 1992 | Slow/Cardiac |
| | | | Troponin C |
| | | | enhancer. |
| Cardiac enhancer factor-2 (CEF2) | 0.640472 | Parmacek MS, | region of the |
| | | 1992 | Slow/Cardiac |
| | | | Troponin C |
| | | | enhancer. |
| Cholesteryl ester transfer protein | 1.86282 | Saito K, 1999 | |
| (CETP/CRE) | | | |
| Ecotropic viral integration site 1 (EVI-1) | 1.070068 | Kim JH, 1998 | zinc finger oncogene |
| FKHR human | 3.523945 | Leenders H,. | human forkhead box |
| | | 2000 | O1A |
| | | | (rhabdomyosarcoma) |
| FKHR mouse | 2.01917 | Biggs WH IIIrd, | forkhead box O1 |
| | | 2001 | mouse |
| Fork head Related Activator-2 (Freac-2) | 3.419156 | Hellqvist M, | forkhead box F2 |
| | | 1996 | |
| | | (TRANSFAC) | |
| GAG | 2.451158 | Hoffman PW, | amyloid precursor |
| | | 1995 | protein (APP) |
| | | | regulatory element |
| Gamma-interferon activation site (GAS) | 0.691235 | Decker T, 1991 | |
| GATA binding globin transcription factor 1 (GATA-1) | 1.930978 | Newton A, 2001 | Globin gene in |
| | | | erythrocytes |
| GATA binding globin transcription factor 2 (GATA-2) | 1.55914 | Yamashita K, | Endothelial |
| | | 2001 | |
| GATA binding globin transcription factor 4 (GATA-4) | 2.078057 | Hung HL, 2001 | Regulation of IL-5 |
| Growth factor independent 1 (Gfi-1) | 3.526001 | Zweidler-Mckay | |
| | | PA, 1996 | |
| HIF-1 binding site (HBS) and its downstream HIF-1 ancillary | 3.243141 | Kimura H, 2001 | |
| sequence (HAS), combined. | | | |
| HIF-1 binding site/rat, as human xbp-1 (HBS/xbp-1) | 3.16012 | Gene 2000, | |
| | | 241: 297-307. | |
| Hepatocyte nuclear factor 3 beta (HNF-3b) | 1.565933 | Eur. J. Immunol. | (forkhead domain) |
| | | 2000, 30: 2980-2991 | |
| Interferon regulatory factor ½ binding element (IRF-1/2) | 2.090715 | (TRANSFAC) | |
| Interferon-a stimulated response element (ISRE-1) | 1.960459 | EMBO (1995, | |
| | | 14: 1166-1175) | |
| Pyruvate kinase L gene binding element III (L-III BP) | 0.733756 | BBRC 1999, | (hepatocyte |
| | | 257: 44-49. | specific) |
| Myelin gene expression factor 2 (MEF-2(2)) | 2.327848 | EMBO J. 1994, | |
| | | 13: 3580-3589. | |
| Myelin gene expression factor 3 (MEF-3) | 1.777975 | Mol Cell Biol. | |
| | | 1992 | |
| | | May; 12(5): 1967-76. | |
| MSP-1 | 1.633083 | Nucleic Acids | the sequences are the |
| | | Research 1995, | same as SAA except |
| | | 23: 2229-2235. | SP1 binding site is |
| | | | removed |
| Myeloid-specific retinoic acid-responsive zinc finger protein | 3.233982 | Curr Top | |
| (MZF1) | | Microbiol | |
| | | Immunol. | |
| | | 1996; 211: 159-64. | |
| | | Review | |
| Nuclear Y box factor (NF-Y) | 1.603387 | (TRANSFAC) | |
| Neural zinc finger factor 3 (NZF-3) | 0.808427 | JBC 1998, | |
| | | 273: 5366-5374. | |
| Poly(ADP-ribose) synthetase/polymerase (PARP) | 1.148752 | PNAS 2001 | |
| | | 98: 48-53 | |
| Paired box gene 3 (Pax-3) | 2.331622 | JBC 1997, | |
| | | 272: 14175-14182. | |
| Paired box gene 4 (Pax-4) | 1.790419 | Mol. Cell. Biol. | |
| | | 1999, 19: 8272-8280. | |
| Paired box gene 6 (Pax6) | 0.920277 | Gene 2000, | |
| | | 245: 319-328. | |
| Paired box gene 8 (Pax-8) | 1.202082 | JBC, 1997, | |
| | | 272: 30678-30687. | |
| Ras-responsive transcription element (RREB-1) | 0.854537 | Nucleic Acids | |
| | | Research 1999, | |
| | | 27: 2947-2956. | |
| Ras-responsive transcription element (RREB-2) | 2.710857 | Mol. Cell. Biol. | |
| | | 1996, 16: 5335-5345. | |
| Related to serum response factor, C4 (RSRFC4) | 3.75185 | (TRANSFAC) | MADS box |
| | | | transcription |
| | | | enhancer factor 2 |
| SAA | 0.656215 | NAR (1995, | amyloid precursor |
| | | 23: 2229-2235) | protein (APP) |
| | | | regulatory element |
| | | | containing potential |
| | | | binding site for SP1, |
| | | | AP4, USF, AP1 |
| Skn | 2.164018 | JBC 1997, | octamer-binding site |
| | | 272: 15905-15913. | in epidermis (POU |
| | | | domain factor) |
| X3enobiotic response element (XRE) | 1.277671 | BBRC 1999, | |
| | | 256: 133-137. | |
| ZIC | 0.817573 | J. Virol. 1996, | one of four DNA |
| | | 70: 3894-3901. | binding domains on |
| | | | BZLF1 gene (EBV |
| | | | virus) promotor |

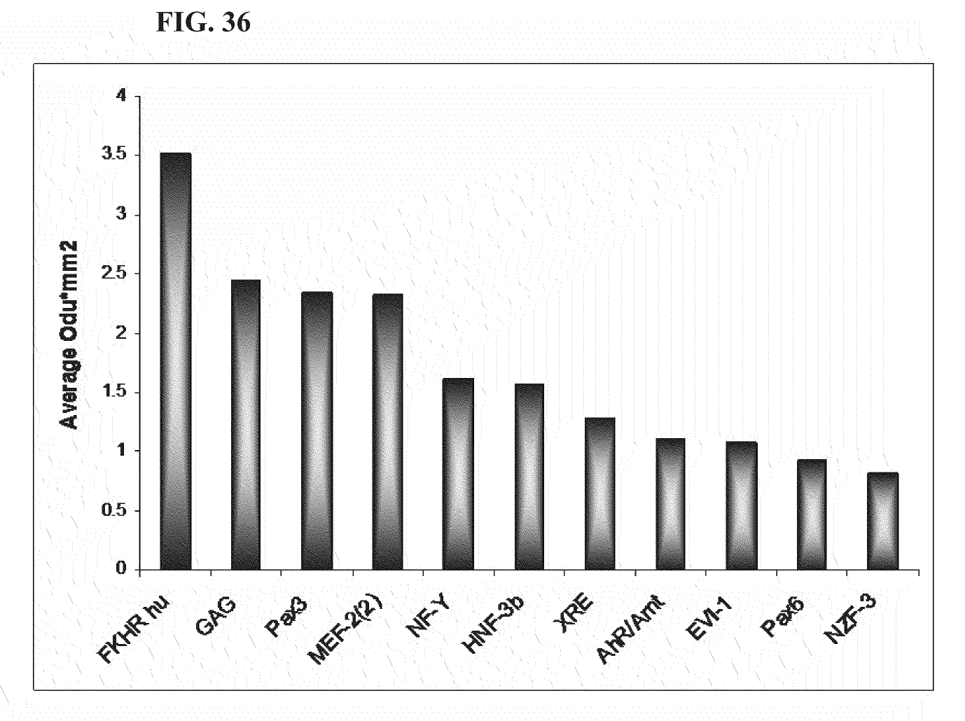

A total of 39 transcription factors were found to be active in hMSCs (Table 9). Of the 39, 11 transcription factors have an established neuronal involvement (i.e. have a known role in neural function, differentiation and gene expression and are expressed in the CNS; FIG. 36). The 11 transcription factors with neural involvement include AhR/ARNT, EVI-1, FKHRhu, GAG, HNF-3β, MEF-2(2), NF-Y, Pax-3, Pax-6, NZF-3, and XRE. After 24 hours of neuronal differentiation the cells showed an increase in the level of NZF-3, and a significant decrease in the levels of GAG, EV-1, FKHRhu, HNF-3B and XRE, the rest of the transcription-factors remaining unchanged, compared to control untreated MSCs. However, following 48 hours of neuronal differentiation, a significant elevation was observed in the levels of the neural transcription factors GAG, EVI-1 and FKHRhu, while there was a significant decrease in the levels of NF-Y, Pax-3, Pax-6, NZF-3, MEF-2(2) and AhR/ARNT. No significant change was found in the levels of HNF-3β or XRE, following the 48 h neuronal differentiation. Thus a significant fraction of the active transcription factors expressed by hMSCs are factors with a documented neural involvement, which are affected by neuronal differentiation, suggesting a role in the process.

Conclusions

The neural gene expression in naïve hMSCs suggests that they posses a potential for plasticity and differentiation to neural derivatives. Results from FACS analysis, Western blot studies, RT-PCR studies and immunohistochemistry studies indicate that a significant fraction of the undifferentiated hMSCs express neural genes. For some of these genes, this expression is enhanced when the stem cells undergo differentiation to neuronal phenotype.

From these results, it may be suggested that the genes expressed by undifferentiated stem cells, such as neural genes, make the cells prone to mature to neural phenotypes enabled by these genes, rather than to phenotypes of which the cell does not yet expresses any genes. A neighboring cell, that doesn't express any neural genes would, by this logic, encounter more difficulty in differentiation to a neural phenotype.

### Example 21

### Improvement of Functioning of Neuronally Differentiated BMSc by the Alteration of Fatty Acid Composition in the Additional Differentiation Medium

Polyunsaturated essential fatty acids (PUFA) are necessary for intact neuronal functioning. The improvement of the function of neuronally differentiated BMSc was analyzed by altering the fatty acid composition in the predifferentiation medium.

Methods:

Isolation and Incubation of Human BMSCs:

Human BMSCs were isolated as described in Examples 1 and 19 and incubated in the proliferation medium detailed below.

Proliferation Medium:

Dulbecco's modified eagle medium (DMEM; Biological Indutries) 100 μg/ml streptomycin, 100 U/ml penicillin, 12.5 units/ml nystatin (SPN; Biological Industries); 2 mM L-glutamine; 15% fetal calf serum (FCS; Biological Industries); ±0.001% 2-β-mercaptoethanol (Sigma); ±1× non-essential amino acids; ±10 ng/ml human epidermal growth factor (EGF). Growth medium was changed twice a week and cells were maintained at 37° C. in a humidified 5% CO₂ incubator in normal or in low oxygen (O₂-3%, N₂-72%). The nonadherent cells were removed during medium replacement, leaving the tightly adhered BMSC.

Neuronal Differentiation:

The growth medium was replaced with the “additional differentiation medium” for 48 hr as described in table 1 at Example 2 with increasing concentrations of 40 μM docosahexaenoic acid (DHA; Sigma) and 40 μM arachidonic acid (AA; Sigma) alone and in combination. α-Tocopherol (40 μM, Sigma) was included in all cultures, including the control, to prevent fatty acid oxidation. DHA and AA were diluted in DMEM with 1% horse serum. α-tocopherol was dissolved in ethanol (absolute) before supplementation. FA and α-tocopherol were stored at 4° C. in darkness to prevent their oxidation. Following forty-eight hours, the “additional differentiation medium” was changed to “differentiation medium”, containing DMEM supplemented with SPN, 2 mM L-glutamine, 200 μM butylated hydroxyanisole (BHA), 1 mM dibutyryl cyclic AMP (dbcAMP), 0.5 mM isobutylmethylxanthine (IBMX), 1 μM retinoic acid (RA) and N2 supplement with increasing amounts of DHA (Sigma) and AA (Sigma) (0, 30, 40, 50 and 60 μM). α-tocopherol was added at a concentration of 40 μM to all samples.

Immunostaining:

Cells were fixed with 4% paraformaldehyde in PBS, for 30 minutes. Following permeabilization with 0.5% Triton in PBS, cells were blocked for 1 hour with PBS containing 10% goat serum and 2% immunoglobulin free BSA, and then incubated with the primary antibodies against MAP-2ab (mouse monoclonal 1:300). Following washing with PBS, cells were incubated with the corresponding Cy3 (1:800). Dapi nuclear dye was applied according to manufacturer instructions.

Neurite Measurement:

To study the effect of FA on the morphology of dendrites, cells were stained with anti-MAP2 antibody to reveal the somatodendritic compartment after induced neuronal differentiation. To minimize bias, neurons were blindly traced. Fields were chosen at random and only non-clustered neuron-like cells were traced to ensure the precision of the measurements. The neurite length was determined with the ImagePro software and 20 neurons were measured in every case. The total neurite length of neuron-like cells was determined by measuring the individual neurite-like extensions lengths with the ImagePro Software and summing them per neuron.

Western Blot Analysis:

Following induced neuronal differentiation, BMSC were homogenized in lysis buffer containing 50 mM Tris-HCl, pH 7.5, 150 mM NaCl, 2 mM EDTA, 1% NP-40, 5 μg/ml aprotinin, 5 μg/ml leupeptin, and 17 μg/ml PMSF. The homogenate was centrifuged at 14,000×g at 4° C., and the protein concentration of the supernatant was determined with a micro BCA kit (Pierce, Rockford, Ill.). The proteins (25 μg protein) were denatured in 1:5 sample buffer (62.5 mM Tris-HCl, pH 6.8, 10% Glycerol, 2% sodium dodecyl sulfate, 5% 2-β-mercaptoethanol, 0.0025% bromophenol blue). Each sample was loaded onto 12.5% SDS-polyacrylamide gel (ratio acrylamide/bis-acrylamide 37.5/1) (Bio-Rad Laboratories, Hercules, Calif., USA) according to the manufacturer's instructions and transferred to nitrocellulose membranes. A blocking solution of 5% nonfat milk in Tris-buffered saline with Tween 20 (TBS-T; 10 mM Tris, pH 7.5, 150 mM NaCl, 0.05% Teen 20) was applied. Blots were probed at room temperature for two hours with the mouse anti-TH (diluted at 1:10,000 in 5% nonfat milk in TBS-T) or mouse anti-synaptophysin (diluted at 1:1000 in 5% nonfat milk in TBS-T). Equal loadings of proteins were probed with mouse monoclonal antibody to actin (Chemicon, Temecula, Calif., USA) diluted at 1:10,000 in 5% nonfat milk in TBS-T. Membranes were washed with 5% nonfat milk TBS-T three times (5 min each) and then exposed to horseradish-peroxidase conjugated goat anti-mouse IgG (Jackson ImmunoResearch Laboratories, West Grove, Pa., USA) diluted 1:20000 in 5% nonfat milk TBS-T, for 1 hour at room temperature and washed twice with 5% nonfat milk TBS-T and once with TBS-T (5 min each). Proteins of interest were detected using the enhanced SuperSignal® chemiluminescent Western blotting detection system kit (Pierce, USA). Blots were incubated in working solution for 2 minutes, and exposed to medical X-ray film (Fuji Photo Film, Tokyo, Japan). X-ray film densitometry was performed using Versa Doc® imaging system (BIO RAD Laboratories Hercules, Calif., USA).

Lipid Extraction:

Following removal of medium from the culture dish, adherent cells were washed twice with cold PBS and dried by gentle aspiration with a Pasteur pipette. To each culture dish, 3 ml of a hexane:isopropanol (3:2 vol:vol) (HIP) solvent mixture containing 5 mg/dl butylated hydroxytoluene (BHT) was added. The dishes were gently shaken at room temperature for 10 minutes and the supernates were transferred to methylation tubes. The procedure was repeated with 1.5 ml HIP and the lipid extracts were added to the same tubes. For quantitative experiments which included simultaneous protein determination, lipid extraction was performed with HIP which contained a known amount of heneicosanoic acid (21:0) so as to obtain an internal standard. In addition, for these experiments, known volumes of concentrated cell suspensions were directly extracted with HIP and the protein content was determined from an equal volume of cell concentrates.

Fatty Acid Methylation:

The lipid extracts were evaporated to dryness under a stream of nitrogen. To each tube, 1 ml of 14% BF₃ in methanol and 0.5 ml of benzene were added. The tubes were capped under N₂, vortexed and heated at 100° C. for 1 hour. The resulting fatty acid methyl esters (FAME) were extracted into hexane, after acidification of the mixture with 3 drops of 3N HCl, and kept under N₂ at −20° C. until analysis.

Gas Chromatography (GC):

FAME separation was performed on an HP 5890 Series II gas chromatograph, utilizing a polar capillary column (SGE), 30 m×0.25 mm ID×0.25 microns film thickness, and a flame ionization detector. Injection was performed with a split/splitless injector kept at 220° C. at a split ratio of 1:30. Detector temperature was 250° C. The column temperature gradient was as follows: 70° C. for 2 min, increased at a rate of 20° C./min to 150° C. where it remained for 5 min, increased at 3° C./min to 210° C. and a final increase at 10° C./min to 230° C. at which it was kept for 3 min until the end of the run. FAME peaks were integrated and computed with the aid of the Varian Star Integrator computer package and were identified by comparison of their retention times with that of authentic standards. Injection of the samples was performed with the aid of a CTC-AS 200 autosampler.

Fatty Acid Data Analysis:

The amount of individual fatty acids is presented as % weight of the total identified fatty acids and as μg FA/μg protein.

Results

The FA composition of cells grown in the absence of added PUFA to the medium displayed a profile characteristic of peripheral non-neuronal type cells (Table 10, −DHA) and completely unlike normal neural tissue (for example, in normal rat striatum tissue, DHA constitutes 13.09±1.44% weight and AA 10.28±0.52% weight [Green et al., 2005]). Supplementation of the additional differentiation medium with DHA (30 μM) resulted in cellular PUFA composition approaching that of normal neural tissue Table 9, +DHA). FA analysis showed that DHA content (% weight) increased continuously with increasing DHA concentration. However, it was also observed that AA concentration decreased with increasing DHA concentrations, suggesting that AA suplementation might also be required. The optimal concentration of DHA and AA required in the additional differentiation medium to obtain the best cellular FA profile was found to be 40 μM for both fatty acids.

TABLE 10
Effect of DHA supplementation in the additional differentiation and
differentiation medium on the fatty acid composition of BMSC

| | |
|---|---|
| Additional | |
| differentiation | Differentiation |

| Fatty acid* | −DHA | +DHA | −DHA | +DHA |
|---|---|---|---|---|
| Sum Saturated | 41.35 | 42.69 | 45.71 | 61.79 |
| Sum MUFA | 34.48 | 24.24 | 29.74 | 12.32 |
| 20:4 n-6 | 9.10 | 8.15 | 9.19 | 5.40 |
| 22:6 n-3 | 4.93 | 16.70 | 5.07 | 13.01 |
| Sum PUFA | 24.21 | 33.08 | 24.44 | 25.71 |

*Saturated fatty acids include 14:0, 16:0, 18:0, 20:0, 22:0, 24:0. Monounsaturated fatty acids (MUFA) include 16:1, 18:1, 20:1, 24:1. Saturated FA and MUFA are not essential FA. PUFA include, in addition to AA and DHA, also their precursors (18:2n-6, 18:3n-3, 20:3n-6, 20:3n-3, 20:5n-3, 22:5n-3) and their longer-chain metabolites (22:4n-6, 22:5n-6).

α-Tocopherol, 40 μM, did not affect the PUFA composition of the neuron-like cells, and was added as an antioxidant in all experiments.

Following DHA, AA and α-Tocopherol addition to the additional differentiation medium of human BMSc undergoing induced neural differentiation, quantitative analysis of FA content (FA content/protein amount) indicated that the amount of the overall PUFA was increased. In addition, DHA content of the neuron-like cells was increased, with no loss of AA observed. To evaluate if FA supplementation in vitro enhanced the neurite growth of the neuron-like cells, the length of neurite-like extensions following induced neuronal differentiation of BMSC was examined. As seen in FIG. 39 and FIGS. 40A and 40B, cultures treated with FA during induced neuronal differentiation showed an increase in the population of neurons with longer total neurite lengths (100-200 μm and higher) and a decrease in the number of neurons with shorter total neurite lengths (0-100 μm ranges). Moreover, the sum of total neurite lengths in 20 neuron-like cells was 1432 μm for control which increased to 2291 μm following PUFA supplementation. The observed difference in the total neurite length per neuron from control and PUFA-treated cells was statistically significant when compared by student t-test.

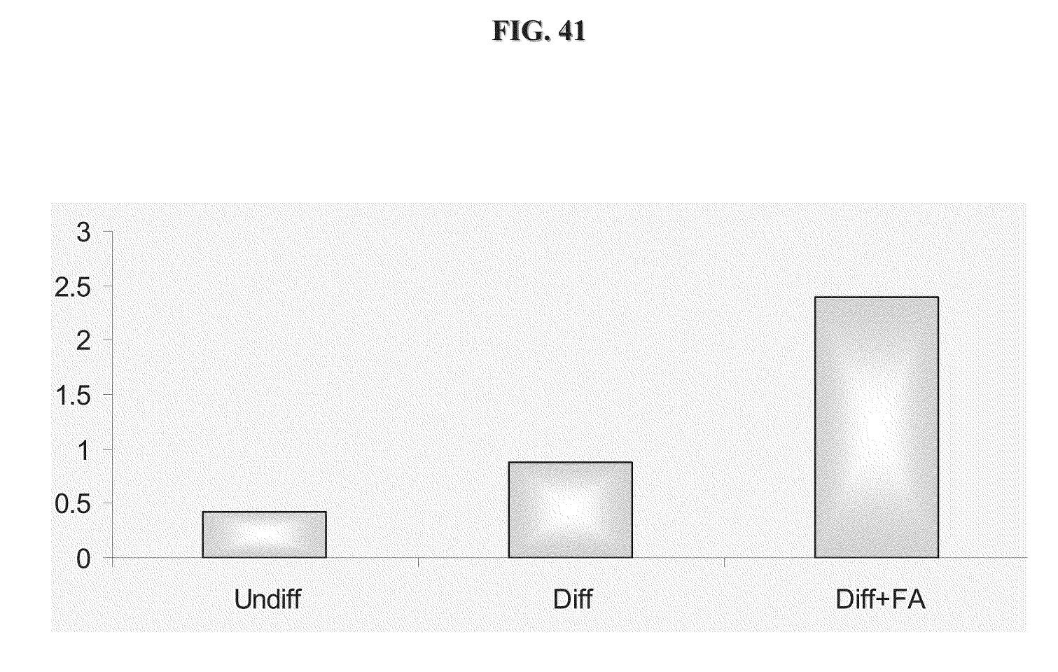

As illustrated in FIG. 41, PUFA supplementation increased the expression of synaptophysin. No effect on TH expression was observed.

In conclusion, addition of PUFA to the predifferentiation media of BMSC contributed to the intact functioning of the differentiated cells.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications and GenBank accession numbers mentioned in this specification are herein incorporated in their entirety by reference into the specification, to the same extent as if each individual publication, patent or patent application or GenBank accession number was specifically and individually indicated to be incorporated herein by reference. In addition, citation or identification of any reference in this application shall not be construed as an admission that such reference is available as prior art to the present invention.

### REFERENCES CITED

### Additional References are Cited Hereinabove

Aguirre T, Van Den Bosch L, Goetschalckx K, Tilkin P, Mathijs G, Cassiman J J, Robberecht W. 1998. Increased sensitivity of fibroblasts from amyotrophic lateral sclerosis patients to oxidative stress. Ann Neurol 43:452-457.
Azizi S A, Stokes D, Augelli B J, DiGirolamo C, Prockop D J. 1998. Engraftment and migration of human bone marrow stromal cells implanted in the brains of albino rats: similarities to astrocyte grafts. Proc Natl Acad Sci USA 95:3908-3913.
Bakay R A E, Fiandaca M S, Barrow D L, Schiff A, Collins D C. 1985. Preliminary report of the use of fetal tissue transplantation to correct MPTP-induced Parkinson-like syndrome in primates. Appl Neurophysiol 48:358-361.
Bankiewicz K S, Plunkett R J, Jacobowitz D M, Porrino L, Porzio U D, London W T, Kopin U, Oldfield E H. 1990. The effect of fetal mesencephalon implants on primate MPTP-induced parkinsonism: Histochemical and behavioral studies. J Neurosurg 72:231-244.
Bendotti C, Tortarolo M, Suchak S K, Calvaresi N, Carvelli L, Bastone A, Rizzi M, Rattray M, Mennini T. 2001. Transgenic SOD1 G93A mice develop reduced GLT-1 in spinal cord without alterations in cerebrospinal fluid glutamate levels. J Neurochem 79:737-746.
Ben-Hur, T., Idelson, M., Khaner, H., Pera, M., Reinhartz, E., Itzik, A., Reubinoff, B. E. 2004 Transplantation of human embryonic stem cell-derived neural progenitors improves behavioral deficit in Parkinsonian rats. Stem Cells. 22: 1246-1255.
Bjorklund A, Stenevi U. 1979. Reconstruction of the nigrostriatal dopamine pathway by intracerebral nigral transplants. Brain Res 177:555-560.
Bjorklund L M, Sanchez-Pernaute R, Chung S, Andersson T, Chen I Y, McNaught K S, Brownell A L, Jenkins B G, Wahlestedt C, Kim K S, Isacson O. 2002. Embryonic stem cells develop into functional dopaminergic neurons after transplantation in a Parkinson rat model. Proc Natl Acad Sci USA 99:2344-2349.
Bottenstein J. E. (1985) Growth of neural cells in defined media, in Cell Culture in the Neurosciences, Bottenstein, J. E. and Sato G., Plenum Press, New York, N.Y., PP. 1-40.
Brazelton T R, Rossi F M V, Keshet G I, Blau H M. 2000. From marrow to brain: expression of neuronal phenotypes in adult mice. Science 290:1775-1779.
Breeze R, Wells T H, Freed C R. 1995. Implantation of fetal tissue for the management of Parkinson's disease: A technical note. Neurosurgery 36:1044-1047.
Brundin P, Karlsson J, Emgard M, Kaminski-Schierle G S, Hnssson O, Petersen A, Castilho R F. 2000. Improving the survival of grafted dopaminergic neurons: a review over current approaches. Cell Transplant 9:179-195.
Chen J, Li Y, Wang L, Zhang Z, Lu D, Lu M, Chopp M. 2001. Therapeutic benefit of intravenous administration of bone marrow stromal cells after cerebral ischemia in rats. Stroke 32: 1005-1011.
Choi-Lundberg D L, Lin Q, Schallert T, Crippens D, Davidson B L, Chang Y N, Chiang Y L, Qian J, Bardwaj L, Bohn M C. 1998. Behavioral and cellular protection of rat dopaminergic neurons by an adenoviral vector encoding glial cell line-derived neurotrophic factor. Science 154:261-275.
Chomczynski P, Sacchi N. 1987. Single-step method of RNA isolation by acid guanidinium thiocyanate-phenol-chloroform extraction. Anal Biochem 162:156-159.
Chopp M, Zhang X H, Li Y, Wang L, Chen J, Lu D, Lu M, Rosenblum M. 2000. Spinal cord injury in rat: treatment with bone marrow stromal cell transplantation. Neuroreport 11: 3001-3005.
Corti O, Sabate O, Horellou P, Colin P, Dumas S, Buchet D, Buc-Caron M H, Mallet J. 1999a. A single adenovirus vector mediates doxycycline-controlled expression of tyrosine hydroxylase in brain grafts of human neural progenitors. Nat Biotech 17:349-354.
Corti O, Sanchez-Capelo A, Colin P, Hanoun N, Hamon M, Mallet J. 1999b. Long-term doxycycline-controlled expression of human tyrosine hydroxylase after direct adenovirus-mediated gene transfer to a rat model of Parkinson's disease. Proc Natl Acad Sci USA 96:12120-12125.
Costantini L C, Bakowska, J C, Breakefild X O, Isacson O. 2000. Gene therapy in the CNS. Gene Therapy 7:93-109.
Cudkowicz M E, McKenna-Yasek D, Sapp P E, Chin W, Geller B, Hayden D L, Schoenfeld D A, Hosler B A, Horvitz H R, Brown R H. 1997. Epidemiology of mutations in superoxide dismutase in amyptrophic lateral sclerosis. Ann Neurol 41: 210-221.
Deng W, Obrocka M, Fischer I, Prockop D J. 2001. In vitro differentiation of human marrow stromal cells into early progenitors of neural cells by conditions that increase intracellular cyclic AMP. Biochem Biophys Res Commun 282:148-152.
Dunnett S B, Annett L E. 1991. Nigral transplants in primate models of parkinsonism, in Lindvall O, Bjorklund A, Widner H (eds) Restorative Neurology: Intracerebral Transplantation in Movement Disorders. Amsterdam, Elsevier, vol 4, pp 27-51.
Fallon J, Reid S, Kinyamu R, Opole I, Opole R, Baratta J, Korc M, Endo T L, Duong A, Nguyen G, Karkehabadhi M, Twardzik D, Patel S, Loughlin S (2000) In vivo induction of massive proliferation, directed migration, and differentiation of neural cells in the adult mammalian brain. Proc Natl Acad Sci USA. 97: 14686-14691)
Freed C R, Breeze R E, Rosenberg N L, Schneck S A, Kriek E, Qi J-X, Lone T, Zhang Y-B, Snyder J A, Wells T H, Ramig L O, Thompson L, Mazziotta J C, Huang S C, Grafton S T, Brooks D, Sawle G, Schroter G, Ansari A A. 1992. Survival of implanted fetal dopamine cells and neurologic improvement 12 to 46 months after transplantation for Parkinson's disease. N Engl J Med 317:1549-1555.
Freed C R, Breeze R E, Rosenberg N L, Schneck S A, Wells T H, Barrett J N, Grafton S T, Huang S C, Eidelberg D, Rottenberg D A. 1990. Transplantation of human fetal dopamine cells for Parkinson's disease: Results at 1 year. Arch Neurol 47:505-512.
Freed C R, Breeze R E, Rosenberg N L, Schneck S A. 1993. Embryonic dopamine cell implants as a treatment for the second phase of Parkinson's disease: Replacing failed nerve terminals, in Narabayashi H, Nagatsu T, Yanagisawa N, Mizuno Y (eds). Advances in Neurology. New York, Raven Press, Ltd, vol 60, pp. 721-728.
Freed C R, Greene P E, Breeze R E, Tsai W Y, DuMouchel W, Kao R, Dillon S, Winfield H, Culver S, Trojanowski J Q, Eidelberg D, Fahn S. 2001. Transplantation of embryonic dopamine neurons for severe Parkinson's disease. N Engl J Med 344:710-719.
Freed C R, Richards J B, Sabol K E, Reite M. 1988. Fetal substantia nigra transplants lead to dopamine cell replacement and behavioral improvement in Bonnet monkeys with MPTP induced parkinsonism, in Beart P M, Woodruff G, Jackson D M (eds), Pharmacology and Functional Regulation of Dopaminergic Neurons. London, Macmillan Press, pp 353-363.
Freeman T B, Olanow C W, Hauser R A, Nauert G M, Smith D A, Borlongan C V, Sanberg P R, Holt D A, Kordower J H, Vingerhoets F J, et al. 1995. Bilateral fetal nigral transplantation into the postcommissural putamen in Parkinson's disease. Ann Neurol 38:379-388.
Garcia M., Ward G., Ma Y.-C., Salem N., Kim H.-Y. 1998. Effect of docosahexaenoic acid on the synthesis of phosphatidylserine in rat brain microsomes and C6 glioma cells. J Neurochem 70:24-30.
Green P, Yavin E. 1995. Modulation of fetal rat brain and liver phospholipid content by intaamniotic ethyl docosahexaenoate administration. J Neurochem 65:2555-2560.
Green P, Gispan-Herman I, Yadid G. 2005. Increased arachidonic acid concentration in the brain of Flinders Sensitive Line rats, an animal model of depression. J Lipid Res, April 1, doi:10.1194/jlr.C500003-JLR200.
Grunblatt E, Mande S, 2000 Neuroprotective Strategies in Parkinson's Disease Using the Models of 6-Hydroxydopamine and MPTP^{a} Annals of the New York Academy of Sciences 899:262-273.
Guegan C, Przedborski S. 2003. Programmed cell death in amyotrophic lateral sclerosis. J Clin Invest. 111:153-161.
Guégan C, Vila M, Rosoklija G, Hays A P, and Przedborski S. 2001. Recruitment of the Mitochondrial-Dependent Apoptotic Pathway in Amyotrophic Lateral Sclerosis. The J. Neurosci, 21:6569-6576.
Gurney M E, Cutting F B, Zhai P, Doble A, Taylor C P, Andrus P K, Hall E D. 1996. Benefit of vitamin E, riluzole, and gabapentin in a transgenic model of familial amyotrophic lateral sclerosis. Ann Neurol 39:147-157.
Gurney M E, Pu H, Chiu A Y, Dal Canto M C, Polchow C Y, Alexander D D, Caliendo J, Hentati A, Kwon Y W, Deng H X, et al. 1994. Motor neuron degeneration in mice that express a human Cu, Zn superoxide dismutase mutation. Science 264:1772-1775.
Hadjantonakis A K, Gertsenstein M, Ikawa M, Okabe M, Nagy A. 1998. Generating green fluorescent mice by germline transmission of green fluorescent ES cells. Mech Dev 76:79-90.
Heath P R, Shaw P J. 2002. Update on the glutamatergic neurotransmitter system and the role of excitotoxicity in amyotrophic lateral sclerosis. Muscle Nerve 26:438-458. Hill W D, Hess D C, Martin-Studdard A, Carothers J J, Zheng J, Hale D, Maeda M, Fagan S C, Carroll J E, Conway S J. 2004. SDF-1 (CXCL12) is upregulated in the ischemic penumbra following stroke: association with bone marrow cell homing to injury. J Neuropathol Exp Neurol. January; 63(1):84-96.
Holiday R. 1996. DNA methylation in eukaryotes: 20 years on, in Russo V E A, Martienssen R A, Riggs A D, Epigenetic mechanisms of gene regulation. New York: Cold Spring Harbor Laboratory Press. pp 692.
Hwan-Wun L, Ya-Jane K, Jiahn-Chun W, Kuo-Hsing M, Shwun-De W, Jiang-Chuan L. 1999. Intrastriatal transplantation is Sertoli cells may improve amphetamine-induced rotation and tyrosine hydroxylase immunoreactivity of the striatum in hemiparkinsonian rats. Brain Res 838:227-233.
Ischiropoulos H, Beckman J S. 2003. Oxidative stress and nitration in neurodegeneration: cause, effect, or association? J Clin Invest. 11:163-169.
Jackson-Lewis V, Liberatore G. 2000. Effects of a unilateral stereotaxic injection of Tinuvin 123 into the substantia nigra on the nigrostriatal dopaminergic pathway in the rat. Brain Res. June 2; 866(1-2):197-210.
Kan, E. Melamed, D. Offen, (2005) Integral therapeutic potential of bone marrow mesechymal stem cells. Current Drug Targets, 6.
Kim H-Y., Akbar M., Lau A., Edsall L. 2000. Inhibition of neuronal apoptosis by docosahexaenoic acid (22:6n-3). J Biol Chem 275:35215-35223.
Kim, J. H., Auerbach, J. M, Rodríguez-Gómez, J. A., Velasco, I, Gavin, D., Lumelsky, N., Lee, S. H., Nguyen, J., Sanchez-Pernaute, R., Bankiewicz, K., McKay, R. 2002 Dopamine neurons derived from embryonic stem cells function in an animal model of Parkinson's disease. Nature 418: 50-56.
Kohyama J, Abe H, Shimazaki T, Koizumi A, Nakashima K, Gojo S, Taga T, Okano H, Hata J, Umezawa A. 2001. Brain from bone: efficient “meta-differentiation” of marrow stroma-derived mature osteoblasts to neurons with Noggin or a demethylating agent. Differentiation 68: 235-244.
Kopen G C, Prockop D J, Phinney D G. 1999. Marrow stromal cells migrate throughout forebrain and cerebellum, and the differentiate into astrocytes after injection into neonatal mouse brains. Proc Natl Acad USA 96:10711-10716.
Kordower J H, Freeman T B, Chen E Y, Mufson E J, Sanberg P R, Hauser R A, Snow B, Olanow C W. 1998. Fetal nigral grafts survive and mediate clinical benefit in a patient with Parkinson's disease. Movt Dis 13:383-393.
Kordower J H, Freeman T B, Snow B J, Vingerhoets F J G, Mufson E J, Sanberg P R, Hauser R., Smith D A, Nauert G M, Perl D P, Olanow C W. 1995. Neuropathological evidence of graft survival and striatal reinnervation after the transplantation of fetal mesencephalic tissue in patient with Parkinson's disease. N Engl J Med 332:1118-1124.
Lee, S. H., Lumelsky, N., Studer, L., Auerbach, J. M., McKay, R. D. 2000 Efficient generation of midbrain and hindbrain neurons from mouse embryonic stem cells. Nat Biotechnol. 18: 675-679.
Levy Y. S., Stroomza M, E. Melamed, D. Offen (2004) Embryonic and Adult Stem Cells as a Source for Therapy in Parkinson's Disease. J Mol. Neurosci. 24:353-386.
Levy Y S, Merims D, Panet H, Barhum Y, Melamed E, Offen D. (2003)
Induction of neuron-specific enolase promoter and neuronal markers in differentiated mouse bone marrow stromal cells. J Mol. Neurosci. 21:127-138.
Li Y, Chen J, Wang L, Zhang L, Lu M, Chopp M. 2001. Intracerebral transplantation of bone marrow stromal cells in a 1-methyl-4-phenyl-1,2,3,6-tetrahydropyridine mouse model of Parkinson's disease. Neurosci Lett 316:67-70.
Li Y, Chopp M, Chen J, Wang L, Gautam S C, Xu Y X, Zhang Z. 2000. Intrastriatal transplantation of bone marrow nonhematopoietic cells improves functional recovery after stroke in adult mice. J Cereb Blood Flow Metab 20:1311-1319.
Linder M D, Winn S R, Baetge E E, Hammang J P, Gentile F T, Doheety G E, McDermott P E, Frydel B, Ullman D, Schallert T, Emerich D F. 1995. Implantation of encapsulated catecholamines and GDNF-producing cells in rats with unilateral dopamine depletions and Parkinsonian symptoms. Exp Neurol 132:62-76.
Lindvall O, Brundin P, Widner H, Rehncrona S, Gustavii B, Frackowiak R, Leenders K L, Sawle G, Rothwell J C, Marsden C D, Bjorklund A. 1990. Grafts of fetal dopamine neurons survive and improve motor function in Parkinson's disease. Science 247:574-577.
Lindvall O. Prospects of transplantation in human neurodegenerative diseases. 1991. Trends Neurosci 14:376-384.
Mahmood A, Lu D, Wang L, Li Y, Lu M, Chopp M. 2001a. Treatment of traumatic brain injury in female rats with intravenous administration of bone marrow stromal cells. Neurosurgery 49:1196-1204.
Mahmood A, Lu D, Yi L, Chen J L, Chopp M. 2001b. Intracranial bone marrow transplantation after traumatic brain injury improving functional outcome in adult rats. J Neurosurg 94:589-595.
Martin L J, Price A C, Kaiser A, Shaikh A Y, Liu Z. 2000. Mechanisms for neuronal degeneration in amyotrophic lateral sclerosis and in models of motor neuron death. Int J Mol Med 5:3-13.
McGeer P. L. and McGeer E. G. 2002 Inflammatory processes in amyotrophic lateral sclerosis Muscle Nerve 26:459-470.
McGeer, P. L. et al. 1988 Expression of the histocompatibility glycoprotein HLA-DR in neurological disease. Acta Neuropathol. 76: 550-557.
McKay R. 1997. Stem cells in the nervous system. Science 276: 66-71.
Mezey E, Chandross K J, Harta G, Maki R A, McKercher S R. 2000. Turning blood into brain: cells bearing neuronal antigens generated in vivo from bone marrow. Science 290:1779-1782.
O'Malley K L, Anhalt M J, Martin B M, Kelsoe J R, Winfield S L, Ginns E I. 1987. Isolation and charcterization of the human tyrosine hydroxylase gene: Identification of 5′ alternative splice sites responsible for multiple mRNAs. Biochemistry 26:6910-6914.
Olanow C W, Goetz C G, Kordower J H, Stoessl A J, Sossi V, Brin M F, Shannon K M, Nauert G M, Perl D P, Godbold J, Freeman T B, 2003. A double-blind controlled trial of bilateral fetal nigral transplantation in Parkinson's disease. Ann Neurol. 54:403-14.
Offen D, Sherki Y, Melamed E, Fridkin M, Brenneman D E, Gozes I. 2000. Vasoactive intestinal peptide (VIP) prevents neurotoxicity in neural cultures: relevance to neuroprotection in Parkinson's disease. Brain Res 854:257-262.
Pasinelli P., et al 1. 2000. Caspase 1 and 3 are sequentially activated in motor neuron death in Cu, Zn superoxide dismutase-mediated familial amyotorophic lateral sclerosis. Proc. Natl. Acad. Sci. USA 97:13901-13906/
Patridge T A, Davies K E. 1995. Myoblast-based gene therapies. Br Med Bull 51:123-137.
Paxinos G, Watson C. 1986. The rat brain sterotaxic coordinate. New York Academic.
Paxinos G, Franklin K. B. J. 2001. The Mouse Brain in Stereotaxic Coordinates, Second Edition, New York, Elsevier.
Peng H, Huang Y, Rose J, Erichsen D, Herek S, Fujii N, Tamamura H, Zheng J. 2004. Stromal cell-derived factor 1-mediated CXCR4 signaling in rat and human cortical neural progenitor cells. J Neurosci Res. April 1; 76(1):35-50
Perese D A, Ulman J, Viola J, Ewing S E, Bankiewicz K S. 1989. A 6-hydroxydopamine-induced selective parkinsonian rat model. Brain Res 494:285-93.
Perry V H, Gordon S. 1998. Macrophages and microglia in the nervous system. Trends Neurosci 11:273-277.
Piccini P, Brooks D J, Bjorklund A, Gunn R N, Grasby P M, Rimoldi O, Brundin P, Hagell P, Rehncrona S, Widner H, Lindvall O. 1999. Dopamine release from nigral transplants visualized in vivo in a Parkinson's patient. Nat Neurosci 2:1137-1140.
Prockop, D. J., Marrow stromal cells as stem cells for nonhematopoietic tissues. Science, 276 (5309) (1997) 71-74.
Rakhit R, Cunningham P, Furtos-Matei A, Dahan S, Qi X F, Crow J P, Cashman N R, Kondejewski L H, Chakrabartty A. 2002 Oxidation-induced misfolding and aggregation of superoxide dismutase and its implications for amyotrophic lateral sclerosis. J Biol Chem 277:47551-47556.
Redmond D E, Sladek J R, Roth R H, Collier T J, Elsworth J D, Deutsch A Y, Haber S. 1986. Fetal neuronal grafts in monkeys given methylphenyltetrahydropyridine. Lancet 1:1125-1127.
Rothstein J D, Martin L J, Kuncl R W. 1992. Decreased glutamate transport by the brain and spinal cord in amyotrophic lateral sclerosis. N Engl J Med 326:1464-1468.
Rowland L P, Shneider N A. 2001 Amyotrophic lateral sclerosis. N Engl J Med 344:1688-1700.
Sanchez-Ramos J R, Song S, Cardozo-Pelaez F, Hazzi C, Stedeford T, Willing A, Freeman T B, Saporta S, Janssen W, Patel N, Cooper D R, Sanberg P R. 2000. Adult bone marrow stromal cells differentiate into neural cells in vitro. Exp Neurol 164:247-256.
Sanchez-Ramos J R, Song S, Kamath S, Zigova T, Willing A, Cardozo-Pelaez, Stedeford T, Chopp M, Sanberg P R. 2001. Expression of neural markers in human umbilical cord blood. Exp Neurol 171:109-115.
Sanchez-Ramos J R. 2002. Neural cells derived from adult bone marrow and umbilical cord blood. J Neurosci Res 69:880-893.
Schwarz E J, Alexander G M, Prockop D J, Azizi A S. 1999. Multipotential marrow stromal cells transduced to produce L-DOPA: Engraftment in rat model of Parkinson disease. Hum Gene Ther 10:2539-2549.
Smith W C, Harland R M. 1992. Expression of cloning of noggin, a new dorsalizing factor localized to the Spemann organizer in Xenopus embryos. Cell 70:829-840.
Spencer D D, Robbins R J, Naftolin F, Marek K L, Vollmer T, Leranth C, Roth R H, Price L H, Gjedde A, Bunney B S, Sass K J, Elsworth J D, Kier E L, Makuch R, Hoffer B B, Redmond D E. 1992. Unilateral transplantation of human fetal mesencephalic tissue into the caudate nucleus of patients with Parkinson's disease. N Engl J Med 327:1541-1548.
Spivak B, Vered Y, Graff E, Blum I, Mester R, Weizman A. 1999. Low platelet-poor plasma concentrations of serotonin in patients with combat-related posttraumatic stress disorder. Biol Psychiatry 45:840-845.
Stathopulos P B, Rumfeldt J A, Scholz G A, Irani R A, Frey H E, Hallewell R A, Lepock J R, Meiering E M. 2003. Cu/Zn superoxide dismutase mutants associated with amyotrophic lateral sclerosis show enhanced formation of aggregates in vitro. Proc Natl Acad Sci USA May 28; [epub ahead of print].
Tham T N, Lazarini F, Franceschini I A, Lachapelle F, Amara A, Dubois-Dalcq M. 2001 Developmental pattern of expression of the alpha chemokine stromal cell-derived factor 1 in the rat central nervous system. Eur J Neurosci. March; 13(5):845-56
Tropepe V, Sibilia M, Ciruna B G, Rossant T, Wagner E F, van der Kooy D. 1999. Distinct neural stem cells proliferate in response to EGF and FGF in the developing mouse telencephalon. Dev Biol 208:166-188.
Urushitani M, Shimohama S. 2001. The role of nitric oxide in amyotrophic lateral sclerosis. Amyotroph Lateral Scler Other Motor Neuron Disord 2:71-81.
Vingerhoets F J, Schulzer M, Ruth T J, Holden J E, Snow B J. 1996. Reproducibility and discriminating ability of fluorine-18-6-fluoro-L-Dopa PET in Parkinson's disease. J Nucl Med 37:421-426.
Vingerhoets F J, Snow B J, Schulzer M, et al. 1994. Reproducibility of fluorine-18-6-fluorodopa positron emission tomography in normal human subjects. J Nucl Med 35:18-24.
Watanabe M, Dykes-Hoberg M, Culotta V C, Price D L, Wong P C, Rothstein J D. 2001. Histological evidence of protein aggregation in mutant SOD1 transgenic mice and in amyotrophic lateral sclerosis neural tissues. Neurobiol Dis 8:933-941.
Wenning G K, Odin P, Morrish P, Rehncrona S, Widner H, Brundin P, Rothwell J C, Brown R, Gustavii B, Hagell P, Jahanshahi M, Sawle G, Bjorklund A, Brooks D J, Marsden C D, Quinn N P, Lindvall O. 1997. Short- and long-term survival and function of unilateral intrastriatal dopaminergic grafts in Parkinson's disease. Ann Neurol. 42:95-107.
Widner H, Tetrud J, Rehncrona S, Snow B, Brundin P, Gustavii B, Bjorklund A, Lindvall O, Langston J W. 1992. Bilateral fetal mesencephalic grafting in two patients with parkinsonism induced by 1-methyl-4-phenyl-1,2,3,6-tetrahydropyridine (MPTP). N Engl J Med 327:1556-1563.
Woodbury D, Schwarz E J, Prockop D J, Black I B. 2000. Adult rat and human bone marrow stromal cells differentiate into neurons. J Neurosci Res 61:364-370.
Yadid G, Fitoussi N, Kinor R, Geffen R, Gispan I. 1999. Astrocyte line SVG-TH in rat model of Parkinson's disease. Prog Neurobiol 59:635-661.
Yamamoto A, Lucas J J, Hen R. 2000. Reversal of neuropathology and motor dysfunction in a conditional model of Huntington's disease. Cell 101:57-66.
Yoshimoto Y, Lin Q, Collier T J, Frim D M, Breakefield X O, Bohn M C. 1995. Astrocytes retrovirally transuded with BDNF elicit behavioral improvement in the rat model of Parkinson's disease. Brain Res 691:25-36.
Zhu Y, Yu T, Zhang X C, Nagasawa T, Wu J Y, Rao Y. 2002. Role of the chemokine SDF-1 as the meningeal attractant for embryonic cerebellar neurons. Nat Neurosci. August; 5(8):719-20
Zigova T, Song S, Willing A E, Hudson J E, Newman M B, Sorta S, Sanchez-Ramos J, Sanberg P R. 2002. Human umbilical cord blood cells express neural antigens after transplantation into the developing rat brain. Cell Transplant 11:265-274.

What is claimed is:

1. A method of producing cells for use in treating neurodegenerative disorders, comprising incubating bone marrow stromal cells in a differentiating medium comprising docosahexaenoic acid or arachidonic acid and at least one differentiating agent, thereby producing the cells for use in treating neurodegenerative disorders.

2. The method of claim 1 further comprising culturing said bone marrow stromal cells in a proliferation medium prior to said incubating said bone marrow stromal cells in a differentiating medium.

3. The method of claim 1, wherein said proliferation medium includes DMEM, SPN, L-glutamine, FCS, 2-β-mercaptoethanol, nonessential amino acids and EGF.

4. The method of claim 1, further comprising culturing said bone marrow stromal cells in an additional differentiating medium prior to said incubating said bone marrow stromal cells in a differentiating medium.

5. The method of claim 4, wherein said additional differentiating medium includes at least one of the agents selected from the group consisting of bFGF, EGF, vitamin E, FGF8, and shh.

6. The method of claim 4, wherein said additional differentiating medium further includes at least one polyunsaturated fatty acid.

7. The method of claim 6, wherein said at least one polyunsaturated fatty acid is docosahexaenoic acid or arachidonic acid.

8. The method of claim 4, wherein said additional differentiating medium further includes DMEM, SPN, L-glutamine, N2 supplement and FCS.

9. The method of claim 1, wherein said at least one differentiating agent is selected from the group consisting of BHA, ascorbic acid, BDNF, GDNF, NT-3, IL-1β, NTN, TGFβ3 and dbcAMP.

10. The method of claim 1, wherein said differentiating medium includes DMEM, SPN, L-glutamine, N2 supplement and retinoic acid.

11. The method of claim 1, wherein said differentiating medium comprises butylated hydroxyanisole (BHA), basic fibroblast growth factor (bFGF), epidermal growth factor (EGF), retinoic acid and a cAMP inducer selected from the group consisting of dibutyryl cyclic AMP (dbcAMP) and IBMX.

12. The method of claim 1, wherein said differentiating medium comprises butylated hydroxyanisole (BHA), basic fibroblast growth factor (bFGF), epidermal growth factor (EGF) and a cAMP inducer selected from the group consisting of dibutyryl cyclic AMP (dbcAMP) and IBMX; and at least one neuronal differentiating agent selected from the group consisting of brain-derived neurotrophic factor (BDNF) and glia derived neurotrophic factor (GDNF).

13. The method of claim 1, wherein said bone marrow stromal cells comprise human bone marrow stromal cells.

14. An isolated population of cells generated according to the method of claim 1.


SEQUENCE LISTING (66 sequences)

SEQ ID NO: 1 - DNA, 20 bp, Artificial sequence
  Single strand DNA oligonucleotide
ctagtggaccagagccttcg

SEQ ID NO: 2 - DNA, 20 bp, Artificial sequence
  Single strand DNA oligonucleotide
gccctcacacttgaccagtt

SEQ ID NO: 3 - DNA, 20 bp, Artificial sequence
  Single strand DNA oligonucleotide
aagaccatgagccgtaggaa

SEQ ID NO: 4 - DNA, 20 bp, Artificial sequence
  Single strand DNA oligonucleotide
gtacaggacaggcgggatgt

SEQ ID NO: 5 - DNA, 23 bp, Artificial sequence
  Single strand DNA oligonucleotide
gctcaccctgggtatcgacagcg

SEQ ID NO: 6 - DNA, 24 bp, Artificial sequence
  Single strand DNA oligonucleotide
atagaaccaggccactccgatggc

SEQ ID NO: 7 - DNA, 26 bp, Artificial sequence
  Single strand DNA oligonucleotide
ggattcagggcttatcactgactacc

SEQ ID NO: 8 - DNA, 28 bp, Artificial sequence
  Single strand DNA oligonucleotide
ttcattcactttgttggaaccctttagc

SEQ ID NO: 9 - DNA, 18 bp, Artificial sequence
  Single strand DNA oligonucleotide
ccatggagaaggctgggg

SEQ ID NO: 10 - DNA, 20 bp, Artificial sequence
  Single strand DNA oligonucleotide
caaagttgtcatggatgacc

SEQ ID NO: 11 - DNA, 18 bp, Artificial sequence
  Single strand DNA oligonucleotide
tgggaaaggcaaaagcag

SEQ ID NO: 12 - DNA, 20 bp, Artificial sequence
  Single strand DNA oligonucleotide
ctctctgcataaccaggaac

SEQ ID NO: 13 - DNA, 23 bp, Artificial sequence
  Single strand DNA oligonucleotide
tctcagatgtcctaaacgatgct

SEQ ID NO: 14 - DNA, 23 bp, Artificial sequence
  Single strand DNA oligonucleotide
caaggacttgcttgttaggaaga

SEQ ID NO: 15 - DNA, 20 bp, Artificial sequence
  Single strand DNA oligonucleotide
taatcccactgcccactacc

SEQ ID NO: 16 - DNA, 21 bp, Artificial sequence
  Single strand DNA oligonucleotide
tctaccccaacacacatcatc

SEQ ID NO: 17 - DNA, 18 bp, Artificial sequence
  Single strand DNA oligonucleotide
ccaagaccaaagcaaagg

SEQ ID NO: 18 - DNA, 22 bp, Artificial sequence
  Single strand DNA oligonucleotide
tttattggggggaaaaagtcag

SEQ ID NO: 19 - DNA, 19 bp, Artificial sequence
  Single strand DNA oligonucleotide
ccagaaactcaagcaccac

SEQ ID NO: 20 - DNA, 19 bp, Artificial sequence
  Single strand DNA oligonucleotide
ttttccactccagccatcc

SEQ ID NO: 21 - DNA, 20 bp, Artificial sequence
  Single strand DNA oligonucleotide
ctcaagggagtcatcaagga

SEQ ID NO: 22 - DNA, 20 bp, Artificial sequence
  Single strand DNA oligonucleotide
attggctgtgaacttggacc

SEQ ID NO: 23 - DNA, 25 bp, Artificial sequence
  Single strand DNA oligonucleotide
tgaacacagaacgctatgcgctcag

SEQ ID NO: 24 - DNA, 25 bp, Artificial sequence
  Single strand DNA oligonucleotide
cacctttatgtgagtggacacagag

SEQ ID NO: 25 - DNA, 20 bp, Artificial sequence
  Single strand DNA oligonucleotide
aagccactcagaccagaata

SEQ ID NO: 26 - DNA, 20 bp, Artificial sequence
  Single strand DNA oligonucleotide
gcagcgatttctatatccag

SEQ ID NO: 27 - DNA, 24 bp, Artificial sequence
  Single strand DNA oligonucleotide
aaaaggccggagaggtcgtttgcc

SEQ ID NO: 28 - DNA, 23 bp, Artificial sequence
  Single strand DNA oligonucleotide
tgggttccttgagcccgtgtctc

SEQ ID NO: 29 - DNA, 26 bp, Artificial sequence
  Single strand DNA oligonucleotide
agcagcagttctgaagagatagtgcc

SEQ ID NO: 30 - DNA, 18 bp, Artificial sequence
  Single strand DNA oligonucleotide
cgtcagcgtgtagctctc

SEQ ID NO: 31 - DNA, 28 bp, Artificial sequence
  Single strand DNA oligonucleotide
gcggatccaagccatgccttgtgttcag

SEQ ID NO: 32 - DNA, 32 bp, Artificial sequence
  Single strand DNA oligonucleotide
ggtctagattagaaaggtaaagtgtccaggaa

SEQ ID NO: 33 - DNA, 24 bp, Artificial sequence
  Single strand DNA oligonucleotide
ctaggccacagaattgaaagatct

SEQ ID NO: 34 - DNA, 25 bp, Artificial sequence
  Single strand DNA oligonucleotide
gtaggtggaaattctagcatcatcc

SEQ ID NO: 35 - DNA, 21 bp, Artificial sequence
  Single strand DNA oligonucleotide
catcagccctaatccatctga

SEQ ID NO: 36 - DNA, 21 bp, Artificial sequence
  Single strand DNA oligonucleotide
cgcgactaacaatcaaagtga

SEQ ID NO: 37 - DNA, 41 bp, Artificial sequence
  Single strand DNA oligonucleotide
tattacaattgtgtatgcagctggacctaggagagaagcag

SEQ ID NO: 38 - DNA, 41 bp, Artificial sequence
  Single strand DNA oligonucleotide
ttaatgaattccggtggtagtggcggtggcggtggcggtgg

SEQ ID NO: 39 - DNA, 31 bp, Artificial sequence
  Single strand DNA oligonucleotide
tattagctagcatgcccacccccgacgccac

SEQ ID NO: 40 - DNA, 31 bp, Artificial sequence
  Single strand DNA oligonucleotide
ttaatgctagcctagccaatggcactcagcg

SEQ ID NO: 41 - DNA, 19 bp, Artificial sequence
  Single strand DNA oligonucleotide
agacccgactcagtttcac

SEQ ID NO: 42 - DNA, 20 bp, Artificial sequence
  Single strand DNA oligonucleotide
accttcacctccttcttctt

SEQ ID NO: 43 - DNA, 20 bp, Artificial sequence
  Single strand DNA oligonucleotide
taacctcactgtggaggaag

SEQ ID NO: 44 - DNA, 20 bp, Artificial sequence
  Single strand DNA oligonucleotide
cccgttatagaaccactgaa

SEQ ID NO: 45 - DNA, 20 bp, Artificial sequence
  Single strand DNA oligonucleotide
acaaactcctggtgcaaacc

SEQ ID NO: 46 - DNA, 20 bp, Artificial sequence
  Single strand DNA oligonucleotide
ctttgtcgtggacccattct

SEQ ID NO: 47 - DNA, 20 bp, Artificial sequence
  Single strand DNA oligonucleotide
tgtggagacccaagacatca

SEQ ID NO: 48 - DNA, 20 bp, Artificial sequence
  Single strand DNA oligonucleotide
ccgagacgatgaaggagaag

SEQ ID NO: 49 - DNA, 20 bp, Artificial sequence
  Single strand DNA oligonucleotide
gggaggctacttcctcatcc

SEQ ID NO: 50 - DNA, 20 bp, Artificial sequence
  Single strand DNA oligonucleotide
ggcagctgaaggtaatgagc

SEQ ID NO: 51 - DNA, 20 bp, Artificial sequence
  Single strand DNA oligonucleotide
atgagtttgtggggaagacg

SEQ ID NO: 52 - DNA, 20 bp, Artificial sequence
  Single strand DNA oligonucleotide
tgtttgcaaagcagatggag

SEQ ID NO: 53 - DNA, 19 bp, Artificial sequence
  Single strand DNA oligonucleotide
tcaaaactgactcgcagca

SEQ ID NO: 54 - DNA, 20 bp, Artificial sequence
  Single strand DNA oligonucleotide
actccgccttgagtctctgc

SEQ ID NO: 55 - DNA, 20 bp, Artificial sequence
  Single strand DNA oligonucleotide
ggcgaaccctgactatcaaa

SEQ ID NO: 56 - DNA, 20 bp, Artificial sequence
  Single strand DNA oligonucleotide
accccattgcaaaagatgag

SEQ ID NO: 57 - DNA, 20 bp, Artificial sequence
  Single strand DNA oligonucleotide
aggagctagaggcgaccttc

SEQ ID NO: 58 - DNA, 20 bp, Artificial sequence
  Single strand DNA oligonucleotide
aagctgcctttgcatagctc

SEQ ID NO: 59 - DNA, 20 bp, Artificial sequence
  Single strand DNA oligonucleotide
tcgacaacaaatggaggaca

SEQ ID NO: 60 - DNA, 20 bp, Artificial sequence
  Single strand DNA oligonucleotide
cagagggggattgaagtgaa

SEQ ID NO: 61 - DNA, 23 bp, Artificial sequence
  Single strand DNA oligonucleotide
tctcagatgtcctaaacgatgct

SEQ ID NO: 62 - DNA, 23 bp, Artificial sequence
  Single strand DNA oligonucleotide
caaggacttgcttgttaggaaga

SEQ ID NO: 63 - DNA, 20 bp, Artificial sequence
  Single strand DNA oligonucleotide
tcgacaacaaatggaggaca

SEQ ID NO: 64 - DNA, 20 bp, Artificial sequence
  Single strand DNA oligonucleotide
cagagggggattgaagtgaa

SEQ ID NO: 65 - DNA, 20 bp, Artificial sequence
  Single strand DNA oligonucleotide
tgttagctgatgccgacttg

SEQ ID NO: 66 - DNA, 20 bp, Artificial sequence
  Single strand DNA oligonucleotide
ttcttagcccgctcaacact